(19) United States
(12) Patent Application Publication
(10) Pub. No.: US 2018/0153795 A1
(43) Pub. Date: Jun. 7, 2018

(54) LIQUID BUPRENORPHINE FORMULATIONS

(71) Applicant: Insys Development Company, Inc., Chandler, AZ (US)

(72) Inventors: Kiran P. Amancha, Chandler, AZ (US); Chandeshwari S. Chilampalli, Phoenix, AZ (US); Venkat R. Goskonda, Phoenix, AZ (US)

(21) Appl. No.: 15/887,655

(22) Filed: Feb. 2, 2018

Related U.S. Application Data
(63) Continuation-in-part of application No. 15/336,547, filed on Oct. 27, 2016, now Pat. No. 9,918,981.
Application No. 15/336,547 is a continuation-in-part of application No. 14/923,630, filed on Oct. 27, 2015, now Pat. No. 9,839,611.
Application No. 14/923,630 is a continuation-in-part of application No. 14/469,063, filed on Aug. 26, 2014, now Pat. No. 9,216,175.
(60) Provisional application No. 61/875,837, filed on Sep. 10, 2013.

(51) Int. Cl. A61K 9/00; A61K 31/485; A61K 47/10; A61K 45/06; A61P 25/36

(52) U.S. Cl. CPC A61K 9/006; A61K 31/485; A61P 25/36; A61K 45/06; A61K 47/10

(57) ABSTRACT

The invention provides liquid formulations containing buprenorphine, a pharmaceutically acceptable salt thereof, or a derivative thereof. The invention further provides liquid formulations containing buprenorphine and naloxone, pharmaceutically acceptable salts thereof or derivatives thereof. The invention further provides a method of treating pain or opioid dependence by administering liquid formulations containing buprenorphine or a combination of buprenorphine and naloxone, a pharmaceutically acceptable salt thereof, or a derivative thereof to a patient in need thereof.

### FIELD OF THE INVENTION

The invention is directed to liquid formulations containing buprenorphine, a pharmaceutically acceptable salt thereof, or a derivative thereof. The invention is further directed to liquid formulations containing buprenorphine and naloxone, pharmaceutically acceptable salts thereof or derivatives thereof. The invention is further directed to a method of treating pain or opioid dependence by administering liquid formulations containing buprenorphine or buprenorphine and naloxone, pharmaceutically acceptable salts thereof, or derivatives thereof to a patient in need thereof.

### BACKGROUND OF THE INVENTION

Buprenorphine is a semi-synthetic opioid and a partial μ-opioid receptor agonist and has the following structure:

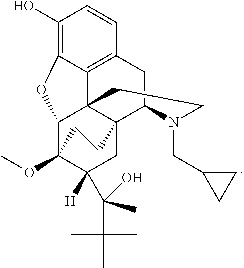 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

Activation of the μ-opioid receptor leads to antinociception and is the pathway by which opioids such as morphine and fentanyl reduce acute and chronic pain. Buprenorphine has advantages over other opioids such as morphine and fentanyl in that it is only a partial instead of a full agonist of the opioid receptor-like receptor 1 (“ORL1”). Activation of ORL1 has been reported to weaken the analgesic effect induced by the activation of the μ-opioid receptor. Additionally, buprenorphine is an antagonist of δ- and κ-opioid receptors, whose activation has anti-analgesic and psychotomimetic effects, respectively. Buprenorphine is also useful in the management of opioid dependence. The slow binding of buprenorphine to the μ-opioid receptor along with its strong affinity allows for pain management at relatively low blood concentrations and the slow disassociation of buprenorphine from the μ-opioid receptor results in a lack of withdrawal symptoms.

Buprenorphine is currently available in transdermal patches, intravenous injection, tablet and film strip formulations. Commercially available buprenorphine formulations include Butrans® (Butrans is a registered trademark of Purdue Pharma L.P.), a 7 day transdermal patch that releases buprenorphine at 5, 10 or 20 mcg/hr, and Temgesic, a 0.2 mg sublingual tablet, are used for the treatment of chronic pain. Buprenex® (Buprenex is a registered trademark of Reckitt Benckiser Healthcare (UK) Limited) is a 0.3 mg/mL injectable solution used for the treatment of acute pain. Subutex® (Subutex is a registered trademark of Reckitt Benckiser Healthcare (UK) Limited) and Suboxone® (Suboxone is a registered trademark of Reckitt Benckiser Healthcare (UK) Limited) are tablets used in the treatment of opioid dependence. Subutex® is available in 2 mg and 8 mg sublingual doses of buprenorphine. Suboxone® contains both buprenorphine and naloxone in a 4:1 ratio. Suboxone® is available in tablet form in 2 mg and 8 mg doses. Suboxone® is also available in a sublingual film strip formulation that dissolves faster and is not lost by accidental swallowing.

Naloxone has the following structure and is synthesized from thebaine:

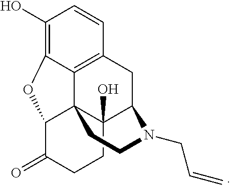

Naloxone is most commonly used to treat patients suffering from opioid dependence or overdose because it is a competitive μ-opioid antagonist that blocks the effects of opioids.

While there are various formulations currently available, there exists a need in the art for a liquid (i.e., sublingual or intranasal) spray formulation containing buprenorphine or buprenorphine and naloxone, pharmaceutically acceptable salts thereof, or derivatives thereof. Such a formulation should be safe, be easy to administer, have a high bioavailability, and be storage stable.

### SUMMARY OF THE INVENTION

In one embodiment, the present invention is directed to a liquid formulation comprising an effective amount of buprenorphine, a pharmaceutically acceptable salt thereof, or a derivative thereof, water as a solvent, and a mixture of an alcohol and a glycol as a cosolvent.

In one embodiment, the present invention is directed to a liquid formulation comprising:
from about 0.05% to about 10% w/w, preferably from about 0.07% to about 1.3% w/w of buprenorphine, a pharmaceutically acceptable salt thereof, or a derivative thereof;
optionally, naloxone, a pharmaceutically acceptable salt thereof, or a derivative thereof, preferably at a concentration from about 0.005% to about 3% w/w;
optionally, an antioxidant, preferably selected from the group consisting of butylated hydroxyanisole (BHA), butylated hydroxytoluene (BHT), methionine, sodium ascorbate, sodium thiosulfate, thioglycerol, cysteine hydrochloride monohydrate and a mixture thereof;
water as a solvent; and
a mixture of an alcohol and a glycol as a cosolvent.

In a preferred embodiment the liquid formulation is in the form of a liquid spray.

In one embodiment, the present invention is directed to a liquid formulation comprising:
From about 0.07% to about 1.3% w/w of buprenorphine, a pharmaceutically acceptable salt thereof, or a derivative thereof;
water as a solvent in an amount from about 38% to about 40% w/w;
a cosolvent consisting of a mixture of ethanol in an amount of about 55% w/w and propylene glycol in an amount of about 5% w/w;
an antioxidant in an amount from about 0.0001% to about 0.5% w/w, preferably a mixture of butylated hydroxyanisole (BHA) in an amount of about 0.01% w/w and butylated hydroxytoluene (BHT) in an amount of about 0.005% w/w;
optionally, from about 0.005% to about 0.5% w/w menthol, preferably at about 0.05% w/w.

In another embodiment, the liquid formulations of the present invention are optionally, in a sublingual spray form, and are capable of producing:
a droplet size distribution wherein greater than 98% of the composition particles are greater than 10 microns in diameter during administration;
a droplet size distribution wherein the mean Dv(10) is from about 10 to about 40 microns during administration, the mean Dv(50) is from about 30 to about 80 microns during administration, and the mean Dv(90) is from about 80 to about 200 microns during administration;
a spray plume that has an ovality ratio of from about 1.1 to 2.4;
a spray plume width that is from about 25 to about 45 millimeters during administration and a spray plume angle that is from about 30 to about 55 degrees during administration;
a D(4,3) of 50 to 95 microns;
a droplet size distribution wherein the Tmax of buprenorphine is from about 0.5 to about 2.0 hours following administration;
a droplet size distribution wherein the AUCinf of buprenorphine (h*ng/mL) is from 0.6387±0.1844 to 11.36±3.153;
a droplet size distribution wherein the Cmax (ng/mL) of buprenorphine is from 2470±850 to 5670±1590 and the Cmax (ng/mL) of naloxone is from 4.26±2.52 to 12.0±5.38;
a droplet size distribution wherein the Tmax of buprenorphine is from 1.63±0.5 to 1.68±0.73 hours and the Tmax of naloxone is from about 1.17 to about 1.40 hours following administration; and/or
a droplet size distribution wherein the AUCinf (h*ng/mL) of buprenorphine is from 19320±6190 to 48970±13810 and the AUCinf (h*ng/mL) of naloxone is from 11.87±3.903 to 36.22±10.45.

In one embodiment, the present invention is directed to a liquid formulation comprising:
an effective amount of buprenorphine, a pharmaceutically acceptable salt thereof, or a derivative thereof;
water as a solvent;
a cosolvent selected from the group consisting of an alcohol and a glycol or a mixture thereof; and
an antioxidant.

In one embodiment, the present invention is directed to a liquid formulation comprising:
an effective amount of buprenorphine, a pharmaceutically acceptable salt thereof, or a derivative thereof;
water as a solvent;
a cosolvent selected from the group consisting of an alcohol and a glycol or a mixture thereof; and
an antioxidant.

In one embodiment, the present invention is directed to a liquid formulation comprising:
an effective amount of buprenorphine, a pharmaceutically acceptable salt thereof, or a derivative thereof;
water as a solvent;
a mixture of an alcohol and a glycol as a cosolvent; and
an antioxidant selected from the group consisting of butylated hydroxyanisole (“BHA”), butylated hydroxytoluene (“BHT”), methionine, sodium ascorbate, sodium thiosulfate, thioglycerol, cysteine hydrochloride monohydrate and a mixture thereof.

In one embodiment, the present invention is directed to a liquid formulation comprising:
an effective amount of buprenorphine, a pharmaceutically acceptable salt thereof, or a derivative thereof;
water as a solvent;
a mixture of ethanol and propylene glycol as a cosolvent; and
an antioxidant selected from the group consisting of BHA, BHT, methionine, sodium ascorbate, sodium thiosulfate and thioglycerol, cysteine hydrochloride monohydrate or a mixture thereof.

In one embodiment, the present invention is directed to a liquid formulation comprising:
an effective amount of buprenorphine, a pharmaceutically acceptable salt thereof, or a derivative thereof;
water as a cosolvent;
a cosolvent selected from the group consisting of ethanol, propylene glycol, and a mixture thereof;
an antioxidant selected from the group consisting of BHA, BHT, methionine, sodium ascorbate, sodium thiosulfate, thioglycerol, cysteine hydrochloride monohydrate and a mixture thereof; and
a permeation enhancer.

In one embodiment, the present invention is directed to a liquid formulation comprising:
an effective amount of buprenorphine, a pharmaceutically acceptable salt thereof, or a derivative thereof;
water as a solvent;
a cosolvent selected from the group consisting of ethanol, propylene glycol, and a mixture thereof;
an antioxidant selected from the group consisting of BHA, BHT, methionine, sodium ascorbate, sodium thiosulfate, thioglycerol, cysteine hydrochloride monohydrate and a mixture thereof; and
menthol as a permeation enhancer.

In one embodiment, the present invention is directed to a liquid formulation comprising:
an effective amount of buprenorphine, a pharmaceutically acceptable salt thereof, or a derivative thereof;
water as a solvent;
a cosolvent selected from the group consisting of ethanol, propylene glycol, and a mixture thereof;
an antioxidant selected from the group consisting of BHA, BHT, methionine, sodium ascorbate, sodium thiosulfate, thioglycerol, cysteine hydrochloride monohydrate and a mixture thereof; and
a pH adjustor.

In one embodiment, the present invention is directed to a liquid formulation comprising:
an effective amount of buprenorphine, a pharmaceutically acceptable salt thereof, or a derivative thereof;
water as a solvent;
a cosolvent selected from the group consisting of ethanol, propylene glycol, and a mixture thereof;
an antioxidant selected from the group consisting of BHA, BHT, methionine, sodium ascorbate, sodium thiosulfate, thioglycerol, cysteine hydrochloride monohydrate and a mixture thereof; and
citric acid as a pH adjustor selected from the group consisting of citric acid, sodium hydroxide and a mixture thereof.

In one embodiment, the present invention is directed to a liquid formulation comprising:
an effective amount of buprenorphine, a pharmaceutically acceptable salt thereof, or a derivative thereof;
water as a solvent;
a solubilizer selected from the group consisting of cyclodextrins such as hydroxpropyl beta-cyclodextrin (“HPβCD”), sulfobutylether cyclodextrin, and a mixture thereof; and
an antioxidant selected from the group consisting of BHA, BHT, methionine, sodium ascorbate, sodium thiosulfate, thioglycerol, cysteine hydrochloride monohydrate and a mixture thereof.

When the application describes the amounts of buprenorphine and naloxone, all the amounts refer to buprenorphine base and naloxone base, respectively, unless otherwise indicated.

In one embodiment, the present invention is directed to a sublingual spray formulation comprising:
an amount of buprenorphine from about 0.01% to about 10% w/w;
an amount of water from about 10% to about 95% w/w;
an amount of ethanol as a cosolvent from about 10% to about 80% w/w;
a glycol in an amount from about 0.5% to about 50% w/w; and
an amount of antioxidant from about 0.0001% to about 0.5% w/w; and
optionally, menthol in an amount of about 0.005% w/w to about 0.5% w/w as a permeation enhancer.

In one embodiment, the present invention is directed to a sublingual spray formulation comprising:
an amount of buprenorphine from about 0.06% to about 1.5% w/w;
an amount of water from about 38% to about 40% w/w;
a cosolvent consisting of a mixture of ethanol in an amount of 55% w/w and propylene glycol in an amount of about 5% w/w;
an antioxidant consisting of a mixture of butylated hydroxyanisole (BHA) in an amount of about 0.01% w/w and butylated hydroxytoluene (BHT) in an amount of about 0.005% w/w; and
menthol in an amount of about 0.05% w/w.

In one embodiment, the present invention is directed to a sublingual spray formulation comprising:
buprenorphine, a pharmaceutically acceptable salt thereof or a derivative thereof in an amount from about 0.05% to about 5% w/w;
water as a solvent in an amount from about 20% to about 60% w/w;
a cosolvent consisting of a mixture of an alcohol from about 30% w/w to about 60% w/w and a glycol in an amount from about 1% to about 10% w/w;
an antioxidant in an amount from about 0.001% to about 0.1% w/w; and
menthol from about 0.01% w/w to about 0.1% w/w;
wherein the % w/w is of the total formulation.

In one embodiment, the present invention is directed to a sublingual spray formulation comprising:
buprenorphine, a pharmaceutically acceptable salt thereof or a derivative thereof in an amount from about 0.06% to about 1.5% w/w;
water as a solvent in an amount of from about 38% to about 40% w/w;
a cosolvent consisting of a mixture of ethanol in an amount of 55% w/w and propylene glycol in an amount of about 5% w/w;
the antioxidant consisting of a mixture of butylated hydroxyanisole (BHA) in an amount of about 0.01% w/w and butylated hydroxytoluene (BHT) in an amount of about 0.005% w/w; and
menthol at an amount of about 0.05% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.05% to about 15% w/w;
naloxone, a pharmaceutically acceptable salt thereof or a derivative thereof at an amount from about 0.005% to about 5% w/w;
water as a solvent in an amount from about 10% w/w to about 95% w/w;
a cosolvent consisting of a mixture of an alcohol in an amount from about 10% to about 80% w/w and a glycol in an amount from about 0.5% w/w to about 50% w/w;
an antioxidant in an amount from about 0.001% to about 0.2% w/w; and
a chelating agent in an amount from about 0.001% to about 0.1% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.05% to about 10% w/w;
naloxone, a pharmaceutically acceptable salt thereof or a derivative thereof at an amount from about 0.1% to about 3% w/w;
water as a solvent in an amount from about 20% w/w to about 45% w/w;
a cosolvent consisting of a mixture of ethanol in an amount of 50% w/w to about 60% w/w and propylene glycol in an amount of about 4% w/w to 6% w/w;
an antioxidant selected from a group consisting of butylated hydroxyanisole, butylated hydroxytoluene, methionine, sodium ascorbate, sodium thiosulfate, thioglycerol, cysteine hydrochloride monohydrate, and a mixture thereof at an amount of about 0.01% to about 0.1 w/w;
disodium edetate as a chelating agent at an amount of about 0.001% to about 0.01% w/w; and
menthol at an amount of about 0.005% to 0.5% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.6% to about 10% w/w;
naloxone, a pharmaceutically acceptable salt thereof or a derivative thereof at an amount from about 0.1% to about 3.0% w/w;
menthol at an amount of about 0.05% w/w;
disodium edetate at an amount of about 0.005% w/w;
sodium ascorbate in an amount of about 0.02%;
ethanol in an amount of about 55%;
propylene glycol in an amount from about 5% w/w;
water in an amount from about 25% w/w to 40% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.05% to about 9.5% w/w;
naloxone, a pharmaceutically acceptable salt thereof or a derivative thereof at an amount from about 0.2% to about 2.7% w/w;
water as a solvent in an amount from about 27.4% w/w to 39.7% w/w;
a cosolvent consisting of a mixture of ethanol in an amount from about 55% w/w and propylene glycol in an amount from about 5% w/w; and
an antioxidant selected from a group consisting of butylated hydroxyanisole, butylated hydroxytoluene, methionine, sodium ascorbate, sodium thiosulfate, thioglycerol, cysteine hydrochloride monohydrate and a mixture thereof in an amount from about 0.001% to about 0.2% w/w.

In one embodiment, the present invention is directed to a sublingual spray formulation comprising:
buprenorphine, a pharmaceutically acceptable salt thereof or a derivative thereof at an amount from about 0.05% to about 9.5% w/w;
naloxone, a pharmaceutically acceptable salt thereof or a derivative thereof at an amount from about 0.005% to about 2.7% w/w;
water as a solvent in an amount from about 27.4% w/w to 39.7% w/w;
a cosolvent consisting of a mixture of ethanol in an amount of about 55% w/w and propylene glycol in an amount of about 5% w/w; and
an antioxidant selected from a group consisting of butylated hydroxyanisole, butylated hydroxytoluene, methionine, sodium ascorbate, sodium thiosulfate, thioglycerol, cysteine hydrochloride monohydrate, and a mixture thereof in an amount from about 0.001% to about 0.2% w/w.

In one embodiment, the present invention is directed to a sublingual spray formulation comprising:
buprenorphine, a pharmaceutically acceptable salt thereof or a derivative thereof at an amount from about 0.05% to about 9.5% w/w;
naloxone, a pharmaceutically acceptable salt thereof or a derivative thereof at an amount from about 0.005% to about 3% w/w;
water as a solvent in an amount from about 27.4% w/w to 39.7% w/w;
a cosolvent consisting of a mixture of ethanol in an amount of about 55% w/w and propylene glycol in an amount of about 5% w/w;
an antioxidant selected from a group consisting of butylated hydroxyanisole, butylated hydroxytoluene, methionine, sodium ascorbate, sodium thiosulfate, thioglycerol, cysteine hydrochloride monohydrate, and a mixture thereof; and
ethylenediaminetetraacetic acid disodium (disodium edetate) as a chelating agent in an amount of about 0.005% w/w or citric acid as a pH adjustor in an amount from about 0.0025 to 10% w/w.

In certain embodiments, the liquid formulations are the liquid spray formulations.

In certain embodiments, the liquid formulations of the present invention contain naloxone in an amount that discourages improper administration of the formulations. When the naloxone containing formulations are properly administered, the naloxone is delivered at a rate that is below that which would be therapeutic. In this context, “therapeutic” refers to an amount of naloxone that would block the effects of the buprenorphine that is concurrently administered in the sublingual spray formulation. If the formulations are improperly used, however, the naloxone in the formulation could be sufficient to block the effects of buprenorphine.

In certain embodiments, the present invention is directed to methods for treating pain comprising administering a liquid formulation of the present invention to a patient.

In certain embodiments, the present invention is directed to methods for treating opioid dependence comprising administering a liquid formulation of the present invention to a patient.

In an embodiment, the present invention is directed to sublingual spray formulations wherein the Cₘₐₓ (ng/mL) of buprenorphine is from 0.125±0.0203 to 1.57±0.453. In one preferred embodiment, the Cₘₐₓ (ng/mL) of buprenorphine is 0.76 following sublingual administration. In another preferred embodiment, the Cₘₐₓ (ng/mL) of buprenorphine is 1.38 following sublingual administration.

In yet another embodiment, the present invention is directed to sublingual spray formulations wherein the Tₘₐₓ of buprenorphine is from about 0.5 to about 2.0 hours. In a preferred embodiment, the Tₘₐₓ of buprenorphine is about 1.75 hours following sublingual administration.

In yet another embodiment, the present invention is directed to sublingual spray formulations wherein the Cₘₐₓ (ng/mL) of buprenorphine is from about 1.2 to about 1.5. In a preferred embodiment, the Cₘₐₓ (ng/mL) of buprenorphine is about 1.38 following sublingual administration.

In a further embodiment, the present invention is directed to sublingual spray formulations wherein the Tₘₐₓ of buprenorphine is from about 1.2 to about 1.7 hours. In a preferred embodiment, the Tₘₐₓ of buprenorphine is about 1.5 hours following sublingual administration.

In a further embodiment, the present invention is directed to sublingual spray formulations wherein the AUC_{0-T} (ng·h/mL) of buprenorphine is from about 2 to about 6 for 0.5 mg dose, and from about 7 to about 11 for 1 mg dose.

In a further embodiment, the present invention is directed to sublingual spray formulations wherein the AUC_{0-∞} (ng·h/mL) of buprenorphine is from about 2 to about 6 for 0.5 mg dose, and from about 7 to about 11 for 1 mg dose.

In a further embodiment, the present invention is directed to sublingual spray formulations wherein the AUCinf (h*ng/mL) of buprenorphine is from 0.6387±0.1844 to 11.36±3.153.

In a further embodiment, the present invention is directed to sublingual spray formulations wherein the Cmax (ng/mL) of naloxone is from 4.26±2.52 to 12.0±5.38

In a further embodiment, the present invention is directed to sublingual spray formulations wherein the Tₘₐₓ of naloxone is from about 1.17 to about 1.40 hours following administration.

In a further embodiment, the present invention is directed to sublingual spray formulations wherein the AUCinf (h*ng/mL) of naloxone is from 11.87±3.903 to 36.22±10.45.

In another embodiment, the present invention is directed to sublingual spray formulations wherein greater than 98% of the formulation particles are greater than 10 microns in diameter during administration.

In another embodiment, the present invention is directed to sublingual spray formulations wherein the mean Dv(10) is from about 10 to about 40 microns during administration.

In another embodiment, the present invention is directed to sublingual spray formulations wherein the mean Dv(50) is from about 30 to about 80 microns during administration.

In another embodiment, the present invention is directed to sublingual spray formulations wherein the mean Dv(90) is from about 80 to about 200 microns during administration.

In a further embodiment, the present invention is directed to sublingual spray formulations that when administered provide a spray plume ovality ratio of from about 1.1 to 2.4.

In yet another embodiment, the invention is directed to sublingual formulations that when administered provide a plume width of from about 25 to about 45 millimeters.

In a further embodiment, the invention is directed to sublingual formulations that when administered provide a plume angle of from about 30 to about 55 degrees.

In yet another embodiment, the invention is directed to sublingual formulations that when administered provide a D(4,3) of 55 to 95 microns.

In an additional embodiment, the invention is directed to sublingual formulations that when administered provide a spray span ((Dv90−Dv10)/Dv50) of from about 1.2 to about 3.3.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a flow chart describing the disposition of the study of the effect of buprenorphine sublingual spray to treat bunionectomy-related pain.

FIG. 2 depicts a chart of a chart of Numeric Rating Scale (NRS) Summed Pain Intensity Difference (SPID) at 4, 8, 24 and 48 hours.

FIG. 3 depicts a chart of time of onset of analgesia for placebo, 0.5 mg tid, 0.25 mg tid and 0.125 tid doses.

### DETAILED DESCRIPTION OF THE INVENTION

The present invention is directed to a liquid formulation comprising an effective amount of buprenorphine or buprenorphine and naloxone, pharmaceutically acceptable salts thereof, or derivatives thereof. The present invention further relates to a method of treating pain or opioid dependence by administering an effective amount of a liquid formulation of the present invention to a patient in need thereof.

The present invention is further directed to a liquid formulation comprising an effective amount of buprenorphine or buprenorphine and naloxone, pharmaceutically acceptable salts thereof, or derivatives thereof, a solvent, a cosolvent and an antioxidant.

Applicants developed new liquid buprenorphine and buprenorphine/naloxone formulations that unexpectedly are storage stable, safe and effective. Specifically, Applicants were surprised that the formulations were stable at high temperatures (40 degrees Celsius) for an extended period of time (see Examples 1 and 2 below). Further, Applicants unexpectedly found that the formulations provided a quick onset of action and bioavailability (as demonstrated by pharmacokinetic studies, see Example 3 below). The formulations upon administration exhibit excellent droplet size distribution, as well.

As used herein the term “patient” refers but is not limited to a person that is being treated for pain, opioid dependence or another affliction or disease that can be treated with buprenorphine.

As used herein the term “pharmaceutically acceptable” refers to ingredients that are not biologically or otherwise undesirable in a sublingual dosage form.

As used herein the term “effective amount” refers to the amount necessary to treat a patient in need thereof.

As used herein the term “liquid” refers to a sublingual, intranasal or otherwise administered through a mouth or a nose formulation.

As used herein the term “sublingual” refers to administration of a substance via the mouth in such a way that the substance is rapidly absorbed via the blood vessels under the tongue.

As used herein the term “intranasal” refers to administration of the composition to any portion of the nasal epithelium.

Pharmaceutically acceptable salts that can be used in accordance with the current invention include but are not limited to hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

In preferred embodiments the pharmaceutically acceptable salt is hydrochloride.

Derivatives of buprenorphine that can be used in accordance with the current invention include but are not limited norbuprenorphine, thenorphine, demethoxybuprenorphine and esters and diastereomers of buprenorphine.

The solvent used with the present invention is United States Pharmacopeia (“USP”) purified water.

Cosolvents that can be used in accordance with the current invention are alcohols, and glycols or a mixture thereof.

Alcohols that can be used in accordance with the current invention include but are not limited to methanol, ethanol, propyl alcohol, and butyl alcohol.

Glycols that can be used in accordance with the current invention include but are not limited to propylene glycol, butylene glycol and polyethylene glycols such as PEG 200 and PEG 400 and the like.

In preferred embodiments the cosolvent is ethanol or propylene glycol or a mixture thereof.

In more preferred embodiments the amount of cosolvent included in the formulation is from about 5% to about 90% w/w.

In other more preferred embodiments the amount of cosolvent included in the formulation is from about 2 to about 10% propylene glycol. In a most preferred embodiment the amount of cosolvent is about 5% w/w propylene glycol.

In other more preferred embodiments the amount of cosolvent included in the formulation is about 40% w/w to about 60% w/w ethanol. In a most preferred embodiment the amount of cosolvent is about 55% w/w ethanol.

In other more preferred embodiments the cosolvent is a mixture of propylene glycol at about 5% w/w and ethanol at about 55% w/w.

Solubilizers that can be used in accordance with the current invention are hydroxpropyl beta-cyclodextrin (“HPβCD”) and sulfobutylether cyclodextrin or a mixture thereof.

In preferred embodiments the solubilizer is HPβCD.

In more preferred embodiments the amount of HPβCD is from about 10% w/w to 40% w/w. In a most preferred embodiment the amount of HPβCD is about 30% w/w.

Antioxidants that can be used in accordance with the current invention include but are not limited to butylated hydroxyanisole (“BHA”), butylated hydroxytoluene (“BHT”), methionine, sodium ascorbate, sodium thiosulfate and thioglycerol, cysteine hydrochloride monohydrate or a mixture thereof.

In preferred embodiments the amount of antioxidant included in the formulation is from about 0.001% to about 0.05% w/w.

In more preferred embodiments the amount of antioxidant is about 0.01% w/w of BHA.

In other more preferred embodiments the antioxidant is a mixture of about 0.01% w/w of BHA and about 0.005% w/w of BHT.

In other more preferred embodiments the antioxidant is about 0.01% w/w of sodium thiosulfate.

In other more preferred embodiments the antioxidant is about 0.02% w/w of sodium ascorbate.

Permeation enhancers that can be used in accordance with the current invention include but are not limited to menthol, Tween® 80 (Tween is a registered trademark of Uniqema Americas, LLC), sodium lauryl sulfate, glyceryl oleate, oleic acid, cetylpyridium chloride, and sodium desoxy cholate.

In preferred embodiments the amount of permeation enhancer is from about 0.001% to about 0.1% w/w.

In more preferred embodiments the amount of permeation enhancer is about 0.05% w/w of menthol.

Chelating agents that can be used in accordance with the present invention include but are not limited to ethylenediaminetetraacetic acid disodium (“disodium edetate” or edetate disodium dihydrate”).

In preferred embodiments the amount of disodium edetate is about 0.005% to about 0.01% w/w.

Formulations of the present invention may have a pH range from about 3.0 to about 7.0, preferably from about 3.5 to about 5.5 and more preferably from about 3.8 to about 5.1. pH adjustors that can be used in accordance with the present invention include but are not limited to citric acid, sodium hydroxide and a mixture thereof. In preferred embodiments the amount of citric acid is from about 2% to about 20% w/w. In more preferred embodiments the amount of citric acid is about 15%. In other more preferred embodiments the amount of citric acid is about 10%.

As used herein, all numerical values relating to amounts, weights, and the like, that are defined as “about” each particular value is plus or minus 10%. For example, the phrase “about 10% w/w” is to be understood as “9% to 11% w/w.” Therefore, amounts within 10% of the claimed value are encompassed by the scope of the claims.

As used herein “% w/w” refers to the percent weight of the total formulation.

### Representative Embodiments

In one embodiment, the present invention is directed to a sublingual spray formulation comprising:
an amount of buprenorphine from about 0.01% to about 10% w/w;
an amount of water from about 10% to about 95% w/w;
an amount of cosolvent from about 10% to about 80% w/w;
a glycol in an amount from about 0.5% to about 50% w/w; and
an amount of antioxidant from about 0.0001% to about 0.5% w/w; and
optionally, menthol in an amount of about 0.005% w/w to about 0.5% w/w as a permeation enhancer.

In one embodiment, the present invention is directed to a sublingual spray formulation comprising:
an amount of buprenorphine from about 0.06% to about 1.5% w/w;
an amount of water from about 38% to about 40% w/w;
a cosolvent consisting of a mixture of ethanol in an amount of 55% w/w and propylene glycol in an amount of about 5% w/w;
an antioxidant consisting of a mixture of butylated hydroxyanisole (BHA) in an amount of about 0.01% w/w and butylated hydroxytoluene (BHT) in an amount of about 0.005% w/w; and
menthol in an amount of about 0.05% w/w.

In one embodiment, the present invention is directed to a sublingual spray formulation comprising:
buprenorphine, a pharmaceutically acceptable salt thereof or a derivative thereof in an amount from about 0.05% to about 5% w/w;
water as a solvent in an amount from about 20% to about 60% w/w;
a cosolvent consisting of a mixture of an alcohol from about 30% w/w to about 60% w/w and a glycol in an amount from about 1% to about 10% w/w;
an antioxidant in an amount from about 0.001% to about 0.1% w/w; and
menthol from about 0.01% w/w to about 0.1% w/w;
wherein the % w/w is of the total formulation.

In one embodiment, the present invention is directed to a sublingual spray formulation comprising:
buprenorphine, a pharmaceutically acceptable salt thereof or a derivative thereof in an amount from about 0.06% to about 1.5% w/w;
water as a solvent in an amount of from about 38% to about 40% w/w;
a cosolvent consisting of a mixture of ethanol in an amount of 55% w/w and propylene glycol in an amount of about 5% w/w;
the antioxidant consisting of a mixture of butylated hydroxyanisole (BHA) in an amount of about 0.01% w/w and butylated hydroxytoluene (BHT) in an amount of about 0.005% w/w; and
menthol at an amount of about 0.05% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.05% to about 15% w/w;
naloxone, a pharmaceutically acceptable salt thereof or a derivative thereof at an amount from about 0.005% to about 5% w/w;
water as a solvent in an amount from about 10% w/w to about 95% w/w;
a cosolvent consisting of a mixture of an alcohol in an amount from about 10% to about 80% w/w and a glycol in an amount from about 0.5% w/w to about 50% w/w;
an antioxidant in an amount from about 0.001% to about 0.2% w/w; and
a chelating agent in an amount from about 0.001% to about 0.1% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.05% to about 10% w/w;
naloxone, a pharmaceutically acceptable salt thereof or a derivative thereof at an amount from about 0.1% to about 3% w/w;
water as a solvent in an amount from about 20% w/w to about 45% w/w;
a cosolvent consisting of a mixture of ethanol in an amount of 50% w/w to about 60% w/w and propylene glycol in an amount of about 4% w/w to 6% w/w;
an antioxidant selected from a group consisting of butylated hydroxyanisole, butylated hydroxytoluene, methionine, sodium ascorbate, sodium thiosulfate, thioglycerol, cysteine hydrochloride monohydrate, and a mixture thereof at an amount of about 0.01% to about 0.1 w/w;
disodium edetate as a chelating agent at an amount of about 0.001% to about 0.01% w/w; and
menthol at an amount of about 0.005% to 0.5% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.6% to about 10% w/w;
naloxone, a pharmaceutically acceptable salt thereof or a derivative thereof at an amount from about 0.1% to about 3.0% w/w;
menthol at an amount of about 0.05% w/w;
disodium edetate at an amount of about 0.005% w/w;
sodium ascorbate in an amount of about 0.02%;
ethanol in an amount of about 55%;
propylene glycol in an amount from about 5% w/w;
water in an amount from about 25% w/w to 40% w/w;
wherein the % w/w is of the total formulation.

In one embodiment, the present invention is directed to a sublingual spray formulation comprising:
buprenorphine, a pharmaceutically acceptable salt thereof or a derivative thereof at an amount from about 0.05% to about 9.5% w/w;
naloxone, a pharmaceutically acceptable salt thereof or a derivative thereof at an amount from about 0.2% to about 2.7% w/w;
water as a solvent in an amount from about 27.4% w/w to 39.7% w/w;
a cosolvent consisting of a mixture of ethanol in an amount from about 55% w/w and propylene glycol in an amount from about 5% w/w; and
an antioxidant selected from a group consisting of butylated hydroxyanisole, butylated hydroxytoluene, methionine, sodium ascorbate, sodium thiosulfate, thioglycerol, cysteine hydrochloride monohydrate and a mixture thereof in an amount from about 0.001% to about 0.2% w/w.

In one embodiment, the present invention is directed to a sublingual spray formulation comprising:
buprenorphine, a pharmaceutically acceptable salt thereof or a derivative thereof at an amount from about 0.05% to about 9.5% w/w;
naloxone, a pharmaceutically acceptable salt thereof or a derivative thereof at an amount from about 0.005% to about 2.7% w/w;
water as a solvent in an amount from about 27.4% w/w to 39.7% w/w;
a cosolvent consisting of a mixture of ethanol in an amount of about 55% w/w and propylene glycol in an amount of about 5% w/w; and
an antioxidant selected from a group consisting of butylated hydroxyanisole, butylated hydroxytoluene, methionine, sodium ascorbate, sodium thiosulfate, thioglycerol, cysteine hydrochloride monohydrate, and a mixture thereof in an amount from about 0.001% to about 0.2% w/w.

In one embodiment, the present invention is directed to a sublingual spray formulation comprising:
buprenorphine, a pharmaceutically acceptable salt thereof or a derivative thereof at an amount from about 0.05% to about 9.5% w/w;
naloxone, a pharmaceutically acceptable salt thereof or a derivative thereof at an amount from about 0.005% to about 3% w/w;
water as a solvent in an amount from about 27.4% w/w to 39.7% w/w;
a cosolvent consisting of a mixture of ethanol in an amount of about 55% w/w and propylene glycol in an amount of about 5% w/w;
an antioxidant selected from a group consisting of butylated hydroxyanisole, butylated hydroxytoluene, methionine, sodium ascorbate, sodium thiosulfate, thioglycerol, cysteine hydrochloride monohydrate, and a mixture thereof; and
ethylenediaminetetraacetic acid disodium (disodium edetate) as a chelating agent in an amount of about 0.005% w/w or citric acid as a pH adjustor in an amount from about 0.0025 to 10% w/w.

In one embodiment, the sublingual spray formulation comprises:
an amount of buprenorphine of about 0.54% w/w;
an amount of water of about 39.4% w/w;
a cosolvent as a mixture of ethanol in an amount of about 55% w/w and propylene glycol in an amount of about 5% w/w;
an antioxidant as a mixture of BHA in an amount of about 0.01% w/w and BHT in an amount of about 0.005% w/w; and
menthol as a permeation enhancer in an amount of about 0.05% w/w.

In one embodiment, the sublingual spray formulation comprises:
an amount of buprenorphine of about 0.54% w/w;
an amount of water of about 39.4% w/w;
a cosolvent as a mixture of ethanol in an amount of about 55% w/w and propylene glycol in an amount of about 5% w/w;
sodium thiosulfate as an antioxidant in an amount of about 0.01% w/w;
menthol as a permeation enhancer in an amount of about 0.05% w/w; and
citric acid as a pH adjustor in an amount of about 0.002% w/w.

In one embodiment, the sublingual spray formulation comprises:
an amount of buprenorphine of about 0.54% w/w;
an amount of water of about 39.39% w/w;
a cosolvent as a mixture of ethanol in an amount of about 55% w/w and propylene glycol in an amount of about 5% w/w;
sodium ascorbate as an antioxidant in an amount of about 0.01% w/w;
menthol as a permeation enhancer in an amount of about 0.05% w/w; and
disodium edetate as a chelating agent in an amount of about 0.01% w/w.

In one embodiment, the sublingual spray formulation comprises:
an amount of buprenorphine of about 0.54% w/w;
an amount of water of about 39.45% w/w;
a cosolvent as a mixture of ethanol in an amount of about 55% w/w and propylene glycol in an amount of about 5% w/w; and
BHA as an antioxidant in an amount of about 0.01% w/w.

In one embodiment, the sublingual spray formulation comprises:

an amount of buprenorphine of about 8.602% w/w;

an amount of naloxone of about 2.44% w/w;

an amount of water of about 29% w/w;

an amount of sodium thiosulfate of about 0.01% w/w; and

an amount of citric acid of about 0.0025% w/w.

In one embodiment, the sublingual spray formulation comprises:

an amount of buprenorphine of about 8.602% w/w;

an amount of naloxone of about 2.44% w/w;

an amount of water of about 29% w/w;

an amount of sodium thiosulfate of about 0.01% w/w; and

an amount of disodium edetate of about 0.005% w/w.

In one embodiment, the sublingual spray formulation comprises:
an amount of buprenorphine of about 8.602% w/w;
an amount of naloxone of about 2.44% w/w;
an amount of water of about 29% w/w;
an antioxidant as a mixture of BHA in an amount of about 0.01% w/w and BHT in an amount of about 0.005% w/w; and
an amount of disodium edetate of about 0.005% w/w.

In one embodiment, the sublingual spray formulation comprises:

an amount of buprenorphine of about 8.602% w/w;

an amount of naloxone of about 2.44% w/w;

an amount of water of about 29% w/w;

an amount of sodium ascorbate of about 0.02% w/w; and

an amount of disodium edetate of about 0.005% w/w.

In one embodiment, the sublingual spray formulation comprises:

an amount of buprenorphine of about 8.39% w/w;

an amount of naloxone of about 2.37% w/w;

an amount of water of about 29% w/w;

an amount of ethanol of about 55% w/w;

an amount of propylene glycol of about 5% w/w;

an amount of sodium ascorbate of about 0.02% w/w;

an amount of disodium edetate of about 0.005% w/w; and

an amount of menthol of about 0.05% w/w.

In one embodiment, the sublingual spray formulation comprises:

an amount of buprenorphine of about 5.554% w/w;

an amount of naloxone of about 1.57% w/w;

an amount of water of about 33% w/w;

an amount of ethanol of about 55% w/w;

an amount of propylene glycol of about 5% w/w;

an amount of sodium ascorbate of about 0.02% w/w;

an amount of disodium edetate of about 0.005% w/w; and

an amount of menthol of about 0.05% w/w.

In one embodiment, the sublingual spray formulation comprises:

an amount of buprenorphine of about 2.84% w/w;

an amount of naloxone of about 0.804% w/w;

an amount of water of about 36% w/w;

an amount of ethanol of about 55% w/w;

an amount of propylene glycol of about 5% w/w;

an amount of sodium ascorbate of about 0.02% w/w;

an amount of disodium edetate of about 0.005% w/w; and

an amount of menthol of about 0.05% w/w.

In one embodiment, the sublingual spray formulation comprises:

an amount of buprenorphine of about 1.42% w/w;

an amount of naloxone of about 0.402% w/w;

an amount of water of about 38% w/w;

an amount of ethanol of about 55% w/w;

an amount of propylene glycol of about 5% w/w;

an amount of sodium ascorbate of about 0.02% w/w;

an amount of disodium edetate of about 0.005% w/w; and

an amount of menthol of about 0.05% w/w.

In one embodiment, the sublingual spray formulation comprises:
an amount of buprenorphine from about 0.813% to about 1.3% w/w, preferably 0.0813% w/w, 0.1625% w/w, 0.325% w/w, 0.65% w/w or 1.3% w/w;
an amount of BHA of about 0.01% w/w;
an amount of BHT of about 0.005% w/w;
an amount of ethanol of about 55% w/w;
an amount of propylene glycol of about 5% w/w; and
an amount of water from about 39.8537% to about 38.635% w/w, preferably 39.8537% w/w, 39.7725% w/w, 39.61% w/w, 39.285% w/w or 38.635% w/w.

In one embodiment, the spray formulation comprises:
about 5.167% w/w buprenorphine or a pharmaceutically acceptable salt thereof;
about 0.878% w/w naloxone or a pharmaceutically acceptable salt thereof;
about 0.050% w/w L-menthol;
about 0.020% w/w sodium ascorbate;
about 0.005% w/w edetate disodium dihydrate;
about 55% w/w ethanol;
about 5% w/w propylene glycol; and
about 33.88% w/w water.

In one embodiment, the spray formulation comprises:
about 1.302% w/w buprenorphine or a pharmaceutically acceptable salt thereof;
about 0.221% w/w naloxone or a pharmaceutically acceptable salt thereof;
about 0.050% w/w L-menthol;
about 0.020% w/w sodium ascorbate;
about 0.005% w/w edetate disodium dihydrate;
about 55% w/w ethanol;
about 5% w/w propylene glycol; and
about 38.402% w/w water.

The following examples are intended to illustrate the present invention and to teach one of ordinary skill in the art how to make and use the invention. They are not intended to be limiting in any way.

### EXAMPLES

### Example 1: Stable Buprenorphine Formulations

Method of Making the Formulations

Sublingual spray formulations were created by first degassing ethanol and USP purified water, separately. Next, the ethanol and purified water were each purged with nitrogen. Soluble excipients were then dissolved in either the ethanol or the purified water based on their solubility. Next, the solutions were combined. Active pharmaceutical ingredient/s was/were added to the final solution and mixed until dissolved.

Formulations

TABLE 1
Stable Sublingual Buprenorphine Spray Formulations

| Formulation | Control | #1 | #2 | #3 | #4 | #5 | #6 | #7 | #8 | #9 |
|---|---|---|---|---|---|---|---|---|---|---|
| Buprenorphine HCl | 0.538 | 0.538 | 0.538 | 0.538 | 0.538 | 0.538 | 0.538 | 0.538 | 0.538 | 0.538 |
| Water (USP) | 39.462 | 39.452 | 39.397 | 39.372 | 89.427 | 94.427 | 39.39 | 39.4 | 39.405 | 69.472 |
| Ethanol | 55 | 55 | 55 | 55 | 10 | | 55 | 55 | 55 | |
| Propylene Glycol | 5 | 5 | 5 | 5 | | 5 | 5 | 5 | 5 | |
| HPβCD | | | | | | | | | | 30 |
| BHA | | 0.01 | 0.01 | | | | | | | |
| BHT | | | 0.005 | | | | | | | |
| Sodium Ascorbate | | | | 0.02 | 0.02 | 0.02 | 0.01 | | | 0.02 |
| Sodium Thiosulfate | | | | | | | | 0.01 | | |
| Methionine | | | | | | | | | 0.005 | |
| Menthol | | | 0.05 | 0.05 | | | 0.05 | 0.05 | 0.05 | |
| Citric Acid | | | | 0.02 | 0.015 | 0.015 | | 0.002 | 0.002 | |
| Disodium Edetate | | | | | | | 0.01 | | | |
| pH | 5.09 | 4.99 | 5.11 | 4.71 | 4.01 | 4 | 4.43 | 3.9 | 3.85 | No Data |

values = % w/w

Stability Data

The formulations listed in Table 1 were subject to stability test at 40° C.±2° C. under 75%±5% relative humidity for six months. Stability data was collected at zero, and six months. Assay and impurities were detected using high performance liquid chromatography with an ultraviolet detector. The assay was performed at 288 nm and indicated as a % of initial concentration. For all impurities, analysis was performed at 240 nm and expressed as a % area. Amounts of particular impurities are listed in Table 2 as a percentage of the area of each formulation along with amount of total impurities.

TABLE 2
Stability Data for Sublingual Buprenorphine Spray Formulations stored at 40°
C. ± 2° C. under 75% ± 5% relative humidity.

| | Control | | #1 | | #2 | | #3 | | #4 | |
|---|---|---|---|---|---|---|---|---|---|---|
| Time (m) | 0 | 6 | 0 | 6 | 0 | 6 | 0 | 6 | 0 | 6 |
| Assay | 100 | 104 | 100 | 104.2 | 100 | 104.1 | 100 | 103.3 | 100 | 102.7 |
| A | BQL | ND | BQL | ND | ND | ND | BQL | ND | ND | ND |
| B | ND | 0.27 | ND | 0.09 | ND | 0.06 | ND | 0.21 | ND | 0.05 |
| D | ND | BQL | ND | ND | ND | ND | ND | ND | ND | ND |
| G | BQL | 0.64 | ND | 0.06 | ND | BQL | ND | 0.11 | 0.11 | 0.68 |
| H | ND | ND | ND | ND | ND | ND | ND | ND | ND | ND |
| Bisalkyl- | ND | ND | ND | 0.31 | ND | BQL | ND | ND | ND | ND |
| buprenorphine | | | | | | | | | | |
| Unspecified | BQL | ND | ND | ND | ND | ND | ND | ND | ND | ND |
| Total | 0 | 0.91 | 0 | 0.46 | 0 | 0.06 | 0 | 0.32 | 0.11 | 0.73 |
| (% area) | | | | | | | | | | |

| | #5 | | #6 | | #7 | | #8 | | #9 | |
|---|---|---|---|---|---|---|---|---|---|---|
| Time (m) | 0 | 6 | 0 | 6 | 0 | 6 | 0 | 6 | 0 | 6 |
| Assay | 100 | 99.2 | 100 | 99.3 | 100 | 99.6 | 100 | 98.2 | 100 | 101.8 |
| A | ND | ND | ND | 0.06 | ND | BQL | ND | 0.05 | ND | ND |
| B | ND | 0.09 | ND | 0.17 | ND | 0.08 | ND | 0.2 | ND | BQL |
| D | ND | ND | ND | ND | ND | ND | ND | ND | ND | ND |
| G | 0.09 | 0.77 | ND | 0.07 | ND | ND | ND | 0.34 | ND | 0.4 |
| H | ND | ND | ND | 0.08 | ND | ND | ND | ND | ND | BQL |
| Bisalkyl- | ND | ND | ND | 0.05 | ND | ND | ND | ND | ND | ND |
| buprenorphine | | | | | | | | | | |
| Unspecified | ND | 0.06 | BQL | ND | 0.05 | 0.08 | 0.06 | 0.21 | ND | ND |
| Total | 0.09 | 0.92 | 0 | 0.43 | 0.05 | 0.16 | 0.06 | 0.8 | 0 | 0.4 |
| (% area) | | | | | | | | | | |

BQL = Below Quantifiable Limit;
ND = Not Detected

Sublingual buprenorphine spray formulations contained less than one percent total impurities after six months at 40° C. Control and formulations 1, 3, 4, 5, 6, 8 and 9 showed significant increase in levels of individual impurities (impurity B, impurity G, bisalkyl or unspecified impurity) at the 6 month time point whereas formulations containing BHA and BHT (#2) or sodium thiosulfate (#7) showed good stability. pH also played a role in the stability of the product. These results represent sublingual buprenorphine spray formulations that would remain stable for two years at room temperature.

### Example 2: Stable Buprenorphine/Naloxone Formulations

Method of Making the Formulations

Sublingual spray formulations were created by first degassing ethanol and USP purified water, separately. Next, the ethanol and purified water were each purged with nitrogen. Soluble excipients were then dissolved in either the ethanol or the purified water based on their solubility. Next, the solutions were combined. Buprenorphine and naloxone were added to the final solution and mixed until dissolved.

### Formulations

TABLE 3
Stable Buprenorphine/Naloxone Sublingual Spray Formulations

| Formulation | Control #2 | #10 | #11 | #12 | #13 |
|---|---|---|---|---|---|
| Buprenorphine HCl | 8.602 | 8.602 | 8.602 | 8.602 | 8.602 |
| Naloxone HCl | 2.44 | 2.44 | 2.44 | 2.44 | 2.44 |
| Water (USP) | 28.958 | 28.9455 | 28.943 | 28.938 | 28.933 |
| Ethanol | 55 | 55 | 55 | 55 | 55 |
| Propylene Glycol | 5 | 5 | 5 | 5 | 5 |
| BHA | | | | 0.01 | |
| BHT | | | | 0.005 | |
| Sodium Ascorbate | | | | | 0.02 |
| Sodium Thiosulfate | | 0.01 | 0.01 | | |
| Citric Acid | | 0.0025 | | | |
| Disodium Edetate | | | 0.005 | 0.005 | 0.005 |

values = % w/w

### Stability Data

The formulations listed in Table 3 were subject to stability test at 40° C.±2° C. under 75%±5% relative humidity for three months and at ±25° C. under 60%±5% relative humidity for three months. Stability data was collected at zero, one, two and three months at 40° C. and at zero, one and three months at 25° C. Assay and impurities were detected using high performance liquid chromatography with an ultraviolet detector. Buprenorphine assay was performed at 288 nm and indicated as a % of initial concentration. For all buprenorphine impurities, analysis was performed at 240 nm and expressed as a % area. Naloxone assay was performed at 280 nm and indicated as a % of initial concentration and for all naloxone impurities, analysis was performed at 230 nm. Amounts of particular impurities are listed in Tables 4 and 5 for 40° C. and in Table 6 for 25° C. as a percentage of the area of each formulation along with amount of total impurities. Relative retention time (“RRT”) is given for each impurity.

TABLE 4
Stability Data for Control #2 stored at 40° C. ± 2° C./75% ± 5% relative humidity for 1, 2 and 3 months.

| 40° C. | Control #2 | | | | | 40° C. | Control #2 | | | | |
|---|---|---|---|---|---|---|---|---|---|---|---|
| Buprenorphine | RRT | 0 m | 1 m | 2 m | 3 m | Naloxone | RRT | 0 m | 1 m | 2 m | 3 m |
| Assay | | 100% | 96.93% | 94.22% | 94.27% | Assay | | 100% | 96.31% | 97.22% | 95.62% |
| Impurity B | 0.4 | ND | ND | 0.09% | 0.12% | Impurity C | 0.66 | ND | 1.11% | 1.71% | 2.02% |
| Impurity J | 1.1 | ND | ND | BQL | BQL | Impurity A | 0.83 | ND | ND | 0.10% | 0.19% |
| Impurity F | 1.27 | ND | ND | BQL | BQL | Impurity E | 2.85 | ND | ND | 0.09% | ND |
| Impurity G | 1.8 | 0.11% | 1.84% | 3.10% | 4.14% | Impurity D | 0.20 | ND | ND | ND | 0.09% |
| Unknown | 0.26 | ND | ND | ND | BQL | Unknown | 0.28 | ND | 0.09% | 0.17% | 0.23% |
| Impurities | 0.86 | ND | 0.28% | 0.46% | 0.63% | Impurities | 0.30 | ND | ND | 0.09% | 0.17% |
| | 2.15 | ND | 0.23% | 0.33% | 0.42% | | 0.47 | ND | ND | ND | 0.06% |
| Total (% area) | | 0.11% | 2.35% | 3.98% | 5.31% | | 0.52 | ND | 0.34% | 0.73% | 1.17% |
| | | | | | | | 4.30 | ND | ND | ND | 0.33% |
| | | | | | | Total (% area) | | 0.00% | 1.54% | 2.89% | 4.26% |

BQL = Below Qantifiable Limit;
ND = Not Detected

The control formulation for the buprenorphine/naloxone sublingual spray formulation contained greater than 1% impurities of both buprenorphine and naloxone within one month at 40° C. and between about 4% and about 5% at three months.

TABLE 5
Stability Data for Buprenorphine/Naloxone Sublingual Spray Formulations stored
at 40° C. ± 2° C./75% ± 5% relative humidity for 1, 2 and 3 months.

| 40° C. | #10 | | | | | #11 | | | | |
|---|---|---|---|---|---|---|---|---|---|---|
| Buprenorphine | RRT | 0 m | 1 m | 2 m | 3 m | RRT | 0 m | 1 m | 2 m | 3 m |
| Assay | | 100% | 98.72% | 96.90% | 100.06% | | 100% | 99.26% | 98.91% | 99.96% |
| Impurity G | | | | | | | | | | |
| Total (% area) | | 0.00% | 0.00% | 0.00% | 0.00% | | 0.00% | 0.00% | 0.00% | 0.00% |
| Naloxone | RRT | 0 m | 1 m | 2 m | 3 m | RRT | 0 m | 1 m | 2 m | 3 m |
| Assay | | 100% | 99.19% | 102.69% | 102.42% | | 100% | 99.84% | 102.75% | 102.00% |
| Impurity C | | | | | | | | | | |
| Unknown | | | | | | | | | | |
| Impurities | | | | | | | | | | |
| Total (% area) | | 0.00% | 0.00% | 0.00% | 0.00% | | 0.00% | 0.00% | 0.00% | 0.00% |

| 40° C. | #12 | | | | | #13 | | | | |
|---|---|---|---|---|---|---|---|---|---|---|
| Buprenorphine | RRT | 0 m | 1 m | 2 m | 3 m | RRT | 0 m | 1 m | 2 m | 3 m |
| Assay | | 100 | 99.50% | 101.44% | 101.22% | | 100% | 99.06% | 100.30% | 99.36% |
| Impurity G | 1.8 | ND | ND | ND | 0.05% | | | | | |
| Total (% area) | | 0.00% | 0.00% | 0.00% | 0.05% | | 0.00% | 0.00% | 0.00% | 0.00% |
| Naloxone | RRT | 0 m | 1 m | 2 m | 3 m | RRT | 0 m | 1 m | 2 m | 3 m |
| Assay | | 100% | 97.91% | 102.36% | 103.11% | | 100% | 101.42% | 102.72% | 103.38% |
| Impurity C | 0.66 | ND | ND | 0.11% | 0.14% | 0.66 | ND | ND | ND | 0.09% |
| Unknown | 0.52 | ND | ND | 0.07% | 0.12% | 0.52 | ND | ND | BQL | ND |
| Impurities | 4.02 | ND | ND | ND | ND | | | | | |
| Total (% area) | | 0.00% | 0.00% | 0.18% | 0.26% | | 0.00% | 0.00% | 0.00% | 0.09% |

BQL = Below Qantifiable Limit;
ND = Not Detected

All formulations had less than 1% total impurities at three months. Similar to the buprenorphine only formulations in Example 1, formulations containing sodium thiosulfate (#10 and #11) were exceptionally stable with no impurities after three months. Formulation #12 contains BHA and BHT as the antioxidant and had significant impurities of naloxone (0.26% total impurities). Formulation #13 contains sodium ascorbate and had no impurities of buprenorphine and 0.09% total impurities of naloxone. These results represent sublingual spray formulations that would remain stable for one year at room temperature.

TABLE 6
Stability Data for Buprenorphine/Naloxone Sublingual Spray Formulations stored
at 25° C. ± 2° C./60% ± 5% relative humidity for 1, 2 and 3 months.

| 25° C. | Control #2 | | | | #10 | | | | #11 | | | |
|---|---|---|---|---|---|---|---|---|---|---|---|---|
| Buprenorphine | RRT | 0 m | 1 m | 3 m | RRT | 0 m | 1 m | 3 m | RRT | 0 m | 1 m | 3 m |
| Assay | | 100% | 97.33% | 98.25% | | 100% | 100.14% | 98.82% | | 100% | 100.01% | 99.80% |
| Impurity G | 1.8 | 0.11% | 0.44% | 1.08% | | | | | | | | |
| Unknown | 0.86 | ND | ND | 0.13% | | | | | | | | |
| Impurities | 1.8 | ND | ND | 0.09% | | | | | | | | |
| Total (% area) | | 0.11% | 0.44% | 1.30% | | 0.00% | 0.00% | 0.00% | | 0.00% | 0.00% | 0.00% |
| Naloxone | RRT | 0 m | 1 m | 3 m | RRT | 0 m | 1 m | 3 m | RRT | 0 m | 1 m | 3 m |
| Assay | | 100% | 98.56% | 100.00% | | 100% | 99.08% | 101.67% | | 100% | 99.03% | 102.16% |
| Impurity C | 0.66 | ND | 0.41% | 0.97% | | | | | | | | |
| Impurity A | | | | | | | | | | | | |
| Unknown | 0.28 | ND | ND | 0.08% | | | | | | | | |
| Impurities | 0.52 | ND | ND | 0.13% | | | | | | | | |
| Total (% area) | | 0.00% | 0.41% | 1.18% | | 0.93% | 0.00% | 0.00% | | 0.00% | 0.00% | 0.00% |

| 25° C. | #12 | | | | #13 | | | |
|---|---|---|---|---|---|---|---|---|
| Buprenorphine | RRT | 0 m | 1 m | 3 m | RRT | 0 m | 1 m | 3 m |
| Assay | | 100 | 101.29% | 100.14% | | 100% | 98.37% | 99.74% |
| Impurity G | | | | | | | | |
| Unknown | | | | | | | | |
| Impurities | | | | | | | | |
| Total (% area) | | 0.00% | 0.00% | 0.00% | | 0.00% | 0.00% | 0.00% |
| Naloxone | RRT | 0 m | 1 m | 3 m | RRT | 0 m | 1 m | 3 m |
| Assay | | 100% | 99.03% | 101.77% | | 100% | 100.65% | 102.67% |
| Impurity C | | | | | | | | |
| Impurity A | | | | | 0.83 | ND | ND | 0.11% |
| Unknown | | | | | | | | |
| Impurities | | | | | 0.52 | ND | ND | BQL |
| Total (% area) | | 0.00% | 0.00% | 0.00% | | 0.00% | 0.00% | 0.11% |

BQL = Below Qantifiable Limit;
ND = Not Detected

The control formulation had greater than 1% impurities at three months. All formulations containing antioxidants had less than 1% total impurities at three months. Similar to the buprenorphine only formulations in Example 1, formulations containing sodium thiosulfate (#10 and #11) or a mixture of BHA and BHT (#12) were exceptionally stable with no impurities after three months. Formulation #13 which contains sodium ascorbate had no impurities of buprenorphine and 0.11% total impurities of naloxone after storage at 25° C.±2° C./75%±5% relative humidity.

### Example 3: Pharmacokinetics of Buprenorphine Sublingual Spray Formulations

A study was designed and executed to determine the pharmacokinetics of buprenorphine sublingual spray formulations of the present invention after administration in healthy volunteers under fasting conditions.

The study was a single center, single dose, open-label, 1-sequence, 2-period, ascending dose study design in twelve healthy male and female subjects. The following dose levels of the investigational product were administered under fasting conditions: Dose 1: A single 0.5 mg dose (1 spray of 100 microliters) of Buprenorphine 5 mg/mL Sublingual Spray; and Dose 2: A single 1.0 mg dose (2 sprays of 100 microliters) of Buprenorphine 5 mg/mL Sublingual Spray.

The subjects arrived at the clinical site more than 10 hours before the buprenorphine administration. The subjected were supervised overnight (while fasting) and a single 50 mg dose of naltrexone (1×50 mg tablet) was orally administered with 240 mL of water approximately 1 hour prior to the buprenorphine administration to provide blockade of the pharmacological effects of buprenorphine. Then, a single dose (0.5 mg in period 1 and 1.0 mg in period 2) of the buprenorphine formulation was sublingually administered in the morning. Subjects were allowed to leave the clinical site after the 24-hour post-dose blood draw and returned to the clinical site before the remaining blood sample. The second dose level was administered following favorable safety review. The buprenorphine administrations were separated by a wash-out of 14 calendar days. The parameters are summarized below in Table 7.

TABLE 7
Summary of Pharmacokinetic Parameters

| | Buprenorphine 0.5 mg | | Buprenorphine 1 mg | |
|---|---|---|---|---|
| Parameter | MEAN | C.V. | MEAN | C.V. |
| Cₘₐₓ (ng/mL) | 0.761 | 19.0 | 1.38 | 10.2 |
| ln(Cₘₐₓ) | −0.2904 | −67.1 | 0.3169 | 31.2 |
| Tₘₐₓ (hours) * | 1.75 | 30.8 | 1.50 | 30.6 |
| AUC_{0-T} (ng · h/mL) | 4.37 | 13.6 | 9.12 | 10.7 |
| ln(AUC_{0-T}) | 1.4671 | 9.0 | 2.2053 | 5.0 |
| AUC_{0-∞} (ng · h/mL) | 4.81 | 13.3 | 10.2 | 10.6 |
| ln(AUC_{0-∞}) | 1.5614 | 8.7 | 2.3170 | 4.7 |
| AUC_{0-T/∞} (%) | 91.19 | 6.6 | 89.49 | 3.5 |
| λ_{Z} (hours⁻¹) | 0.0959 | 53.3 | 0.0313 | 17.0 |
| T_{half} (hours) | 9.75 | 57.4 | 22.87 | 20.1 |
| V_{D}/F (L) | 1450 | 54.9 | 3250 | 19.4 |
| Cl/F (L/h) | 106 | 13.8 | 99.1 | 11.2 |
| Cₘₐₓ/D (ng/mL) | 0.761 | 19.0 | 0.690 | 10.2 |
| ln(Cₘₐₓ/D) | −0.2904 | −67.1 | −0.3763 | −26.3 |
| AUC_{0-T}/D (ng · h/mL) | 4.37 | 13.6 | 4.56 | 10.7 |
| ln(AUC_{0-T}/D) | 1.4671 | 9.0 | 1.5122 | 7.3 |
| AUC_{0-∞}/D (ng · h/mL) | 4.81 | 13.3 | 5.10 | 10.6 |
| ln(AUC_{0-∞}/D) | 1.5614 | 8.7 | 1.6238 | 6.7 |

* Tₘₐₓ, the median is presented

As seen in Table 7, the Cmax obtained for buprenorphine were 0.761 ng/mL and 1.38 ng/mL. The Tmax observed for buprenorphine was 1.75 and 1.50 hours following the ascending doses.

### Example 4: Bioavailability of Buprenorphine

A study was designed and executed in order to compare the rate and extent of absorption and bioavailability of 1 mg buprenorphine sublingual spray formulations of the present invention with 0.3 mg (1 mL) Buprenex® (buprenorphine HCl) intramuscular injection and 0.3 mg (1 mL) Buprenex® (buprenorphine HCl) intravenous bolus injection.

This was an open-label, 3-treatment, 3-period, 6-sequence, single-dose, randomized crossover study. Eighteen healthy male and female volunteers were randomly assigned to 1 of 6 treatment sequences. Dosing occurred after an overnight fast and there was a minimum 14-day washout between the dosing in two periods. Blood samples for the measurement of the plasma concentrations of buprenorphine were collected before (pre-dose) and at 5, 10, 20, 30, and 40 minutes and at 1, 1.25, 1.5, 2, 4, 6, 8, 10, 12, 16, 24, 36, 48, 72, 96, 120, and 144 hours after dosing. The results of this study are summarized below in Table 8.

TABLE 8
Bioavailability of Buprenorphine

| Parameter* | Sublingual Spray 1 mg | | Intramuscular 0.3 mg | | Intravenous 0.3 mg | |
|---|---|---|---|---|---|---|
| Cmax (ng/mL) | 1.20 ± 0.507 | (18) | 1.73 ± 1.08 | (18) | 3.95 ± 3.66 | (18) |
| Tmax (h) | 1.50 | (18) | 0.17 | (18) | 0.083 | (18) |
| | [0.50-2.00] | | [0.083-1.50] | | [0.083-0.333] | |
| AUC(0-t) | 7.31 ± 2.80 | (18) | 4.97 ± 0.90 | (18) | 5.09 ± 1.01 | (18) |
| (h × ng/mL) | | | | | | |
| AUC(inf) | 8.19 ± 3.27 | (15) | 5.50 ± 0.83 | (15) | 5.51 ± 1.21 | (17) |
| (h × ng/mL) | | | | | | |
| λz (1/h) | 0.0551 ± 0.0357 | (15) | 0.0655 ± 0.0210 | (15) | 0.1028 ± 0.0641 | (17) |
| t½ (h) | 17.1 ± 8.62 | (15) | 12.0 ± 5.31 | (15) | 9.37 ± 6.49 | (17) |

The absolute bioavailability of buprenorphine, based on AUC(0-t) and AUC(inf), after sublingual administration was 41.03% and 42.57%, respectively.

### Example 5: Buprenorphine Spray Droplet Size Distribution, Spray Pattern and Plume Geometry

A challenge of creating a buprenorphine sublingual spray formulation is that it must be capable of producing spray droplets that are over 10 microns in diameter. Spray droplets 10 microns or smaller could be inhaled into the lungs. The optimal particle size for sublingual spray droplets is from 20 to about 200 microns in diameter. It is desirable for the formulation to have droplet sizes near 20 because this increases the surface area and increased surface area exposure is one factor that contributes to a high bioavailability. Sublingual formulations should be able to maintain a consistent droplet size throughout its shelf life. Applicants found during testing that formulations of the present invention yielded desirable droplet sizes for sublingual administration. The testing also revealed that the formulation dose remains consistent when administered with a spray pump.

Five milligram per mL buprenorphine spray formulations of the present invention were subjected to two different storage conditions (25 and 40 degrees C.) and samples were taken at two different times (5M and 6M) for spray droplet size distribution analysis. Droplet analysis was conducted using standard laser analysis procedures known by those of skill in the art.

Droplet size distribution (Dv10, Dv50, Dv90, percent droplets less than 10 micrometers in diameter, D(4,3) and Span tested at two distances, 3 cm and 6 cm for upright and horizontal samples stored at 25 and 40 degrees C.) and spray pattern (Dmin, Dmax and ovality ratio tested at two distances, 3 cm and 6 cm for upright and horizontal samples stored at 25 and 40 degrees C.) were determined. D(4,3) refers to the volume moment mean of the particles; Dv10 refers to droplet size for which 10% of the total volume is obtained; Dv50 refers to droplet size for which 50% of the total volume is obtained; Dv90 refers to droplet size for which 90% of the total volume is obtained; Span refers to distribution span (Dv90−Dv10)/Dv50; DSD refers to droplet size distribution; the temperature listed is the storage temperature; U refers to an upright position of the spray pump; and H refers to horizontal position of the spray pump. The results of these studies can be seen below in Tables 9 to 40.

In addition, the formulations were tested for plume geometry including width and angle using standard procedures known by those of skill in the art. This testing showed that the spray pattern and plume were acceptable for formulations of the present invention. The results of these studies can be seen below in Tables 41 and 42.

TABLE 9
Droplet Size Distribution at 3 cm for sample stored
at 25 degrees C., Upright position, 5 M

| | DSD 3 cm | Dv(10) | Dv(50) | Dv(90) | | D(4,3) | |
|---|---|---|---|---|---|---|---|
| | 25° C. - U | (μm) | (μm) | (μm) | % < 10μ | (μm) | Span |
| | Mean | 25.37 | 53.25 | 111.1 | 0.9507 | 62.07 | 1.609 |
| Range | Min | 24.38 | 51.44 | 106.0 | 0.8534 | 59.51 | 1.539 |
| | Max | 26.20 | 55.85 | 119.4 | 1.0410 | 65.72 | 1.705 |

TABLE 10
Droplet Size Distribution at 6 cm for sample stored
at 25 degrees C., Upright position, 5 M

| | DSD 6 cm | Dv(10) | Dv(50) | Dv(90) | | D(4,3) | |
|---|---|---|---|---|---|---|---|
| | 25° C. - U | (μm) | (μm) | (μm) | % < 10μ | (μm) | Span |
| | Mean | 30.58 | 56.68 | 102.7 | 1.5794 | 62.37 | 1.270 |
| Range | Min | 28.93 | 52.00 | 90.5 | 1.4610 | 56.45 | 1.171 |
| | Max | 31.60 | 60.47 | 113.4 | 1.7840 | 67.41 | 1.355 |

TABLE 11
Droplet Size Distribution at 3 cm for sample stored
at 25 degrees C., Horizontal position, 5 M

| | DSD 3 cm | Dv(10) | Dv(50) | Dv(90) | | D(4,3) | |
|---|---|---|---|---|---|---|---|
| | 25° C. - H | (μm) | (μm) | (μm) | % < 10μ | (μm) | Span |
| | Mean | 24.65 | 53.78 | 138.2 | 0.7813 | 72.37 | 2.123 |
| Range | Min | 21.87 | 50.76 | 105.8 | 0.0000 | 59.42 | 1.593 |
| | Max | 26.70 | 58.10 | 194.5 | 1.1560 | 89.39 | 3.295 |

TABLE 12
Droplet Size Distribution at 6 cm for sample stored
at 25 degrees C., Horizontal position, 5 M

| | DSD 6 cm | Dv(10) | Dv(50) | Dv(90) | | D(4,3) | |
|---|---|---|---|---|---|---|---|
| | 25° C. - H | (μm) | (μm) | (μm) | % < 10μ | (μm) | Span |
| | Mean | 30.18 | 55.86 | 108.3 | 0.8612 | 68.69 | 1.403 |
| Range | Min | 26.86 | 52.98 | 96.1 | 0.0637 | 63.28 | 1.171 |
| | Max | 32.03 | 59.90 | 124.7 | 1.6630 | 74.75 | 1.782 |

TABLE 13
Droplet Size Distribution at 3 cm for sample stored
at 40 degrees C., Upright position, 5 M

| | DSD 3 cm | Dv(10) | Dv(50) | Dv(90) | | D(4,3) | |
|---|---|---|---|---|---|---|---|
| | 40° C. - U | (μm) | (μm) | (μm) | % < 10μ | (μm) | Span |
| | Mean | 26.75 | 56.64 | 120.3 | 0.9120 | 66.53 | 1.651 |
| Range | Min | 26.22 | 55.44 | 116.8 | 0.7907 | 65.09 | 1.612 |
| | Max | 27.33 | 58.02 | 122.7 | 0.9900 | 67.94 | 1.689 |

TABLE 14
Droplet Size Distribution at 6 cm for sample stored
at 40 degrees C., Upright position, 5 M

| | DSD 6 cm | Dv(10) | Dv(50) | Dv(90) | | D(4,3) | |
|---|---|---|---|---|---|---|---|
| | 40° C. - U | (μm) | (μm) | (μm) | % < 10μ | (μm) | Span |
| | Mean | 32.87 | 63.39 | 121.7 | 1.3128 | 71.44 | 1.390 |
| Range | Min | 31.62 | 59.93 | 111.7 | 0.6002 | 66.68 | 1.280 |
| | Max | 35.85 | 79.44 | 174.7 | 1.5100 | 94.26 | 1.748 |

TABLE 15
Droplet Size Distribution at 3 cm for sample stored
at 40 degrees C., Horizontal position, 5 M

| | DSD 3 cm | Dv(10) | Dv(50) | Dv(90) | | D(4,3) | |
|---|---|---|---|---|---|---|---|
| | 40° C. - H | (μm) | (μm) | (μm) | % < 10μ | (μm) | Span |
| | Mean | 26.08 | 55.51 | 116.1 | 0.8906 | 64.59 | 1.619 |
| Range | Min | 24.86 | 51.65 | 104.2 | 0.7230 | 59.27 | 1.530 |
| | Max | 27.12 | 58.59 | 126.6 | 1.0880 | 69.05 | 1.710 |

TABLE 16
Droplet Size Distribution at 6 cm for sample stored
at 40 degrees C., Horizontal position, 5 M

| | DSD 6 cm | Dv(10) | Dv(50) | Dv(90) | | D(4,3) | |
|---|---|---|---|---|---|---|---|
| | 40° C. - H | (μm) | (μm) | (μm) | % < 10μ | (μm) | Span |
| | Mean | 30.96 | 57.88 | 105.6 | 1.5678 | 63.84 | 1.288 |
| Range | Min | 29.43 | 54.51 | 97.5 | 1.1350 | 59.57 | 1.195 |
| | Max | 31.84 | 62.23 | 120.3 | 1.7230 | 70.09 | 1.429 |

TABLE 17
Plume Geometry at 3 cm for sample stored
at 40 degrees C., Upright position, 5 M

| | Spray Pattern 3 cm | Dmin | Dmax | Ovality |
|---|---|---|---|---|
| | 40° C. - U | (mm) | (mm) | Ratio |
| | Mean | 12.8 | 20.0 | 1.584 |
| Range | Min | 11.6 | 17.2 | 1.289 |
| | Max | 13.6 | 24.7 | 2.043 |

TABLE 18
Plume Geometry at 6 cm for sample stored at
25 degrees C., Horizontal position, 5 M

| | Spray Pattern 6 cm | Dmin | Dmax | Ovality |
|---|---|---|---|---|
| | 25° C. - H | (mm) | (mm) | Ratio |
| | Mean | 21.4 | 29.1 | 1.362 |
| Range | Min | 20.2 | 27.1 | 1.228 |
| | Max | 22.5 | 32.0 | 1.511 |

TABLE 19
Plume Geometry at 3 cm for sample stored at
25 degrees C., Horizontal position, 5 M

| | Spray Pattern 3 cm | Dmin | Dmax | Ovality |
|---|---|---|---|---|
| | 25° C. - H | (mm) | (mm) | Ratio |
| | Mean | 13.6 | 19.5 | 1.436 |
| Range | Min | 13.0 | 18.0 | 1.382 |
| | Max | 14.2 | 21.1 | 1.580 |

TABLE 20
Plume Geometry at 6 cm for sample stored
at 25 degrees C., Upright position, 5 M

| | Spray Pattern 6 cm | Dmin | Dmax | Ovality |
|---|---|---|---|---|
| | 25° C. - U | (mm) | (mm) | Ratio |
| | Mean | 21.3 | 30.1 | 1.421 |
| Range | Min | 19.9 | 26.7 | 1.244 |
| | Max | 22.3 | 33.4 | 1.679 |

TABLE 21
Plume Geometry at 3 cm for sample stored
at 25 degrees C., Upright position, 5 M

| | Spray Pattern 3 cm | Dmin | Dmax | Ovality |
|---|---|---|---|---|
| | 25° C. - U | (mm) | (mm) | Ratio |
| | Mean | 14.4 | 19.1 | 1.320 |
| Range | Min | 13.2 | 17.1 | 1.212 |
| | Max | 15.9 | 22.3 | 1.426 |

TABLE 22
Plume Geometry at 3 cm for sample stored at
40 degrees C., Horizontal position, 5 M

| | Spray Pattern 3 cm | Dmin | Dmax | Ovality |
|---|---|---|---|---|
| | 40° C. - H | (mm) | (mm) | Ratio |
| | Mean | 13.0 | 18.3 | 1.415 |
| Range | Min | 12.3 | 16.1 | 1.180 |
| | Max | 13.9 | 21.3 | 1.662 |

TABLE 23
Plume Geometry at 6 cm for sample stored
at 40 degrees C., Upright position, 5 M

| | Spray Pattern 6 cm | Dmin | Dmax | Ovality |
|---|---|---|---|---|
| | 40° C. - U | (mm) | (mm) | Ratio |
| | Mean | 20.8 | 32.2 | 1.578 |
| Range | Min | 18.3 | 25.3 | 1.151 |
| | Max | 22.2 | 43.2 | 2.317 |

TABLE 24
Plume Geometry at 6 cm for sample stored at 40 degrees C.,
Horizontal position, 5M

| | Spray Pattern 6 cm | Dmin | Dmax | Ovality |
|---|---|---|---|---|
| | 40° C.—H | (mm) | (mm) | Ratio |
| | Mean | 21.5 | 29.4 | 1.371 |
| Range | Min | 19.8 | 27.1 | 1.253 |
| | Max | 23.3 | 32.5 | 1.639 |

TABLE 25
Droplet Size Distribution at 3 cm for sample stored
at 25 degrees C., Upright position, 6 M

| | DSD 3 cm | Dv(10) | Dv(50) | Dv(90) | | D(4,3) | |
|---|---|---|---|---|---|---|---|
| | 25° C. - U | (μm) | (μm) | (μm) | % < 10μ | (μm) | Span |
| | Mean | 26.22 | 57.53 | 121.8 | 0.5523 | 67.25 | 1.652 |
| Range | Min | 24.63 | 50.98 | 104.4 | 0.0000 | 59.18 | 1.544 |
| | Max | 27.73 | 68.01 | 148.6 | 0.9883 | 79.42 | 1.783 |

TABLE 26
Droplet Size Distribution at 6 cm for sample stored
at 25 degrees C., Upright position, 6 M

| | DSD 6 cm | Dv(10) | Dv(50) | Dv(90) | | D(4,3) | |
|---|---|---|---|---|---|---|---|
| | 25° C. - U | (μm) | (μm) | (μm) | % < 10μ | (μm) | Span |
| | Mean | 31.87 | 62.59 | 119.9 | 1.1915 | 70.21 | 1.405 |
| Range | Min | 29.24 | 58.74 | 111.6 | 0.8993 | 65.79 | 1.282 |
| | Max | 33.93 | 66.29 | 133.7 | 1.4090 | 75.92 | 1.528 |

TABLE 27
Droplet Size Distribution at 3 cm for sample stored
at 25 degrees C., Horizontal position, 6 M

| | DSD 3 cm | Dv(10) | Dv(50) | Dv(90) | | D(4,3) | |
|---|---|---|---|---|---|---|---|
| | 25° C. - H | (μm) | (μm) | (μm) | % < 10μ | (μm) | Span |
| | Mean | 24.55 | 50.03 | 101.6 | 0.8918 | 57.62 | 1.538 |
| Range | Min | 22.88 | 46.53 | 91.7 | 0.0000 | 52.75 | 1.476 |
| | Max | 25.64 | 52.39 | 109.5 | 1.3350 | 61.24 | 1.633 |

TABLE 28
Droplet Size Distribution at 6 cm for sample stored
at 25 degrees C., Horizontal position, 6 M

| | DSD 6 cm | Dv(10) | Dv(50) | Dv(90) | | D(4,3) | |
|---|---|---|---|---|---|---|---|
| | 25° C. - H | (μm) | (μm) | (μm) | % < 10μ | (μm) | Span |
| | Mean | 29.58 | 56.85 | 105.2 | 1.3818 | 62.82 | 1.323 |
| Range | Min | 28.53 | 51.57 | 89.4 | 1.0870 | 55.73 | 1.178 |
| | Max | 30.75 | 60.69 | 116.4 | 1.6780 | 67.86 | 1.434 |

TABLE 29
Droplet Size Distribution at 3 cm for sample stored
at 40 degrees C., Upright position, 6 M

| | DSD 3 cm | Dv(10) | Dv(50) | Dv(90) | | D(4,3) | |
|---|---|---|---|---|---|---|---|
| | 40° C. - U | (μm) | (μm) | (μm) | % < 10μ | (μm) | Span |
| | Mean | 27.60 | 58.79 | 125.9 | 0.4862 | 69.31 | 1.669 |
| Range | Min | 26.50 | 52.85 | 111.3 | 0.0000 | 62.36 | 1.579 |
| | Max | 29.11 | 65.51 | 140.0 | 0.7686 | 76.44 | 1.729 |

TABLE 30
Droplet Size Distribution at 6 cm for sample stored
at 40 degrees C., Upright position, 6 M

| | DSD 6 cm | Dv(10) | Dv(50) | Dv(90) | | D(4,3) | |
|---|---|---|---|---|---|---|---|
| | 40° C. - U | (μm) | (μm) | (μm) | % < 10μ | (μm) | Span |
| | Mean | 33.68 | 67.20 | 131.3 | 1.0200 | 76.03 | 1.450 |
| Range | Min | 32.54 | 63.80 | 118.0 | 0.8835 | 70.69 | 1.314 |
| | Max | 35.01 | 70.75 | 141.2 | 1.4480 | 80.26 | 1.543 |

TABLE 31
Droplet Size Distribution at 3 cm for sample stored
at 40 degrees C., Horizontal position, 6 M

| | DSD 3 cm | Dv(10) | Dv(50) | Dv(90) | | D(4,3) | |
|---|---|---|---|---|---|---|---|
| | 40° C. - H | (μm) | (μm) | (μm) | % < 10μ | (μm) | Span |
| | Mean | 27.75 | 55.42 | 114.3 | 0.0005 | 64.60 | 1.559 |
| Range | Min | 26.47 | 52.01 | 104.6 | 0.0000 | 60.13 | 1.475 |
| | Max | 29.22 | 59.01 | 124.9 | 0.0019 | 69.62 | 1.621 |

TABLE 32
Droplet Size Distribution at 6 cm for sample stored
at 40 degrees C., Horizontal position, 6 M

| | DSD 6 cm | Dv(10) | Dv(50) | Dv(90) | | D(4,3) | |
|---|---|---|---|---|---|---|---|
| | 40° C. - H | (μm) | (μm) | (μm) | % < 10μ | (μm) | Span |
| | Mean | 34.33 | 63.86 | 118.0 | 0.9685 | 70.95 | 1.309 |
| Range | Min | 32.47 | 60.19 | 110.1 | 0.0624 | 66.54 | 1.251 |
| | Max | 37.21 | 68.17 | 129.6 | 1.5090 | 76.88 | 1.363 |

TABLE 33
Plume Geometry at 3 cm for sample stored at 25 degrees
C., Upright position, 6M

| | Spray Pattern 3 cm | Dmin | Dmax | Ovality |
|---|---|---|---|---|
| | 25° C.—U | (mm) | (mm) | Ratio |
| | Mean | 14.0 | 20.8 | 1.489 |
| Range | Min | 13.4 | 17.9 | 1.300 |
| | Max | 14.5 | 23.1 | 1.664 |

TABLE 34
Plume Geometry at 6 cm for sample stored at 25 degrees C.,
Upright position, 6M

| | Spray Pattern 6 cm | Dmin | Dmax | Ovality |
|---|---|---|---|---|
| | 25° C.—U | (mm) | (mm) | Ratio |
| | Mean | 20.3 | 30.3 | 1.497 |
| Range | Min | 19.1 | 27.4 | 1.320 |
| | Max | 21.1 | 33.6 | 1.705 |

TABLE 35
Plume Geometry at 3 cm for sample stored at 25 degrees C.,
Horizontal position, 6M

| | Spray Pattern 3 cm | Dmin | Dmax | Ovality |
|---|---|---|---|---|
| | 25° C.—H | (min) | (mm) | Ratio |
| | Mean | 14.0 | 21.4 | 1.549 |
| Range | Min | 12.9 | 19.8 | 1.276 |
| | Max | 15.7 | 23.9 | 1.852 |

TABLE 36
Plume Geometry at 6 cm for sample stored at 25 degrees C.,
Horizontal position, 6M

| | Spray Pattern 6 cm | Dmin | Dmax | Ovality |
|---|---|---|---|---|
| | 25° C.—H | (mm) | (mm) | Ratio |
| | Mean | 20.2 | 32.3 | 1.599 |
| Range | Min | 18.8 | 28.4 | 1.390 |
| | Max | 21.3 | 37.7 | 1.808 |

TABLE 37
Plume Geometry at 3 cm for sample stored at 40 degrees C.,
Upright position, 6M

| | Spray Pattern 3 cm | Dmin | Dmax | Ovality |
|---|---|---|---|---|
| | 40° C.—U | (mm) | (mm) | Ratio |
| | Mean | 14.9 | 19.2 | 1.284 |
| Range | Min | 13.8 | 17.3 | 1.155 |
| | Max | 15.5 | 20.8 | 1.399 |

TABLE 38
Plume Geometry at 6 cm for sample stored at 40 degrees
C., Upright position, 6M

| | Spray Pattern 6 cm | Dmin | Dmax | Ovality |
|---|---|---|---|---|
| | 40° C.—U | (mm) | (mm) | Ratio |
| | Mean | 21.3 | 27.5 | 1.296 |
| Range | Min | 19.8 | 26.5 | 1.194 |
| | Max | 22.8 | 29.3 | 1.427 |

TABLE 39
Plume Geometry at 3 cm for sample stored at 40 degrees C.,
Horizontal position, 6M

| | Spray Pattern 3 cm | Dmin | Dmax | Ovality |
|---|---|---|---|---|
| | 40° C.—H | (mm) | (mm) | Ratio |
| | Mean | 14.6 | 22.5 | 1.547 |
| Range | Min | 13.9 | 20.8 | 1.430 |
| | Max | 16.0 | 24.8 | 1.781 |

TABLE 40
Plume Geometry at 6 cm for sample stored at 40 degrees C.,
Horizontal position, 6M

| | Spray Pattern 6 cm | Dmin | Dmax | Ovality |
|---|---|---|---|---|
| | 40° C.—H | (mm) | (mm) | Ratio |
| | Mean | 21.5 | 29.4 | 1.371 |
| Range | Min | 19.8 | 27.1 | 1.253 |
| | Max | 23.3 | 32.5 | 1.639 |

TABLE 41
Plume Geometry at 3 cm (width and angle)

| | 3 cm | Width (mm) | Angle (°) |
|---|---|---|---|
| | Mean | 27.9 | 49.9 |
| Range | Min | 25.5 | 46.1 |
| | Max | 30.8 | 54.3 |

TABLE 42
Plume Geometry at 6 cm (width and angle)

| | 6 cm | Width (mm) | Angle (°) |
|---|---|---|---|
| | Mean | 40.2 | 37.0 |
| Range | Min | 36.0 | 33.4 |
| | Max | 43.9 | 40.2 |

### Example 6. Further Buprenorphine Formulations

TABLE 43
Further Buprenorphine Formulations

| | Formulation | | | | |
|---|---|---|---|---|---|
| | #14 | #15 | #16 | #17 | #18 |
| Buprenorphine HCl | 0.0813 | 0.1625 | 0.325 | 0.65 | 1.3 |
| BHA | 0.01 | 0.01 | 0.01 | 0.01 | 0.01 |
| BHT | 0.005 | 0.005 | 0.005 | 0.005 | 0.005 |
| L-Menthol | 0.05 | 0.05 | 0.05 | 0.05 | 0.05 |
| Ethanol | 55 | 55 | 55 | 55 | 55 |
| Propylene Glycol | 5 | 5 | 5 | 5 | 5 |
| Purified Water | 39.8537 | 39.7725 | 39.61 | 39.285 | 38.635 |
| Citric Acid Anhydrous | QS to pH | QS to pH | QS to pH | QS topH | QS to pH |
| Sodium Hydroxide | QS to pH | QS to pH | QS to pH | QS to pH | QS to pH |
| Nitrogen | Sparging/Overlay | Sparging/Overlay | Sparging/Overlay | Sparging/Overlay | Sparging/Overlay |

Values = % w/w.

Formulations #15, #16 and #17 are used in the clinical trial listed as Example 8 for acute pain indication, whereas formulations #14, #15, #16, #17 and #18 will be used in chronic pain indication.
Formulations #14, #15, #16, #17 and #18 represent 0.0625 mg, 0.125 mg, 0.25 mg, 0.5 mg and 1 mg doses, respectively. (Equivalent to buprenorphine base).

Buprenorphine formulations of Table 43 were all stable upon preparation.

### Example 7. Further Buprenorphine/Naloxone Formulations

TABLE 44
Further Buprenorphine/Naloxone Formulations

| Formulation | #19 | #20 | #21 | #22 | #23 | #24 | #25 | #26 | #27 |
|---|---|---|---|---|---|---|---|---|---|
| Buprenorphine HCl | 8.39 | 7.68 | 2.84 | 1.42 | 5.70 | 3.75 | 1.04 | 5.167 | 1.302 |
| NaloxoneHCl Dihydrate | 2.37 | 2.19 | 0.80 | 0.40 | 1.61 | 1.06 | 0.29 | 0.878 | 0.221 |
| L-Menthol | 0.05 | 0.05 | 0.05 | 0.05 | 0.05 | 0.05 | 0.05 | 0.05 | 0.05 |
| Edetate Disodium | 0.005 | 0.005 | 0.005 | 0.005 | 0.005 | 0.005 | 0.005 | 0.005 | 0.005 |
| Dihydrate | | | | | | | | | |
| Sodium Ascorbate | 0.02 | 0.02 | 0.02 | 0.02 | 0.02 | 0.02 | 0.02 | 0.02 | 0.02 |
| Ethanol | 55 | 55 | 55 | 55 | 55 | 55 | 55 | 55 | 55 |
| Propylene Glycol | 5 | 5 | 5 | 5 | 5 | 5 | 5 | 5 | 5 |
| Water | 29.165 | 30.059 | 36.281 | 38.103 | 32.614 | 35.114 | 38.596 | 33.880 | 38.402 |

Values = % w/w.

Buprenorphine/naloxone formulations of Table 44 were all stable upon preparation.

### Example 8: Method of Treatment of Pain Using Buprenorphine Specifications of the Study

This was a multicenter, randomized, double-blind, multiple-dose, placebo-controlled study evaluating the efficacy and safety of three dosing regimens of Buprenorphine Sublingual Spray (0.5 mg (formulation #17) three times daily (“tid”), 0.25 mg (formulation #16) tid, or 0.125 mg (formulation #15) tid), and/or matching placebo in subjects with moderate to severe postoperative pain after bunionectomy. 322 subjects were randomized. 298 subjects completed the study, and 24 discontinued for various reasons (9 to lack of efficacy; 14 due to nausea and emesis; and 1 for non-related hypotension); and one lost to follow-up.

The study lasted four months and comprised 4 periods: The Screening Period (Days −28 to −1), the Surgical Period (Day 0), the Treatment Period (48 hours; Days 1 to 3) and the Follow-up Period (Days 5 to 9).

The measurements of pain intensity and pain relied were conducted at Time 0 (i.e., at 5, 15, 30, and 45 minutes, and 1, 1.5, 2, 3, 4, 5, 6, 7, 8, 12, 16, 20, 24, 32, 40, and 48 hours).

As agreed with the U.S. Food and Drug Administration (“FDA”), the primary efficacy endpoint in this study was the Summed Pain Intensity Difference relative to baseline over a period of 48 hours (SPID-48). The patient assessment of pain intensity utilized a numeric pain scale (11-point scale with 0=no pain to 10=worst possible pain).

The secondary variables were as follows:
SPID over 0 to 4 hours (SPID-4), over 0 to 8 hours (SPID-8), and over 0 to 24 hours (SPID-24) after Time 0;
Time to onset of analgesia (measured as time to perceptible pain relief confirmed by meaningful pain relief using the 2-stopwatch method); and
Pain intensity difference (PID) at each scheduled time point after Time 0.

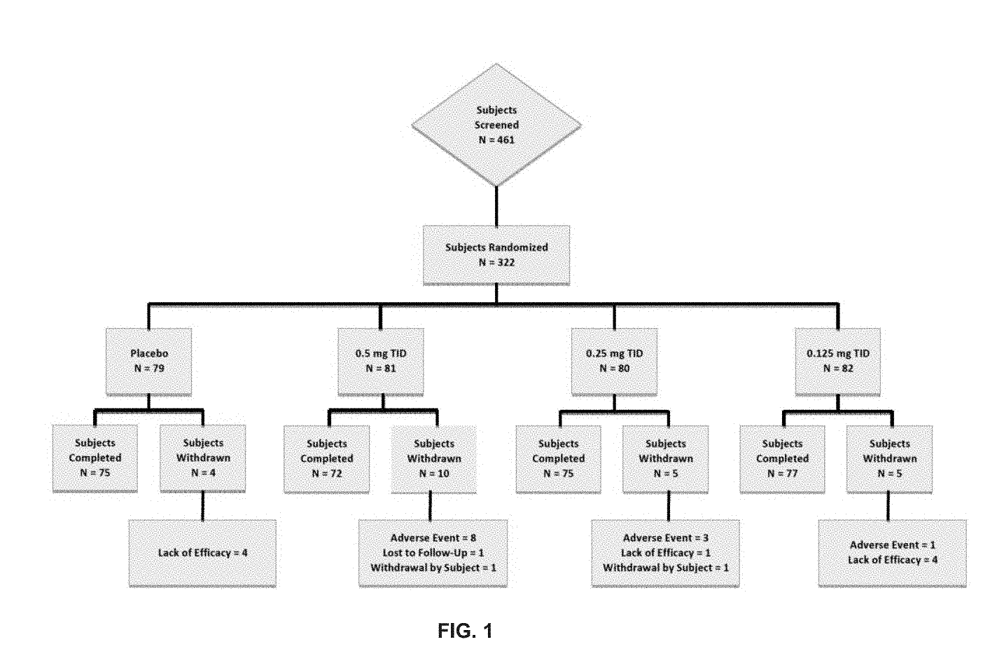

The disposition of subjects is depicted in the flow chart in FIG. 1.

Results

The primary efficacy endpoint was statistically significant at all doses studied. The Buprenorphine Sublingual Spray 0.5 mg tid demonstrated the largest reduction in SPID-48 and was statistically significant to placebo (p<0.0001). The 0.25 mg tid and 0.125 mg tid doses also demonstrated statistically significant reductions in SPID-48 (p=0.0108 and p=0.0120, respectively). All treatments were generally well tolerated.

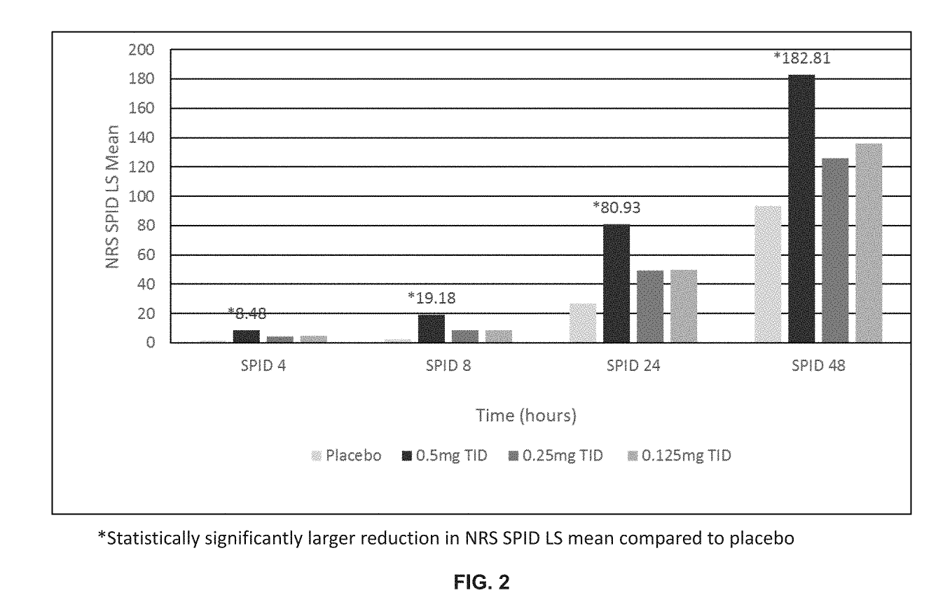

FIG. 2 depicts a chart of Numeric Rating Scale (NRS) Summed Pain Intensity Difference (SPID) at 4, 8, 24 and 48 hours.

Table 45 below describes NRS SPID over 0 to 48 hours (NRS SPID-48) for intention-to-treat (ITT) population.

TABLE 45
Summary of SPID-48
(ITT Population)

| | Buprenorphine Sublingual Spray | | | | | | | |
|---|---|---|---|---|---|---|---|---|
| | Placebo | | 0.5 mg TID | | 0.25 mg TID | | 0.125 mg TID | |
| Statistic | (N = 79) | | (N = 81) | | (N = 80) | | (N = 82) | |
| n | 75 | | 72 | | 75 | | 77 | |
| mean (SD) | 93.40 | (85.063) | 182.81 | (107.349) | 125.75 | (102.247) | 135.84 | (114.040) |
| CV | 91.07 | | 58.72 | | 81.31 | | 83.95 | |
| median | 84.0 | | 181.0 | | 98.0 | | 130.3 | |
| min, max | −77.7, | 377.8 | −17.8, | 414.6 | −55.5, | 399.0 | −90.5, | 399.4 |
| Least square mean(SE)^{a} | 89.40 | (10.109) | 171.33 | (10.316) | 125.58 | (10.101) | 124.85 | (9.944) |
| 95% CI | 69.50, | 109.29 | 151.02, | 191.63 | 105.70, | 145.46 | 105.28, | 144.43 |

| | Least square mean | | |
|---|---|---|---|
| Comparison | difference (SE)^{a} | 95% CI | P-value^{a} |
| 0.5 mg vs. placebo | 81.93 (14.283) | 53.82, 110.04 | <0.0001 |
| 0.25 mg vs. placebo | 36.18 (14.099) | 8.43, 63.93 | 0.0108 |
| 0.125 mg vs. placebo | 35.46 (14.020) | 7.86, 63.05 | 0.0120 |

Note:
SPID-48 = Summary of Pain Intensity Differences over 48 hours,
CV = coefficient of variation,
TID = three times daily.
^{a}Least square means, standard errors(SE), confidence interval(CI) and p-values are from an ANCOVA model with factors for treatment, site and baseline pain intensity.

Table 46 below describes NRS SPID over 0 to 24 hours (NRS SPID-24) for ITT population.

TABLE 46
Summary of SPID-24
(ITT Population)

| | Buprenorphine Sublingual Spray | | | | | | | |
|---|---|---|---|---|---|---|---|---|
| | Placebo | | 0.5 mg TID | | 0.25 mg TID | | 0.125 mg TID | |
| Statistic | (N = 79) | | (N = 81) | | (N = 80) | | (N = 82) | |
| n | 75 | | 73 | | 76 | | 77 | |
| mean (SD) | 26.61 | (42.855) | 80.93 | (53.234) | 49.21 | (48.223) | 49.90 | (56.899) |
| CV | 161.02 | | 65.78 | | 97.98 | | 114.02 | |
| median | 21.0 | | 83.4 | | 43.2 | | 48.3 | |
| min, max | −46.3, | 161.8 | −30.8, | 196.9 | −40.9, | 177.5 | −62.8, | 175.9 |
| Least square mean(SE)^{a} | 24.16 | (5.001) | 75.67 | (5.066) | 48.85 | (4.962) | 44.17 | (4.920) |
| 95% CI | 14.31, | 34.00 | 65.70, | 85.64 | 39.08, | 58.62 | 34.49, | 53.86 |

| | Least square mean | | |
|---|---|---|---|
| Comparison | difference (SE)^{a} | 95% CI | P-value^{a} |
| 0.5 mg vs. placebo | 51.51 (7.041) | 37.66, 65.37 | <0.0001 |
| 0.25 mg vs. placebo | 24.69 (6.952) | 11.01, 38.38 | 0.0004 |
| 0.125 mg vs. placebo | 20.02 (6.937) | 6.37, 33.67 | 0.0042 |

Note:
SPID-24 = Summary of Pain Intensity Differences over 24 hours,
CV = coefficient of variation,
TID = three times daily.
^{a}Least square means, standard errors(SE), confidence interval(CI) and p-values are from an ANCOVA model with factors for treatment, site and baseline pain intensity.

Table 47 below describes NRS SPID over 0 to 8 hours (NRS SPID-8) for ITT population.

TABLE 47
Summary of SPID-8
(ITT Population)

| | Buprenorphine Sublingual Spray | | | | | | | |
|---|---|---|---|---|---|---|---|---|
| | Placebo | | 0.5 mg TID | | 0.25 mg TID | | 0.125 mg TID | |
| Statistic | (N = 79) | | (N = 81) | | (N = 80) | | (N = 82) | |
| n | 77 | | 78 | | 78 | | 78 | |
| mean (SD) | 2.14 | (13.589) | 19.18 | (19.606) | 8.63 | (17.661) | 8.71 | (18.707) |
| CV | 633.82 | | 102.20 | | 204.61 | | 214.72 | |
| median | 0.8 | | 19.2 | | 7.5 | | 6.1 | |
| min, max | −25.1, | 36.3 | −26.7, | 65.3 | −23.3, | 63.1 | −27.8, | 57.2 |
| Least square mean(SE)^{a} | 1.32 | (1.851) | 17.57 | (1.835) | 8.26 | (1.843) | 7.08 | (1.837) |
| 95% CI | −2.32, | 4.97 | 13.96, | 21.18 | 4.63, | 11.89 | 3.47, | 10.70 |

| | Least square mean | | |
|---|---|---|---|
| Comparison | difference (SE)^{a} | 95% CI | P-value^{a} |
| 0.5 mg vs. placebo | 16.24 (2.582) | 11.16, 21.32 | <0.0001 |
| 0.25 mg vs. placebo | 6.93 (2.579) | 1.86, 12.01 | 0.0076 |
| 0.125 mg vs. placebo | 5.76 (2.582) | 0.68, 10.84 | 0.0265 |

Note:
SPID-8 = Summary of Pain Intensity Differences over 8 hours,
CV = coefficient of variation,
TID = three times daily.
^{a}Least square means, standard errors(SE), confidence interval(CI) and p-values are from an ANCOVA model with factors for treatment, site and baseline pain intensity.

Table 48 below describes NRS SPID over 0 to 4 hours (NRS SPID-4) for ITT population.

TABLE 48
Summary of SPID-4
(ITT Population)

| | | Buprenorphine Sublingual Spray | | |
|---|---|---|---|---|
| | Placebo | 0.5 mg TID | 0.25 mg TID | 0.125 mg TID |
| Statistic | (N = 79) | (N = 81) | (N = 80) | (N = 82) |
| n | 78 | 81 | 80 | 80 |
| mean (SD) | 1.29 (8.466) | 8.48 (10.089) | 4.15 (9.230) | 4.59 (10.637) |
| CV | 656.18 | 119.05 | 222.41 | 231.79 |
| median | 0.0 | 8.2 | 4.0 | 2.9 |
| min, max | −20.3, 25.3 | −19.1, 30.2 | −17.2, 27.1 | −22.2, 28.5 |
| Least square | 0.67 (1.036) | 7.70 (1.013) | 3.67 (1.023) | 3.74 (1.020) |
| mean(SE)^{a} | | | | |
| 95% CI | −1.37, 2.70 | 5.71, 9.69 | 1.66, 5.68 | 1.73, 5.75 |
| | Least square | | | |
| | mean difference | | | |
| Comparison | (SE)^{a} | | 95% CI | P-value^{a} |
| 0.5 mg vs. | 7.03 (1.436) | | 4.21, 9.86 | <0.0001 |
| placebo | | | | |
| 0.25 mg vs. | 3.00 (1.439) | | 0.17, 5.84 | 0.0377 |
| placebo | | | | |
| 0.125 mg vs. | 3.07 (1.441) | | 0.24, 5.91 | 0.0337 |
| placebo | | | | |

Note:
SPID-4 = Summary of Pain Intensity Differences over 4 hours, CV = coefficient of variation, TID = three times daily.
^{a}Least square means, standard errors(SE), confidence interval(CI) and p-values are from an ANCOVA model with factors for treatment, site and baseline pain intensity.

Table 49 shows time of onset analgesia for investigator initiated trials (IIT) population.

TABLE 49
Time to Onset of Analgesia
(ITT Population)

| | | | Buprenorphine Sublingual Spray | | | | | |
|---|---|---|---|---|---|---|---|---|
| | Placebo | | 0.5 mg TID | | 0.25 mg TID | | 0.125 mg TID | |
| | (N = 79) | | (N = 81) | | (N = 80) | | (N = 82) | |
| Number (%) of subjects | 27 | (34.2) | 53 | (65.4) | 37 | (46.3) | 36 | (43.9) |
| with onset of analgesia | | | | | | | | |
| Number (%) of subjects | 52 | (65.8) | 28 | (34.6) | 43 | (53.8) | 46 | (56.1) |
| censored | | | | | | | | |
| Time (minutes) from first | | | | | | | | |
| dose to onset of analgesia^{a} | | | | | | | | |
| 25^{th} percentile (95% CI) | 5.0 | (4.0, 83.0) | 6.0 | (5.0, 15.0) | 13.0 | (5.0, 29.0) | 15.0 | (6.0, 27.0) |
| Median (95% CI) | NE | | 43.0 | (21.0, 64.0) | NE | (43.0, NE) | NE | (41.0, NE) |
| 75^{th} percentile (95% CI) | NE | | NE | (101.0, NE) | NE | | NE | |
| Mean (SE) | 58.4 | (4.11) | 146.4 | (21.35) | 58.7 | (4.46) | 58.3 | (4.30) |

| Comparison | P-value^{b} |
|---|---|
| 0.5 mg vs. placebo | 0.0010 |
| 0.25 mg vs. placebo | 0.3018 |
| 0.125 mg vs. placebo | 0.3701 |

^{a}Percentile estimates and confidence intervals (CI) are from a Kaplan-Meier analysis.
^{b}P-value from a log-rank test of each treatment arm vs. placebo
Note:
TID = three times daily, NE = not estimable. Denominator for percentages is the number of subjects per treatment group in the ITT population.
Time to onset of analgesia is the time when the first stopwatch is stopped given that the second stopwatch is stopped.
If the second stopwatch is not stopped, time will be censored at the time of the second dose of study drug or the use of rescue medication, whichever comes first.
If both stopwatches are not stopped, time will be censored at the time of the second dose of study drug or the use of rescue medication whichever comes first.

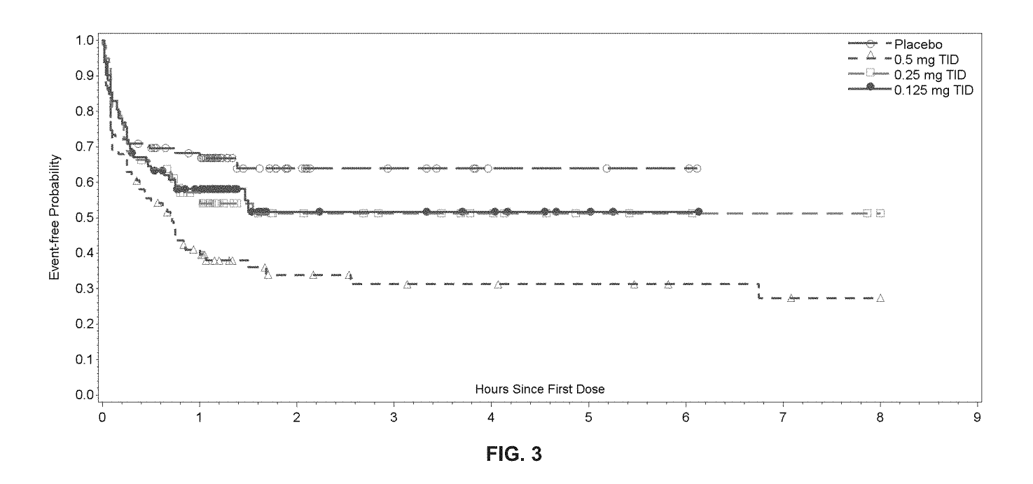

FIG. 3 depicts a chart of time of onset of analgesia for placebo, 0.5 mg tid, 0.25 mg tid and 0.125 tid doses.

Table 50 is a representation of mean pain intensity differences by timepoint.

TABLE 50

| | | | Placebo | 0.5 mg TID | 0.25 mg TID | 0.125 mg TID |
|---|---|---|---|---|---|---|
| Timepoint | | Statistic | (N = 79) | (N = 81) | (N = 80) | (N = 82) |
| 5 | minutes | n | 79 | 81 | 80 | 82 |
| | | mean (SD) | 0.3 (1.06) | 0.5 (1.15) | 0.3 (1.01) | 0.3 (0.75) |
| 15 | minutes | n | 79 | 81 | 80 | 82 |
| | | mean (SD) | 0.6 (1.76) | 0.4 (1.39) | 0.6 (1.56) | 0.6 (1.55) |
| 30 | minutes | n | 79 | 81 | 80 | 82 |
| | | mean (SD) | 0.7 (2.15) | 0.6 (1.65) | 0.7 (2.12) | 0.7 (2.18) |
| 45 | minutes | n | 79 | 81 | 80 | 81 |
| | | mean (SD) | 0.6 (2.38) | 1.1 (2.08) | 0.9 (2.13) | 1.0 (2.41) |
| 1 | hour | n | 79 | 81 | 80 | 81 |
| | | mean (SD) | 0.6 (2.58) | 1.5 (2.40) | 1.0 (2.37) | 1.1 (2.62) |
| 1.5 | hours | n | 78 | 81 | 80 | 80 |
| | | mean (SD) | 0.6 (2.77) | 2.1 (2.72) | 1.2 (2.58) | 1.2 (2.91) |
| 2 | hours | n | 78 | 81 | 79 | 80 |
| | | mean (SD) | 0.5 (2.77) | 2.4 (3.07) | 1.2 (2.62) | 1.3 (3.05) |
| 3 | hours | n | 78 | 81 | 80 | 80 |
| | | mean (SD) | 0.2 (2.35) | 2.7 (3.09) | 1.3 (2.95) | 1.3 (3.22) |
| 4 | hours | n | 78 | 81 | 80 | 80 |
| | | mean (SD) | −0.1 (2.13) | 2.6 (3.26) | 0.9 (3.14) | 1.1 (3.21) |
| 5 | hours | n | 77 | 80 | 79 | 80 |
| | | mean (SD) | −0.4 (2.08) | 2.6 (3.06) | 0.8 (3.14) | 1.2 (3.32) |
| 6 | hours | n | 78 | 78 | 79 | 79 |
| | | mean (SD) | 0.2 (2.16) | 3.1 (3.16) | 1.1 (3.16) | 1.1 (3.19) |
| 7 | hours | n | 77 | 79 | 79 | 78 |
| | | mean (SD) | 0.4 (2.11) | 3.0 (3.06) | 1.3 (2.88) | 0.9 (2.93) |
| 8 | hours | n | 77 | 78 | 78 | 78 |
| | | mean (SD) | 0.5 (2.10) | 2.5 (3.06) | 1.2 (2.78) | 0.7 (2.73) |
| 12 | hours | n | 75 | 78 | 77 | 78 |
| | | mean (SD) | 1.0 (2.50) | 3.7 (2.78) | 2.2 (2.88) | 2.0 (3.40) |
| 16 | hours | n | 75 | 76 | 76 | 77 |
| | | mean (SD) | 0.9 (2.11) | 3.4 (2.65) | 1.9 (2.63) | 1.9 (3.16) |
| 20 | hours | n | 75 | 75 | 76 | 77 |
| | | mean (SD) | 2.1 (2.90) | 4.3 (2.70) | 3.2 (2.69) | 3.1 (3.07) |
| 24 | hours | n | 75 | 73 | 76 | 77 |
| | | mean (SD) | 2.2 (2.59) | 4.0 (2.64) | 3.0 (2.72) | 3.2 (2.98) |
| 32 | hours | n | 75 | 72 | 75 | 77 |
| | | mean (SD) | 2.4 (2.48) | 3.9 (2.89) | 2.9 (2.80) | 3.4 (2.90) |
| 40 | hours | n | 75 | 71 | 75 | 77 |
| | | mean (SD) | 2.5 (2.21) | 3.9 (2.81) | 3.2 (2.58) | 3.3 (2.80) |
| 48 | hours | n | 75 | 72 | 75 | 77 |
| | | mean (SD) | 3.5 (2.60) | 4.9 (2.33) | 3.5 (3.00) | 4.1 (2.89) |

The conclusions are as follows:

### Primary Efficacy

The largest pain reduction (NRS SPID-48) was observed for the 0.5 mg TID BSS group.

Statistically significantly larger reductions in NRS SPID-48 compared to placebo for the 0.5 mg TID BSS p-value: <0.0001. The largest reduction in NRS SPID-48 compared to placebo was observed for the 0.5 mg TID BSS treatment group.

### Secondary Efficacy

Largest pain reductions (NRS SPID-4, NRS SPID-8, and NRS SPID-24) were observed for 0.5 mg TID BSS group (p-value: <0.0001). Secondary time points at 4, 8 and 24 hours SPID were all statistically significantly different.

### Example 9: Pharmacokinetic Data for Formulation 20

Objective

The primary objective of this study was to compare the bioavailability of a test formulation of Buprenorphine-Naloxone Sublingual (SL) spray, 6.5 mg/1.63 mg (1 spray) to that of a single dose of Suboxone® (buprenorphine and naloxone) sublingual film, 12 mg/3 mg, under fasted conditions. The secondary objective was to evaluate the safety and tolerability of Buprenorphine-Naloxone SL spray.

Study Design

This was a single-dose, open-label, randomized, two-period, two-treatment crossover study. Fifty-six healthy subjects were enrolled. Subjects who successfully completed the screening process checked into the research center the evening before first dose. Subjects who continued to meet inclusion/exclusion criteria the morning of dose were assigned a subject number, based on the order in which they successfully completed the screening process and procedures as outlined in the study protocol. Subjects were randomly assigned to a treatment sequence and received two separate single-dose administrations of study medication, one treatment per period, according to the randomization schedule. Dosing days were separated by a washout period of at least 14 days.

Subjects received each of the treatments listed below during the two treatment periods:

Treatment A: Test Product

Buprenorphine Naloxone SL spray, 6.5 mg/1.63 mg

Dose=1 sublingual spray (total dose 6.5 mg/1.63 mg)

Treatment B: Reference Product

Suboxone® (buprenorphine and naloxone) sublingual film, 12 mg/3 mg

Dose=1×12 mg/3 mg sublingual film

Clinical Procedures Summary

During each study period, 6 mL blood samples were obtained for buprenorphine, norbuprenorphine, and unconjugated naloxone analysis before and after each dose at selected times through 144 hours after dose administration. A total of 34 pharmacokinetic (PK) blood samples were collected from each subject for buprenorphine, norbuprenorphine, and unconjugated naloxone, 17 samples in each study period. In addition, 6 mL blood samples were obtained for total naloxone analysis before and after each dose at selected times through 72 hours after dose administration. A total of 28 PK blood samples were collected from each subject for naloxone analysis, 14 samples in each study period.

### Procedures for Collecting Samples for Pharmacokinetic Analysis

Blood samples (1×6 mL) for buprenorphine, norbuprenorphine, and unconjugated naloxone analysis were collected at 0 (predose), and at 5 minutes, 10 minutes, 15 minutes, 30 minutes, and 1, 2, 4, 8, 12, 24, 36, 48, 72, 96, 120, and 144 hours.

Blood samples (1×6 mL) for total naloxone analysis were collected at 0 (predose), and at 5 minutes, 10 minutes, 15 minutes, 30 minutes, and 1, 2, 4, 8, 12, 24, 36, 48, and 72 hours.

### Bioanalytical Summary

Plasma samples were analyzed for buprenorphine, norbuprenorphine, unconjugated naloxone, and total naloxone by Worldwide Clinical Trials (WCT) using validated LC-MS-MS procedures. The methods were validated for ranges of 20.0 to 10,000 pg/mL for buprenorphine and norbuprenorphine and 2.00 to 1000 pg/mL for unconjugated naloxone, based on the analysis of 1.00 mL of human EDTA plasma, and 0.0500 to 50.0 ng/mL for total naloxone, based on the analysis of 0.200 mL of human EDTA plasma. Data were stored in Watson Laboratory Information Management System (LIMS; Version 7.2.0.03, Thermo Fisher Scientific).

### Pharmacokinetic Analysis

Concentration-time data were analyzed using noncompartmental methods in Phoenix™ WinNonlin® (Version 6.3, Pharsight Corporation). Concentration-time data that were below the limit of quantification (BLQ) were treated as zero in the data summarization and descriptive statistics. In the pharmacokinetic analysis, BLQ concentrations were treated as zero from time-zero up to the time at which the first quantifiable concentration was observed; embedded and/or terminal BLQ concentrations were treated as “missing”. Actual sample times were used in the pharmacokinetic analysis. The linear trapezoidal method was used to calculation the area under the curve (AUC).

The following pharmacokinetic parameters were calculated: peak concentration in plasma (Cₘₐₓ), time to peak concentration (Tₘₐₓ), elimination rate constant (λ_{z}), terminal half-life (T_{1/2}), area under the concentration-time curve from time-zero to the time of the last quantifiable concentration (AUCₗₐₛₜ), area under the plasma concentration time curve from time-zero extrapolated to infinity (AUC_{inf}), the percent of AUC_{inf} based on extrapolation (AUCₑₓₜᵣₐₚ), last quantifiable plasma concentration (Cₗₐₛₜ), and time of the last quantifiable plasma concentration (Tₗₐₛₜ). In addition, partial AUCs AUC₀₋₇₂, AUC₀₋₉₆, AUC₀₋₁₂₀, and AUC₀₋₁₄₄ were estimated for buprenorphine and unconjugated naloxone to provide information regarding systemic exposure at different times during the extended pharmacokinetic sampling interval.

Analysis of variance (ANOVA) and the Schuirmann's two one-sided t-test procedure at the 5% significance level were applied to the log-transformed pharmacokinetic exposure parameters, Cₘₐₓ, AUCₗₐₛₜ, and AUC_{inf} for buprenorphine, norbuprenorphine, unconjugated naloxone, and total naloxone. The ratio of the geometric means (Insys Sublingual Spray-Test/Suboxone Sublingual Film-Reference) was reported along with the 90% confidence interval about the ratio. For informational purposes, AUC₀₋₇₂, AUC₀₋₉₆, AUC₀₋₁₂₀, and AUC₀₋₁₄₄ for buprenorphine and unconjugated naloxone were compared across treatments using an analogous statistical method.

### Results and Discussion

Data from 50 subjects who completed at least one study period were included in the pharmacokinetic and statistical analyses. Mean concentration-time data are shown in Tables 51 through 54. Results of the pharmacokinetic and statistical analyses are shown below in Tables 55 through 64.

Buprenorphine

Overall, the pharmacokinetic profile of buprenorphine after the administration of Buprenorphine Naloxone SL spray, 6.5 mg/1.63 mg was similar to that after the administration of Suboxone Sublingual Film 12 mg/3 mg. From the mean buprenorphine concentration-time profiles, the concentrations achieved after the Sublingual Spray were comparable to those after Suboxone, even though a much lower Sublingual Spray dose was administered (6.5 mg in Sublingual Spray vs. 12 mg in Suboxone). At early time points and through approximately 24 hours, the mean buprenorphine concentration-time profiles were practically superimposable for the two treatments; at latter time points, minor differences were noted, with the mean buprenorphine concentrations after the Sublingual Spray being slightly lower than those after Suboxone. These trends were reflected in the derived pharmacokinetic parameters. No appreciable differences were noted in the mean buprenorphine Cₘₐₓ across treatments (5670±1590 pg/mL after Sublingual Spray, 6210±3110 pg/mL after Suboxone). No appreciable differences were noted in the mean±SD buprenorphine AUC₀₋₇₂, AUC₀₋₉₆, AUC₀₋₁₄₄, AUCₗₐₛₜ, and AUC_{inf}. For example, mean AUCₗₐₛₜ was 46660±12980 h*pg/mL after Sublingual Spray and 56100±21460 h*pg/mL after Suboxone. Due to the extended pharmacokinetic sampling interval used in this study, AUC to the last quantifiable sample (AUCₗₐₛₜ) provided a reasonable estimate of the overall systemic exposure (AUC_{inf}, extrapolated to infinity). Mean AUCs values were 48790±13810 h*pg/mL after Sublingual Spray and 59240±22500 h*pg/mL after Suboxone. On average, only 4.27 to 5.28% of AUC_{inf} was based on extrapolation.

It should be noted that some degree of pharmacokinetic variability was observed, in particular for Suboxone relative to that for the Sublingual Spray; the intersubject variability (CV %) for Cₘₐₓ and AUCs ranged from 27.81 to 28.31% for the Sublingual Spray and 37.98 to 50.02% for Suboxone. It was also noted that a differential location shift existed between the mean and median AUC values for Suboxone; the mean AUCₗₐₛₜ and AUC_{inf} values for Suboxone were higher than the median, suggesting that the data were skewed toward the upper range. The differential distribution of the AUC_{inf} values between the two treatments may have contributed to the ANOVA results for this metric (discussed below).

From the statistical analysis log-transformed pharmacokinetic parameters using an ANOVA model, the geometric mean ratios (90% confidence interval) for buprenorphine Cₘₐₓ, AUCₗₐₛₜ, and AUC_{inf} were 96.01% (88.29, 104.42%), 86.11% (80.44, 92.18%), and 85.19% (79.64, 91.12%), respectively. The ANOVA results for buprenorphine AUC₀₋₇₂, AUC₀₋₉₆, AUC₀₋₁₂₀, and AUC₀₋₁₄₄ were 88.40% (82.59, 94.62%), 87.37% (81.68, 93.46%), 86.75% (81.12, 92.77%), and 86.31% (80.72, 92.29%), respectively. Hence, based on actual data over the 144-hour sampling interval (and over truncated intervals through 72, 96, 120 and 144 hours), bioequivalence criteria were met for buprenorphine in comparisons of the Sublingual Spray to Suboxone. The lower 90% confidence interval for the extrapolated AUC (AUC_{inf}) was 79.64%, 0.36% below the standard bioequivalence limit (80.00%) using the two one-sided tests procedure.

Norbuprenorphine

Exposure to norbuprenorphine differed across treatments. Based on mean estimates of Cₘₐₓ and AUCs, exposure to norbuprenorphine was 2- to 2.6-fold lower after the Sublingual Spray relative to Suboxone, possibly due to increased direct absorption into systemic circulation and lower presystemic, first-pass metabolism for the Sublingual Spray.

Unconjugated Naloxone

Overall, the pharmacokinetic profile of unconjugated naloxone after the administration of Buprenorphine Naloxone SL spray, 6.5 mg/1.63 mg was similar to that after the administration of Suboxone Sublingual Film 12 mg/3 mg. Based on mean estimates of Cₘₐₓ and AUCs, exposure to unconjugated naloxone was comparable across treatments. Mean Cₘₐₓ was 379±211 pg/mL after Sublingual Spray and 356±149 pg/mL after Suboxone; mean AUCₗₐₛₜ was 887.6±445.4 h*pg/mL after Sublingual Spray and 942.0±430.1 h*pg/mL after Suboxone. AUC_{inf} were similar to AUCₗₐₛₜ values; due to the relatively short T_{1/2} of unconjugated naloxone (approximately 3 to 4 hours), only 2.18 to 2.41% of AUC_{inf} was based on extrapolation.

From the statistical analysis log-transformed pharmacokinetic parameters using an ANOVA model, the geometric mean ratios (90% confidence interval) for unconjugated naloxone Cₘₐₓ, AUCₗₐₛₜ, and AUC_{inf} were 103.72% (93.78, 114.71%), 94.95% (86.93, 103.72%), and 94.69% (86.79, 103.31%), respectively. The ANOVA results for unconjugated naloxone AUC₀₋₇₂, AUC₀₋₉₆, AUC₀₋₁₂₀, and AUC₀₋₁₄₄ were comparable to those for AUCₗₐₛₜ and AUC_{inf}. Hence, bioequivalence criteria were met for all pharmacokinetic metrics considered in the analysis.

Total Naloxone

Exposure to total naloxone differed across treatments. Based on mean estimates of Cₘₐₓ and AUCs, exposure to total naloxone was approximately 2-fold lower after the Sublingual Spray relative to Suboxone, possibly due to increased direct absorption into systemic circulation and lower presystemic, first-pass metabolism/glucuronidation for the Sublingual Spray.

Conclusions

Overall, the pharmacokinetic profile of buprenorphine after the administration of Buprenorphine Naloxone SL spray, 6.5 mg/1.63 mg was similar to that after the administration of Suboxone Sublingual Film 12 mg/3 mg. No significant differences in Cₘₐₓ and AUCs over the 144-hour pharmacokinetic sampling period were observed and bioequivalence criteria (90% confidence intervals within 80.00-125.00%) were met for the AUC at 72 hours (82.6%-94.6%), 96 hours (81.7%-93.5%), 120 hours (81.1%-92.8%), and 144 hours (80.7%-92.3%) postdose. The lower 90% confidence interval for the extrapolated AUC (AUC_{inf}) was 79.64%, 0.36% below the bioequivalence limit of 80.00%. Therefore, based on data acquired over an extended sampling period (144 hours or 6 days), Buprenorphine Naloxone SL spray, 6.5 mg/1.63 mg is considered essentially bioequivalent to Sublingual Film 12 mg/3 mg.

The pharmacokinetic profile of unconjugated naloxone after the administration of Buprenorphine Naloxone SL spray, 6.5 mg/1.63 mg was similar to that after the administration of Suboxone Sublingual Film 12 mg/3 mg. No significant differences in Cₘₐₓ and AUCs were observed and bioequivalence criteria (90% confidence intervals within 80.00-125.00%) were met for all pharmacokinetic metrics considered in the analysis.

TABLE 51
Buprenorphine Concentration-Time Data after Administration of the
Test Product (Treatment A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (pg/mL) | (pg/mL) | (%) | n | (pg/mL) | (pg/mL) | (%) |
| 0.00 | 50 | 0.00 | 0.00 | NC | 49 | 0.00 | 0.00 | NC |
| 0.08 | 49 | 36.9 | 66.2 | 179.77 | 49 | 1.30 | 9.13 | 700.00 |
| 0.17 | 50 | 491 | 463 | 94.30 | 49 | 50.2 | 73.9 | 147.10 |
| 0.25 | 50 | 1220 | 902 | 74.02 | 49 | 254 | 255 | 100.57 |
| 0.50 | 50 | 3270 | 1570 | 48.13 | 49 | 2300 | 1690 | 73.45 |
| 1.00 | 50 | 4990 | 1690 | 33.93 | 49 | 5130 | 3060 | 59.61 |
| 2.00 | 50 | 5420 | 1530 | 28.14 | 49 | 5440 | 2300 | 42.27 |
| 4.00 | 50 | 3580 | 1270 | 35.49 | 49 | 3660 | 2080 | 56.97 |
| 8.00 | 50 | 1150 | 407 | 35.36 | 49 | 1360 | 983 | 72.21 |
| 12.00 | 50 | 576 | 181 | 31.50 | 49 | 798 | 423 | 53.06 |
| 24.00 | 50 | 293 | 90.6 | 30.94 | 49 | 448 | 166 | 37.07 |
| 36.00 | 50 | 212 | 73.7 | 34.76 | 49 | 329 | 121 | 36.70 |
| 48.00 | 50 | 144 | 53.2 | 36.84 | 49 | 218 | 86.4 | 39.56 |
| 72.00 | 50 | 88.8 | 37.2 | 41.95 | 49 | 133 | 54.2 | 40.70 |
| 96.00 | 50 | 59.9 | 28.1 | 46.83 | 49 | 87.6 | 40.6 | 46.40 |
| 120.00 | 50 | 37.6 | 25.0 | 66.57 | 49 | 59.2 | 31.1 | 52.45 |
| 144.00 | 50 | 25.5 | 21.7 | 85.24 | 48 | 42.1 | 30.9 | 73.38 |

Note:
Plasma samples analyzed using a bioanalytical method with a validated range 20.0 to 10,000 pg/mL; concentrations reported in pg/mL to 3 significant figures; concentrations below limit of quantification set to zero (0.00 pg/mL) in the data summarization
NC = Not calculated

TABLE 52
Norbuprenorphine Concentration-Time Data after Administration of the
Test Product (Treatment A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (pg/mL) | (pg/mL) | (%) | n | (pg/mL) | (pg/mL) | (%) |
| 0.00 | 50 | 0.418 | 2.96 | 707.11 | 49 | 0.567 | 3.97 | 700.00 |
| 0.08 | 49 | 1.04 | 5.09 | 489.80 | 49 | 0.651 | 4.56 | 700.00 |
| 0.17 | 50 | 30.4 | 60.1 | 197.69 | 49 | 3.59 | 12.7 | 353.54 |
| 0.25 | 50 | 137 | 203 | 147.89 | 49 | 41.1 | 89.5 | 217.66 |
| 0.50 | 50 | 456 | 421 | 92.37 | 49 | 800 | 1000 | 125.44 |
| 1.00 | 50 | 684 | 478 | 69.85 | 49 | 1990 | 1650 | 82.59 |
| 2.00 | 50 | 740 | 415 | 56.01 | 49 | 1800 | 1080 | 60.10 |
| 4.00 | 50 | 614 | 304 | 49.60 | 49 | 1260 | 674 | 53.60 |
| 8.00 | 50 | 522 | 235 | 45.03 | 49 | 1020 | 535 | 52.18 |
| 12.00 | 50 | 470 | 217 | 46.21 | 49 | 945 | 469 | 49.62 |
| 24.00 | 50 | 466 | 199 | 42.64 | 49 | 984 | 482 | 49.04 |
| 36.00 | 50 | 390 | 150 | 38.38 | 49 | 828 | 384 | 46.44 |
| 48.00 | 50 | 304 | 132 | 43.32 | 49 | 633 | 302 | 47.76 |
| 72.00 | 50 | 212 | 107 | 50.44 | 49 | 435 | 234 | 53.80 |
| 96.00 | 50 | 151 | 87.5 | 57.93 | 49 | 302 | 172 | 56.88 |
| 120.00 | 50 | 108 | 74.4 | 68.68 | 49 | 213 | 141 | 65.91 |
| 144.00 | 50 | 86.3 | 67.1 | 77.79 | 48 | 171 | 132 | 77.70 |

Note:
Plasma samples analyzed using a bioanalytical method with a validated range 20.0 to 10,000 pg/mL; concentrations reported in pg/mL to 3 significant figures; concentrations below limit of quantification set to zero (0.00 pg/mL) in the data summarization

TABLE 53
Unconjugated Naloxone Concentration-Time Data after Administration of
the Test Product (Treatment A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (pg/mL) | (pg/mL) | (%) | n | (pg/mL) | (pg/mL) | (%) |
| 0.00 | 50 | 0.00 | 0.00 | NC | 49 | 0.00 | 0.00 | NC |
| 0.08 | 49 | 50.4 | 51.9 | 103.09 | 49 | 5.08 | 17.7 | 349.10 |
| 0.17 | 50 | 205 | 171 | 83.54 | 49 | 47.5 | 75.0 | 157.82 |
| 0.25 | 50 | 292 | 232 | 79.37 | 49 | 105 | 99.0 | 94.56 |
| 0.50 | 50 | 349 | 199 | 57.08 | 49 | 294 | 164 | 55.74 |
| 1.00 | 50 | 293 | 140 | 47.98 | 49 | 304 | 120 | 39.42 |
| 2.00 | 50 | 166 | 84.8 | 50.93 | 49 | 177 | 86.5 | 48.78 |
| 4.00 | 50 | 54.3 | 30.3 | 55.76 | 49 | 66.8 | 64.8 | 96.90 |
| 8.00 | 50 | 9.06 | 4.90 | 54.14 | 49 | 14.3 | 15.7 | 109.92 |
| 12.00 | 50 | 3.67 | 3.56 | 97.16 | 49 | 6.80 | 6.47 | 95.25 |
| 24.00 | 50 | 0.705 | 1.87 | 265.19 | 49 | 2.14 | 3.18 | 148.44 |
| 36.00 | 50 | 0.00 | 0.00 | NC | 49 | 0.219 | 0.749 | 341.25 |
| 48.00 | 50 | 0.00 | 0.00 | NC | 49 | 0.00 | 0.00 | NC |
| 72.00 | 50 | 0.00 | 0.00 | NC | 49 | 0.00 | 0.00 | NC |
| 96.00 | 50 | 0.00 | 0.00 | NC | 49 | 0.00 | 0.00 | NC |
| 120.00 | 49 | 0.00 | 0.00 | NC | 49 | 0.00 | 0.00 | NC |
| 144.00 | 50 | 0.00 | 0.00 | NC | 48 | 0.00 | 0.00 | NC |

Note:
Plasma samples analyzed using a bioanalytical method with a validated range 2.00 to 1000 pg/mL; concentrations reported in pg/mL to 3 significant figures; concentrations below limit of quantification set to zero (0.00 pg/mL) in the data summarization
NC = Not calculated

TABLE 54
Total Naloxone Concentration-Time Data after Administration of the
Test Product (Treatment A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (ng/mL) | (ng/mL) | (%) | n | (ng/mL) | (ng/mL) | (%) |
| 0.00 | 50 | 0.00 | 0.00 | NC | 49 | 0.00 | 0.00 | NC |
| 0.08 | 49 | 0.150 | 0.275 | 183.48 | 49 | 0.0285 | 0.0991 | 347.54 |
| 0.17 | 50 | 1.44 | 1.81 | 125.87 | 49 | 0.416 | 0.976 | 234.70 |
| 0.25 | 50 | 4.14 | 4.12 | 99.53 | 49 | 2.50 | 4.94 | 197.40 |
| 0.50 | 50 | 8.90 | 6.50 | 73.10 | 49 | 15.7 | 14.1 | 89.82 |
| 1.00 | 50 | 9.19 | 4.45 | 48.44 | 49 | 21.3 | 10.2 | 48.03 |
| 2.00 | 50 | 5.02 | 2.75 | 54.78 | 49 | 9.76 | 4.53 | 46.43 |
| 4.00 | 50 | 1.50 | 0.960 | 64.13 | 49 | 2.67 | 1.33 | 49.92 |
| 8.00 | 50 | 0.846 | 0.554 | 65.50 | 49 | 1.42 | 1.10 | 77.40 |
| 12.00 | 50 | 0.626 | 0.688 | 109.87 | 49 | 1.05 | 0.514 | 48.83 |
| 24.00 | 50 | 0.211 | 0.121 | 57.26 | 49 | 0.428 | 0.281 | 65.62 |
| 36.00 | 50 | 0.0583 | 0.0680 | 116.78 | 49 | 0.126 | 0.0975 | 77.13 |
| 48.00 | 50 | 0.0101 | 0.0281 | 278.20 | 49 | 0.0520 | 0.0733 | 141.05 |
| 72.00 | 50 | 0.00110 | 0.00778 | 707.11 | 49 | 0.00263 | 0.0129 | 490.72 |

Note:
Plasma samples analyzed using a bioanalytical method with a validated range 0.0500 to 50.0 ng/mL; concentrations reported in ng/mL to 3 significant figures; concentrations below limit of quantification set to zero (0.00 ng/mL) in the data summarization
NC = Not calculated

TABLE 55
Pharmacokinetic Parameters of Buprenorphine.

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 1.63 | 0.50 | 30.77 | 49 | 1.66 | 0.72 | 43.56 |
| Median (Range) | | 2.00 | (0.50-2.00) | | | 2.00 | (0.50-4.00) | |
| Cₘₐₓ (pg/mL) | 50 | 5670 | 1590 | 28.08 | 49 | 6210 | 3110 | 50.02 |
| AUCₗₐₛₜ (h*pg/mL) | 50 | 46660 | 12980 | 27.81 | 49 | 56100 | 21460 | 38.25 |
| AUC_{inf} (h*pg/mL) | 50 | 48790 | 13810 | 28.31 | 49 | 59240 | 22500 | 37.98 |
| AUC₀₋₇₂ (h*pg/mL) | 50 | 43040 | 11670 | 27.11 | 49 | 50560 | 19670 | 38.91 |
| AUC₀₋₉₆ (h*pg/mL) | 50 | 44830 | 12140 | 27.07 | 49 | 53210 | 20390 | 38.32 |
| AUC₀₋₁₂₀ (h*pg/mL) | 50 | 46030 | 12500 | 27.16 | 49 | 54980 | 20910 | 38.04 |
| AUC₀₋₁₄₄ (h*pg/mL) | 50 | 46860 | 12790 | 27.30 | 49 | 56230 | 21320 | 37.92 |
| AUC_{Extrap} (%) | 50 | 4.27 | 2.13 | 49.83 | 49 | 5.28 | 3.01 | 57.02 |
| λ_{z} (h⁻¹) | 50 | 0.0175 | 0.0043 | 24.51 | 49 | 0.0172 | 0.0041 | 23.74 |
| T_{1/2} (h) | 50 | 41.84 | 10.15 | 24.26 | 49 | 42.72 | 10.38 | 24.30 |
| Tₗₐₛₜ (h) | 50 | 133.44 | 18.24 | 13.67 | 49 | 137.60 | 14.50 | 10.54 |
| Cₗₐₛₜ (pg/mL) | 50 | 33.2 | 15.3 | 46.10 | 49 | 47.2 | 24.8 | 52.46 |

TABLE 56
Pharmacokinetic Parameters of Norbuprenorphine

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 3.49 | 5.54 | 158.73 | 49 | 3.98 | 7.39 | 185.67 |
| Median (Range) | | 2.00 | (0.50-24.00) | | | 1.00 | (0.50-36.00) | |
| Cₘₐₓ (pg/mL) | 50 | 854 | 461 | 53.99 | 49 | 2220 | 1540 | 69.12 |
| AUCₗₐₛₜ (h*pg/mL) | 50 | 37570 | 14560 | 38.75 | 49 | 77800 | 34820 | 44.76 |
| AUC_{inf} (h*pg/mL) | 50 | 46870 | 22370 | 47.72 | 49 | 93460 | 47480 | 50.81 |
| AUC_{Extrap} (%) | 50 | 16.39 | 13.43 | 81.94 | 49 | 14.15 | 10.41 | 73.53 |
| λ_{z} (h⁻¹) | 50 | 0.0159 | 0.0076 | 47.52 | 49 | 0.0159 | 0.0060 | 37.45 |
| T_{1/2} (h) | 50 | 56.50 | 34.49 | 61.05 | 49 | 50.97 | 23.27 | 45.66 |
| Tₗₐₛₜ (h) | 50 | 141.12 | 9.25 | 6.56 | 49 | 143.48 | 3.42 | 2.39 |
| Cₗₐₛₜ (pg/mL) | 50 | 89.3 | 63.8 | 71.43 | 49 | 173 | 132 | 76.44 |

TABLE 57
Pharmacokinetic Parameters of Unconjugated Naloxone

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 0.56 | 0.28 | 50.10 | 49 | 0.84 | 0.56 | 66.81 |
| Median (Range) | | 0.50 | (0.17-1.03) | | | 1.00 | (0.25-4.00) | |
| Cₘₐₓ (pg/mL) | 50 | 379 | 211 | 55.76 | 49 | 356 | 149 | 41.75 |
| AUCₗₐₛₜ (h*pg/mL) | 50 | 887.6 | 445.4 | 50.18 | 49 | 942.0 | 430.1 | 45.66 |
| AUC_{inf} (h*pg/mL) | 50 | 904.9 | 445.9 | 49.27 | 48 | 942.0 | 411.0 | 43.63 |
| AUC₀₋₇₂ (h*pg/mL) | 50 | 903.4 | 446.3 | 49.40 | 48 | 941.1 | 410.6 | 43.63 |
| AUC₀₋₉₆ (h*pg/mL) | 50 | 904.0 | 446.1 | 49.35 | 48 | 941.6 | 410.9 | 43.64 |
| AUC₀₋₁₂₀ (h*pg/mL) | 50 | 904.3 | 446.0 | 49.32 | 48 | 941.7 | 411.0 | 43.64 |
| AUC₀₋₁₄₄ (h*pg/mL) | 50 | 904.5 | 445.9 | 49.30 | 48 | 941.8 | 411.0 | 43.64 |
| AUC_{Extrap} (%) | 50 | 2.18 | 2.79 | 128.30 | 48 | 2.41 | 1.33 | 55.07 |
| λ_{z} (h⁻¹) | 50 | 0.3617 | 0.1584 | 43.79 | 48 | 0.2547 | 0.1450 | 56.93 |
| T_{1/2} (h) | 50 | 3.48 | 5.51 | 158.21 | 48 | 4.15 | 3.07 | 74.06 |
| Tₗₐₛₜ (h) | 50 | 13.44 | 5.58 | 41.55 | 49 | 18.37 | 8.28 | 45.10 |
| Cₗₐₛₜ (pg/mL) | 50 | 4.00 | 1.90 | 47.39 | 49 | 4.51 | 3.29 | 72.93 |

TABLE 58
Pharmacokinetic Parameters of Total Naloxone

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 1.40 | 2.13 | 152.59 | 49 | 1.12 | 1.14 | 101.89 |
| Median (Range) | | 1.00 | (0.25-12.00) | | | 1.00 | (0.50-8.00) | |
| Cₘₐₓ (ng/mL) | 50 | 12.0 | 5.38 | 44.86 | 49 | 24.9 | 11.9 | 47.75 |
| AUCₗₐₛₜ (h*ng/mL) | 50 | 34.12 | 10.51 | 30.80 | 49 | 65.80 | 20.43 | 31.04 |
| AUC_{inf} (h*ng/mL) | 48 | 36.22 | 10.45 | 28.84 | 49 | 66.99 | 20.39 | 30.44 |
| AUC_{Extrap} (%) | 48 | 4.48 | 2.92 | 65.21 | 49 | 2.31 | 2.89 | 124.97 |
| λ_{z} (h⁻¹) | 48 | 0.0911 | 0.0377 | 41.37 | 49 | 0.0923 | 0.0277 | 30.03 |
| T_{1/2} (h) | 48 | 8.71 | 3.31 | 38.00 | 49 | 8.24 | 2.71 | 32.85 |
| Tₗₐₛₜ (h) | 50 | 32.16 | 10.41 | 32.37 | 49 | 41.14 | 10.95 | 26.63 |
| Cₗₐₛₜ (ng/mL)ANO | 50 | 0.122 | 0.0587 | 48.20 | 49 | 0.0987 | 0.0575 | 58.20 |

TABLE 59
Statistical Analysis of the Log-Transformed
Systemic Exposure Parameters of Buprenorphine

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 5431.5970 | 5657.0376 | 96.01 | 88.29 | 104.42 | 0.9961 | 23.69 |
| ln(AUCₗₐₛₜ) | 45456.8371 | 52788.4107 | 86.11 | 80.44 | 92.18 | 0.9998 | 19.14 |
| ln(AUC_{inf}) | 47445.5869 | 55696.4070 | 85.19 | 79.64 | 91.12 | 0.9998 | 18.91 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

TABLE 60
Statistical Analysis of the Log-Transformed Systemic
Exposure Parameters of Norbuprenorphine

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 703.1707 | 1772.8716 | 39.66 | 36.36 | 43.27 | 0.9941 | 24.60 |
| ln(AUCₗₐₛₜ) | 33655.6801 | 68872.0707 | 48.87 | 45.97 | 51.94 | 1.0000 | 17.14 |
| ln(AUC_{inf}) | 40581.0836 | 81427.3060 | 49.84 | 46.26 | 53.70 | 0.9991 | 21.01 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

TABLE 61
Statistical Analysis of the Log-Transformed Systemic
Exposure Parameters of Unconjugated Naloxone

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 339.8783 | 327.6977 | 103.72 | 93.78 | 114.71 | 0.9764 | 28.62 |
| ln(AUCₗₐₛₜ) | 824.7608 | 868.5854 | 94.95 | 86.93 | 103.72 | 0.9931 | 24.96 |
| ln(AUC_{inf}) | 828.9973 | 875.5051 | 94.69 | 86.79 | 103.31 | 0.9940 | 24.64 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

TABLE 62
Statistical Analysis of the Log-Transformed Systemic
Exposure Parameters of Total Naloxone

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 9.5951 | 20.9370 | 45.83 | 39.77 | 52.82 | 0.8295 | 41.12 |
| ln(AUCₗₐₛₜ) | 30.8413 | 60.7760 | 50.75 | 47.16 | 54.60 | 0.9993 | 20.61 |
| ln(AUC_{inf}) | 32.8603 | 62.0280 | 52.98 | 49.53 | 56.67 | 0.9998 | 18.47 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

TABLE 63
Statistical Analysis of the Log-Transformed Partial AUCs of Buprenorphine

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(AUC₀₋₇₂) | 42137.3401 | 47665.3869 | 88.40 | 82.59 | 94.62 | 0.9998 | 19.12 |
| ln(AUC₀₋₉₆) | 43841.7217 | 50179.2267 | 87.37 | 81.68 | 93.46 | 0.9998 | 18.93 |
| ln(AUC₀₋₁₂₀) | 44970.3912 | 51838.9927 | 86.75 | 81.12 | 92.77 | 0.9998 | 18.84 |
| ln(AUC₀₋₁₄₀) | 45740.0908 | 52993.1994 | 86.31 | 80.72 | 92.29 | 0.9998 | 18.82 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

TABLE 64
Statistical Analysis of the Log-Transformed
Partial AUCs of Unconjugated Naloxone

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(AUC₀₋₇₂) | 827.2680 | 874.7880 | 94.57 | 86.67 | 103.18 | 0.9939 | 24.66 |
| ln(AUC₀₋₉₆) | 827.9833 | 875.1413 | 94.61 | 86.71 | 103.23 | 0.9939 | 24.66 |
| ln(AUC₀₋₁₂₀) | 828.3676 | 875.2500 | 94.64 | 86.74 | 103.27 | 0.9939 | 24.65 |
| ln(AUC₀₋₁₄₄) | 828.5919 | 875.2984 | 94.66 | 86.76 | 103.29 | 0.9940 | 24.65 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

### Example 10: Pharmacokinetic Data for Formulation 21

### Objective

The primary objective of this study was to compare the bioavailability of a test formulation of Buprenorphine-Naloxone Sublingual (SL) spray, 2.2 mg/0.55 mg (1 spray) to that of a single dose of Suboxone (buprenorphine and naloxone) sublingual film, 4 mg/1 mg, under fasted conditions. The secondary objective was to evaluate the safety and tolerability of Buprenorphine-Naloxone SL spray.

### Study Design

This was a single-dose, open-label, randomized, two-period, two-treatment crossover study. Fifty-six healthy subjects were enrolled. Subjects who successfully completed the screening process checked into the research center the evening before first dose. Subjects who continued to meet inclusion/exclusion criteria the morning of dose were assigned a subject number, based on the order in which they successfully completed the screening process and procedures as outlined in the study protocol. Subjects were randomly assigned to a treatment sequence and received two separate single-dose administrations of study medication, one treatment per period, according to the randomization schedule. Dosing days were separated by a washout period of at least 14 days.

Subjects received each of the treatments listed below during the two treatment periods:

Treatment A: Test Product

Buprenorphine Naloxone SL spray, 2.2 mg/0.55 mg

Dose=1 sublingual spray (total dose 2.2 mg/0.55 mg)

Treatment B: Reference Product
Suboxone® (buprenorphine and naloxone) sublingual film, 4 mg/1 mg

Dose=1×4 mg/1 mg sublingual film

### Clinical Procedures Summary

During each study period, 6 mL blood samples were obtained for buprenorphine, norbuprenorphine, and unconjugated naloxone analysis before and after each dose at selected times through 168 hours after dose administration. A total of 36 pharmacokinetic (PK) blood samples were collected from each subject for buprenorphine, norbuprenorphine, and unconjugated naloxone, 18 samples in each study period. In addition, 6 mL blood samples were obtained for total naloxone analysis before and after each dose at selected times through 72 hours after dose administration. A total of 28 PK blood samples were collected from each subject for naloxone analysis, 14 samples in each study period.

### Procedures for Collecting Samples for Pharmacokinetic Analysis

Blood samples (1×6 mL) for buprenorphine, norbuprenorphine, and unconjugated naloxone analysis were collected at 0 (predose), and at 5 minutes, 10 minutes, 15 minutes, 30 minutes, and 1, 2, 4, 8, 12, 24, 36, 48, 72, 96, 120, 144, and 168 hours post dose (18 time points).

Blood samples (1×6 mL) for total naloxone analysis were collected in Vacutainer tubes containing K₂-EDTA as a preservative at 0 (predose), and at 5 minutes, 10 minutes, 15 minutes, 30 minutes, and 1, 2, 4, 8, 12, 24, 36, 48, and 72 hours (14 time points).

Bioanalytical Summary

Plasma samples were analyzed for buprenorphine, norbuprenorphine, unconjugated naloxone, and total naloxone by Worldwide Clinical Trials (WCT) using validated LC-MS-MS procedures. The methods were validated for ranges of 20.0 to 10,000 pg/mL for buprenorphine and norbuprenorphine and 2.00 to 1000 pg/mL for unconjugated naloxone, based on the analysis of 1.00 mL of human EDTA plasma, and 0.0500 to 50.0 ng/mL for total naloxone, based on the analysis of 0.200 mL of human EDTA plasma. Data were stored in Watson Laboratory Information Management System (LIMS; Version 7.2.0.03, Thermo Fisher Scientific). Details of the method validation and sample analysis procedure are provided in the Method Validation Report and Bioanalytical Report sections.

### Pharmacokinetic Analysis

Concentration-time data were analyzed using noncompartmental methods in Phoenix™ WinNonlin® (Version 6.3, Pharsight Corporation). Concentration-time data that were below the limit of quantification (BLQ) were treated as zero in the data summarization and descriptive statistics. In the pharmacokinetic analysis, BLQ concentrations were treated as zero.

The following pharmacokinetic parameters were calculated: peak concentration in plasma (Cₘₐₓ), time to peak concentration (Tₘₐₓ), elimination rate constant (λ_{z}), terminal half-life (T_{1/2}), area under the concentration-time curve from time-zero to the time of the last quantifiable concentration (AUCₗₐₛₜ), area under the plasma concentration time curve from time-zero extrapolated to infinity (AUC_{inf}).

Analysis of variance (ANOVA) and the Schuirmann's two one-sided t-test procedure at the 5% significance level were applied to the log-transformed pharmacokinetic exposure parameters, Cₘₐₓ, AUCₗₐₛₜ, and AUC_{inf}. The 90% confidence interval for the ratio of the geometric means (Test/Reference) was calculated. Bioequivalence was declared if the lower and upper confidence intervals of the log-transformed parameters were within 80% to 125%.

### Results

Data from 52 subjects who completed at least one study period were included in the pharmacokinetic analysis. Data from 50 subjects who completed both study periods were included in the statistical analysis. Mean concentration-time data are shown in Tables 65 through 68. Results of the pharmacokinetic and statistical analyses are shown below in Tables 69 through 76.

### Conclusions

Buprenorphine exposure, based on ln(AUCₗₐₛₜ) and ln(AUC_{inf}), was comparable across treatments and the 90% confidence intervals were within the accepted of 80% to 125% limits for demonstrating similar bioavailability between Buprenorphine Naloxone SL spray, 2.2 mg/0.55 mg and Suboxone sublingual film, 4 mg/1 mg. Buprenorphine Cₘₐₓ was approximately 27% higher after the administration of Buprenorphine Naloxone SL spray, 2.2 mg/0.55 mg compared to that after Suboxone sublingual film, 4 mg/1 mg.

Peak and overall systemic exposure to unconjugated naloxone, based on ln(Cₘₐₓ), ln(AUCₗₐₛₜ), and ln(AUC_{inf}), was approximately 31 to 66% higher after the administration of Buprenorphine Naloxone SL spray, 2.2 mg/0.55 mg compared to that after Suboxone sublingual film, 4 mg/1 mg.

TABLE 65
Buprenorphine Concentration-Time Data after Administration of the
Test Product (Treatment A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (pg/mL) | (pg/mL) | (%) | n | (pg/mL) | (pg/mL) | (%) |
| 0.00 | 50 | 0.00 | 0.00 | NC | 52 | 0.00 | 0.00 | NC |
| 0.08 | 50 | 12.9 | 42.7 | 330.40 | 52 | 0.00 | 0.00 | NC |
| 0.17 | 50 | 170 | 172 | 101.40 | 51 | 5.02 | 12.4 | 246.05 |
| 0.25 | 50 | 514 | 453 | 88.26 | 52 | 69.4 | 90.8 | 130.89 |
| 0.50 | 50 | 1360 | 809 | 59.36 | 52 | 655 | 455 | 69.47 |
| 1.00 | 50 | 2140 | 953 | 44.44 | 52 | 1470 | 633 | 43.22 |
| 2.00 | 50 | 2320 | 850 | 36.58 | 52 | 1930 | 730 | 37.89 |
| 4.00 | 50 | 1530 | 546 | 35.60 | 52 | 1310 | 539 | 41.13 |
| 8.00 | 50 | 498 | 196 | 39.33 | 52 | 494 | 191 | 38.56 |
| 12.00 | 50 | 241 | 85.0 | 35.28 | 52 | 281 | 112 | 39.66 |
| 24.00 | 50 | 120 | 45.9 | 38.13 | 52 | 158 | 66.5 | 42.21 |
| 36.00 | 49 | 80.5 | 25.4 | 31.57 | 52 | 107 | 34.6 | 32.36 |
| 48.00 | 49 | 59.8 | 19.5 | 32.53 | 52 | 76.5 | 25.5 | 33.27 |
| 72.00 | 49 | 31.7 | 15.7 | 49.54 | 52 | 45.0 | 18.5 | 41.08 |
| 96.00 | 49 | 18.1 | 15.4 | 85.22 | 52 | 27.1 | 18.1 | 66.82 |
| 120.00 | 49 | 4.48 | 10.7 | 239.47 | 52 | 12.3 | 15.4 | 125.50 |
| 144.00 | 49 | 2.19 | 7.60 | 347.35 | 52 | 4.58 | 10.5 | 229.39 |
| 168.00 | 49 | 0.580 | 4.06 | 700.00 | 52 | 1.45 | 6.10 | 420.91 |

Note:
Plasma samples analyzed using a bioanalytical method with a validated range 20.0 to 10,000 pg/mL; concentrations reported in pg/mL to 3 significant figures; concentrations below limit of quantification set to zero (0.00 pg/mL) in the data summarization
NC = Not calculated

TABLE 66
Norbuprenorphine Concentration-Time Data after Administration of the
Test Product (Treatment A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (pg/mL) | (pg/mL) | (%) | n | (pg/mL) | (pg/mL) | (%) |
| 0.00 | 50 | 0.00 | 0.00 | NC | 52 | 0.00 | 0.00 | NC |
| 0.08 | 50 | 0.00 | 0.00 | NC | 52 | 0.00 | 0.00 | NC |
| 0.17 | 50 | 4.87 | 22.6 | 463.38 | 51 | 0.613 | 4.38 | 714.14 |
| 0.25 | 50 | 33.2 | 70.6 | 212.49 | 52 | 14.4 | 39.1 | 272.22 |
| 0.50 | 50 | 119 | 149 | 124.82 | 52 | 271 | 393 | 145.18 |
| 1.00 | 50 | 193 | 155 | 80.49 | 52 | 432 | 329 | 76.08 |
| 2.00 | 50 | 217 | 117 | 53.83 | 52 | 461 | 252 | 54.53 |
| 4.00 | 50 | 196 | 89.5 | 45.56 | 52 | 364 | 168 | 46.04 |
| 8.00 | 50 | 179 | 92.1 | 51.45 | 52 | 328 | 167 | 50.84 |
| 12.00 | 50 | 164 | 83.1 | 50.69 | 52 | 305 | 159 | 52.06 |
| 24.00 | 50 | 155 | 74.4 | 48.14 | 52 | 294 | 142 | 48.10 |
| 36.00 | 49 | 130 | 56.8 | 43.66 | 52 | 237 | 97.8 | 41.33 |
| 48.00 | 49 | 106 | 44.8 | 42.23 | 52 | 188 | 79.6 | 42.44 |
| 72.00 | 49 | 70.8 | 30.5 | 43.05 | 52 | 127 | 49.7 | 39.26 |
| 96.00 | 49 | 51.5 | 28.4 | 55.22 | 52 | 90.6 | 44.6 | 49.20 |
| 120.00 | 49 | 30.6 | 24.4 | 79.65 | 52 | 58.5 | 29.6 | 50.58 |
| 144.00 | 49 | 21.2 | 22.5 | 106.17 | 52 | 39.2 | 28.7 | 73.24 |
| 168.00 | 49 | 16.2 | 20.8 | 127.83 | 52 | 29.5 | 28.0 | 94.98 |

Note:
Plasma samples analyzed using a bioanalytical method with a validated range 20.0 to 10,000 pg/mL; concentrations reported in pg/mL to 3 significant figures; concentrations below limit of quantification set to zero (0.00 pg/mL) in the data summarization
NC = Not calculated

TABLE 67
Unconjugated Naloxone Concentration-Time Data after Administration of
the Test Product (Treatment A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (pg/mL) | (pg/mL) | (%) | n | (pg/mL) | (pg/mL) | (%) |
| 0.00 | 50 | 0.00 | 0.00 | NC | 52 | 0.00 | 0.00 | NC |
| 0.08 | 50 | 22.6 | 28.7 | 127.14 | 52 | 0.141 | 0.710 | 505.16 |
| 0.17 | 50 | 69.6 | 48.1 | 69.17 | 51 | 8.12 | 11.0 | 135.57 |
| 0.25 | 50 | 115 | 80.1 | 69.56 | 52 | 27.7 | 32.2 | 116.37 |
| 0.50 | 50 | 140 | 71.9 | 51.43 | 52 | 75.1 | 49.2 | 65.49 |
| 1.00 | 50 | 112 | 50.5 | 44.89 | 52 | 82.3 | 37.7 | 45.84 |
| 2.00 | 50 | 65.3 | 34.4 | 52.62 | 52 | 52.4 | 20.4 | 38.88 |
| 4.00 | 50 | 21.8 | 14.7 | 67.22 | 52 | 18.4 | 10.1 | 54.68 |
| 8.00 | 50 | 3.01 | 2.55 | 84.83 | 52 | 4.09 | 3.42 | 83.71 |
| 12.00 | 50 | 0.480 | 1.23 | 257.25 | 52 | 1.46 | 2.43 | 166.08 |
| 24.00 | 50 | 0.0598 | 0.423 | 707.11 | 52 | 0.297 | 0.944 | 318.00 |
| 36.00 | 49 | 0.00 | 0.00 | NC | 52 | 0.00 | 0.00 | NC |
| 48.00 | 49 | 0.00 | 0.00 | NC | 52 | 0.00 | 0.00 | NC |
| 72.00 | 49 | 0.00 | 0.00 | NC | 52 | 0.00 | 0.00 | NC |
| 96.00 | 49 | 0.00 | 0.00 | NC | 52 | 0.00 | 0.00 | NC |
| 120.00 | 49 | 0.00 | 0.00 | NC | 52 | 0.00 | 0.00 | NC |
| 144.00 | 49 | 0.00 | 0.00 | NC | 52 | 0.00 | 0.00 | NC |
| 168.00 | 49 | 0.00 | 0.00 | NC | 52 | 0.00 | 0.00 | NC |

Note:
Plasma samples analyzed using a bioanalytical method with a validated range 2.00 to 1000 pg/mL; concentrations reported in pg/mL to 3 significant figures; concentrations below limit of quantification set to zero (0.00 pg/mL) in the data summarization
NC = Not calculated

TABLE 68
Total Naloxone Concentration-Time Data after
Administration of the Test Product (Treatment
A) and the Reference Product (Treatment B).

| | Treatment A: | | | | Treatment B: | | | |
|---|---|---|---|---|---|---|---|---|
| | Test Product | | | | Reference Product (Suboxone) | | | |
| Time | | Mean | SD | CV | | Mean | SD | CV |
| (h) | n | (ng/mL) | (ng/mL) | (%) | n | (ng/mL) | (ng/mL) | (%) |
| 0.00 | 50 | 0.00 | 0.00 | NC | 52 | 0.00 | 0.00 | NC |
| 0.08 | 50 | 0.0538 | 0.116 | 216.21 | 52 | 0.00453 | 0.0198 | 437.39 |
| 0.17 | 50 | 0.486 | 0.756 | 155.58 | 51 | 0.105 | 0.307 | 292.49 |
| 0.25 | 50 | 1.36 | 1.60 | 117.10 | 52 | 1.02 | 1.54 | 150.08 |
| 0.50 | 50 | 2.69 | 2.32 | 86.33 | 52 | 7.95 | 6.80 | 85.51 |
| 1.00 | 50 | 3.22 | 2.34 | 72.61 | 52 | 6.28 | 3.58 | 57.03 |
| 2.00 | 50 | 1.74 | 0.983 | 56.49 | 52 | 3.50 | 1.71 | 48.75 |
| 4.00 | 50 | 0.658 | 0.468 | 71.21 | 52 | 1.21 | 0.902 | 74.26 |
| 8.00 | 50 | 0.321 | 0.146 | 45.65 | 52 | 0.570 | 0.290 | 50.96 |
| 12.00 | 50 | 0.198 | 0.0980 | 49.49 | 52 | 0.372 | 0.187 | 50.21 |
| 24.00 | 50 | 0.0679 | 0.0496 | 73.01 | 52 | 0.124 | 0.0642 | 51.62 |
| 36.00 | 49 | 0.00129 | 0.00904 | 700.00 | 52 | 0.0116 | 0.0304 | 261.88 |
| 48.00 | 49 | 0.00 | 0.00 | NC | 52 | 0.00 | 0.00 | NC |
| 72.00 | 49 | 0.00 | 0.00 | NC | 52 | 0.00 | 0.00 | NC |

Note:
Plasma samples analyzed using a bioanalytical method with a validated range 0.0500 to 50.0 ng/mL; concentrations reported in ng/mL to 3 significant figures; concentrations below limit of quantification set to zero (0.00 ng/mL) in the data summarization
NC = Not calculated

TABLE 69
Pharmacokinetic Parameters of Buprenorphine

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 1.68 | 0.73 | 43.26 | 52 | 1.98 | 0.72 | 36.38 |
| Cₘₐₓ (pg/mL) | 50 | 2470 | 850 | 34.35 | 52 | 1990 | 703 | 35.43 |
| AUCₗₐₛₜ (h*pg/mL) | 50 | 18010 | 6118 | 33.97 | 52 | 18240 | 5820 | 31.91 |
| AUC_{inf} (h*pg/mL) | 50 | 19320 | 6190 | 32.04 | 52 | 19590 | 6018 | 30.72 |
| AUC_{Extrap} (%) | 50 | 7.39 | 3.84 | 51.88 | 52 | 7.23 | 2.65 | 36.72 |
| λ_{z} (h⁻¹) | 50 | 0.0244 | 0.0130 | 53.20 | 52 | 0.0217 | 0.0078 | 35.71 |
| T_{1/2} (h) | 50 | 33.99 | 14.07 | 41.40 | 52 | 35.59 | 11.28 | 31.69 |
| Tₗₐₛₜ (h) | 50 | 89.28 | 27.87 | 31.22 | 52 | 105.23 | 28.97 | 27.53 |
| Cₗₐₛₜ (pg/mL) | 50 | 28.2 | 11.0 | 38.96 | 52 | 26.4 | 5.98 | 22.65 |

Note:
Full precision data used in pharmacokinetic analysis

TABLE 70
Pharmacokinetic Parameters of Norbuprenorphine

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 5.54 | 8.06 | 145.49 | 52 | 4.00 | 5.62 | 140.60 |
| Cₘₐₓ (pg/mL) | 50 | 265 | 162 | 61.11 | 52 | 566 | 350 | 61.76 |
| AUCₗₐₛₜ (h*pg/mL) | 50 | 12360 | 5387 | 43.57 | 52 | 23270 | 9030 | 38.80 |
| AUC_{inf} (h*pg/mL) | 50 | 15370 | 6778 | 44.09 | 52 | 26980 | 11550 | 42.82 |
| AUC_{Extrap} (%) | 50 | 19.24 | 12.97 | 67.42 | 52 | 12.48 | 11.68 | 93.57 |
| λ_{z} (h⁻¹) | 50 | 0.0165 | 0.0095 | 57.42 | 52 | 0.0168 | 0.0072 | 42.94 |
| T_{1/2} (h) | 50 | 56.34 | 39.94 | 70.89 | 52 | 53.41 | 42.88 | 80.29 |
| Tₗₐₛₜ (h) | 50 | 131.52 | 38.25 | 29.09 | 52 | 152.77 | 23.78 | 15.56 |
| Cₗₐₛₜ (pg/mL) | 50 | 34.0 | 13.8 | 40.44 | 52 | 39.0 | 20.0 | 51.24 |

Note:
Full precision data used in pharmacokinetic analysis

TABLE 71
Pharmacokinetic Parameters of Unconjugated Naloxone

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 0.54 | 0.26 | 47.66 | 52 | 0.95 | 0.45 | 46.82 |
| Cₘₐₓ (pg/mL) | 50 | 153 | 78.4 | 51.37 | 52 | 89.8 | 43.9 | 48.83 |
| AUCₗₐₛₜ (h*pg/mL) | 50 | 310.2 | 156.8 | 50.57 | 52 | 232.6 | 105.0 | 45.16 |
| AUC_{inf} (h*pg/mL) | 50 | 320.6 | 158.3 | 49.37 | 44 | 262.0 | 110.1 | 42.04 |
| AUC_{Extrap} (%) | 50 | 4.09 | 3.98 | 97.30 | 44 | 4.86 | 2.83 | 58.26 |
| λ_{z} (h⁻¹) | 50 | 0.4972 | 0.1191 | 23.95 | 44 | 0.3643 | 0.1447 | 39.72 |
| T_{1/2} (h) | 50 | 1.58 | 1.06 | 67.25 | 44 | 2.60 | 2.28 | 87.94 |
| Tₗₐₛₜ (h) | 50 | 7.92 | 3.38 | 42.67 | 52 | 10.15 | 5.16 | 50.83 |
| Cₗₐₛₜ (pg/mL) | 50 | 5.28 | 4.51 | 85.35 | 52 | 4.16 | 2.52 | 60.67 |

Note:
Full precision data used in pharmacokinetic analysis

TABLE 72
Pharmacokinetic Parameters of Total Naloxone

| | Treatment A: | | | | Treatment B: | | | |
|---|---|---|---|---|---|---|---|---|
| | Test Product | | | | Reference Product (Suboxone) | | | |
| Parameter | n | Mean | SD | CV % | n | Mean | SD | CV % |
| Tₘₐₓ (h) | 50 | 1.17 | 1.26 | 108.00 | 52 | 1.02 | 0.70 | 68.67 |
| Cₘₐₓ (ng/mL) | 50 | 4.26 | 2.52 | 59.05 | 52 | 9.95 | 5.47 | 54.92 |
| AUCₗₐₛₜ (h*ng/mL) | 50 | 10.68 | 3.908 | 36.60 | 52 | 21.34 | 6.554 | 30.72 |
| AUC_{inf} (h*ng/mL) | 49 | 11.87 | 3.903 | 32.89 | 52 | 22.70 | 6.714 | 29.58 |
| AUC_{Extrap} (%) | 49 | 9.54 | 7.78 | 81.57 | 52 | 6.24 | 3.59 | 57.52 |
| λ_{z} (h⁻¹) | 49 | 0.1161 | 0.0579 | 49.87 | 52 | 0.1066 | 0.0372 | 34.84 |
| T_{1/2} (h) | 49 | 7.21 | 3.33 | 46.21 | 52 | 7.35 | 2.81 | 38.28 |
| Tₗₐₛₜ (h) | 50 | 21.04 | 5.87 | 27.91 | 52 | 24.69 | 5.53 | 22.39 |
| Cₗₐₛₜ (ng/mL) | 50 | 0.102 | 0.0428 | 42.00 | 52 | 0.136 | 0.104 | 76.93 |

Note:
Full precision data used in pharmacokinetic analysis

TABLE 73
Statistical Analysis of the Log-Transformed
Systemic Exposure Parameters of Buprenorphine

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 2334.8796 | 1842.7190 | 126.71 | 114.98 | 139.63 | 0.9827 | 29.55 |
| ln(AUCₗₐₛₜ) | 17009.6037 | 17098.2817 | 99.48 | 91.06 | 108.69 | 0.9930 | 26.82 |
| ln(AUC_{inf}) | 18379.2372 | 18433.5928 | 99.71 | 91.61 | 108.52 | 0.9957 | 25.64 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

TABLE 74
Statistical Analysis of the Log-Transformed Systemic
Exposure Parameters of Norbuprenorphine

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 228.7018 | 489.3838 | 46.73 | 43.28 | 50.47 | 0.9988 | 23.19 |
| ln(AUCₗₐₛₜ) | 11116.0926 | 21710.7037 | 51.20 | 47.09 | 55.67 | 0.9963 | 25.34 |
| ln(AUC_{inf}) | 13986.5409 | 24965.8998 | 56.02 | 51.65 | 60.77 | 0.9974 | 24.59 |

^{a}Geometric Mean tor the Test Product (Test) and Reference Product (Ret) based on Least Squares Mean of log-transformed parameter values
^{b}Ratio(%) = Geometric Mean (Test)/Geometric Mean (Ref)
^{c}90% Confidence Interval

TABLE 75
Statistical Analysis of the Log-Transformed Systemic
Exposure Parameters of Unconjugated Naloxone

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 132.4558 | 79.8936 | 165.79 | 146.96 | 187.03 | 0.9200 | 37.10 |
| ln(AUCₗₐₛₜ) | 275.6491 | 210.1213 | 131.19 | 117.86 | 146.02 | 0.9622 | 32.75 |
| ln(AUC_{inf}) | 287.6305 | 218.5298 | 131.62 | 118.45 | 146.26 | 0.9663 | 29.60 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

TABLE 76
Statistical Analysis of the Log-Transformed Systemic Exposure Parameters of Total Naloxone

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 3.4477 | 8.5476 | 40.34 | 34.96 | 46.53 | 0.8245 | 44.57 |
| ln(AUCₗₐₛₜ) | 9.8049 | 20.6392 | 47.51 | 44.24 | 51.01 | 0.9996 | 21.45 |
| ln(AUC_{inf}) | 11.1098 | 22.0499 | 50.39 | 47.23 | 53.75 | 0.9999 | 19.22 |

^{a}Geometric Mean for the Test Product (Test) and Reference Product (Ref) based on Least Squares Mean of log-transformed parameter values
^{b}Ratio(%) = Geometric Mean (Test)/Geometric Mean (Ref)
^{c}90% Confidence Interval

### Example 11. Buprenorphine Base Formulations

TABLE 77
Buprenorphine Base Formulations

| Formulation | #26 | #27 | #28 | #29 | #30 |
|---|---|---|---|---|---|
| Buprenorphine base | 0.0754 | 0.1508 | 0.3015 | 0.6030 | 1.2060 |
| BHA | 0.01 | 0.01 | 0.01 | 0.01 | 0.01 |
| BHT | 0.005 | 0.005 | 0.005 | 0.005 | 0.005 |
| L-Menthol | 0.05 | 0.05 | 0.05 | 0.05 | 0.05 |
| Ethanol | 55 | 55 | 55 | 55 | 55 |
| Propylene Glycol | 5 | 5 | 5 | 5 | 5 |
| Purified Water | 39.8596 | 39.7842 | 39.6335 | 39.332 | 38.729 |
| Citric Acid Anhydrous | QS to pH | QS to pH | QS to pH | QS to pH | QS to pH |
| Sodium Hydroxide | QS to pH | QS to pH | QS to pH | QS to pH | QS to pH |
| Nitrogen | Sparging/ | Sparging/ | Sparging/ | Sparging/ | Sparging/ |
| | Overlay | Overlay | Overlay | Overlay | Overlay |

Values = % w/w.

Formulations #15, #16 and #17 are used in the clinical trial listed as Example 8 for acute pain indication, whereas formulations #14, #15, #16, #17 and #18 will be used in chronic pain indication.

Formulations #26, #27, #28, #29 and #30 represent 0.0625 mg, 0.125 mg, 0.25 mg, 0.5 mg and 1 mg doses, respectively.

Buprenorphine formulations of Table 77 were all stable upon preparation.

### Example 12. Bioavailability Study of Buprenorphine Sublingual Sprays

The buprenorphine formulations described in Example 11, Table 77 of the instant specification were used. For formulations #27, #28 and #29 were administered as a 0.125 mg, a 0.25 mg and a 0.5 mg dose. Further, 8 mg buprenorphine tablet and 0.3 mg Buprenex® IV injections were administered.

Specifically, this was an open-label, randomized, parallel group study in healthy adult subjects. 60 subjects were divided evenly into 5 cohorts and the subject of each cohort received one of the following treatments:
Buprenorphine Sublingual Spray (1×0.5 mg sublingual dose administered every 8 hours; total of 3 doses);
Buprenorphine Sublingual Spray (1×0.25 mg sublingual dose administered every 8 hours for a total of 3 doses);
Buprenorphine Sublingual Spray (1×0.125 mg sublingual dose administered every 8 hours for total of 3 doses);
Buprenorphine Sublingual Tablet (1×8 mg sublingual dose; administered once); or
Buprenex® IV Injection (1×0.3 mg IV dose; administered every 6 hours; total of 4 doses).

### Pharmacokinetic and Bioavailability Analysis

### Method

Blood samples (1×6 mL) were collected for the quantitation of buprenorphine and norbuprenorphine at each predefined time points. The predose blood samples were collected within 60 minutes prior to the first dose of study drug. Plasma samples were analyzed using validated LC/MS/MS assays.

The following pharmacokinetic parameters were estimated from the buprenorphine and norbuprenorphine plasma concentration-time data using noncompartmental methods: areas under the curve from time 0 to the last measured concentration (AUClast), to tau (AUC0-tau), to 24 hours (AUC0-24), and to infinity (AUCinf), maximum plasma concentration (Cmax), time to reach maximum plasma concentration (Tmax), the time prior to the first measurable (non-zero) concentration (Tlag), elimination rate constant (λz), elimination half-life (t½), and dose-normalized AUCs and Cmax. Additional pharmacokinetic parameters were calculated, as appropriate. Pharmacokinetic analyses were performed using Phoenix™ WinNonlin® (Version 6.3 or higher, Pharsight Corporation). Arithmetic means, standard deviations, minimum, median, maximum, and coefficients of variation were reported. Additionally, geometric means and geometric CV % was reported for Cmax and AUCs.

### Results and Conclusions

TABLE 78
Summary of pharmacokinetic parameters of buprenorphine after administrations
of buprenorphine sublingual sprays at 0.5 mg 0.25 mg and 0.125 mg, buprenorphine
sublingual tablet at 8 mg and and Buprenex ® IV Injection at 0.3 mg.

| Parameters | 0.5 mg Spray | 0.25 mg Spray | 0.125 mg Spray | 8 mg Tablet | 0.3 mg IV Injection |
|---|---|---|---|---|---|
| Tₘₐₓ (h) | 16.8 | 13.5 | 10.0 | 2.00 | 12.1 |
| | (2.00-17.9) | (1.00-17.9) | (2.00-17.9) | (1.00-4.00) | (0.05-18.1) |
| Cₘₐₓ (ng/mL) | 1.04 | 0.501 | 0.218 | 3.80 | 23.5 |
| | (32.9%) | (31.7%) | (19.5%) | (51.3%) | (71.1%) |
| AUC₀₋₂₄ | 11.9 | 6.02 | 2.73 | 24.1 | 23.7 |
| (h*ng/mL) | (26.9%) | (29.9%) | (27.1%) | (46.3%) | (20.6%) |
| AUCₗₐₛₜ | 18.5 | 9.49 | 3.39 | 35.8 | 30.4 |
| (h*ng/mL) | (26.3%) | (33.1%) | (39.7%) | (41.0%) | (18.0%) |
| AUC_{inf} | 19.3 | 9.94 | 4.24^{a} | 37.0 | 31.3 |
| (h*ng/mL) | (27.6%) | (32.9%) | (29.7%) | (39.7%) | (17.7%) |

Note:
Tₘₐₓ presented as median (range); Cₘₐₓ and AUCs reported as mean (CV %).
NC = Not calculated; Due to n < 2, no statistics are reported.
^{a}n = 8

TABLE 79
Summary of pharmacokinetic parameters of norbuprenorphine after administrations
of buprenorphine sublingual sprays at 0.5 mg 0.25 mg and 0.125 mg, buprenorphine
sublingual tablet at 8 mg and and Buprenex ® IV Injection at 0.3 mg.

| Parameters | 0.5 mg Spray | 0.25 mg Spray | 0.125 mg Spray | 8 mg Tablet | 0.3 mg IV Injection |
|---|---|---|---|---|---|
| Tₘₐₓ (h) | 24.0 | 24.0 | 24.0 | 7.92 | 18.3 |
| | (11.9-24.1) | (11.9-48.2) | (9.00-48.0) | (1.00-48.0) | (12.3-24.0) |
| Cₘₐₓ (ng/mL) | 0.0941 | 0.0577 | 0.0377 | 0.870 | 0.0789 |
| | (38.6%) | (28.5%) | (33.9%) | (46.2%) | (44.6%) |
| AUC₀₋₂₄ | 1.20 | 0.757 | 0.394 | 13.1 | 1.03 |
| (h*ng/mL) | (47.8%) | (30.7%) | (48.5%) | (40.4%) | (42.6%) |
| AUCₗₐₛₜ | 6.61 | 3.04 | 1.17 | 43.1 | 3.62 |
| (h*ng/mL) | (49.2%) | (54.2%) | (92.6%) | (49.3%) | (52.3%) |
| AUC_{inf} | NC | NC | NC | 48.4^{a} | NC |
| (h*ng/mL) | | | | (44.5%) | |

Note:
Tₘₐₓ presented as median (range); Cₘₐₓ and AUCs reported as mean (CV %).
NC = Not calculated; Due to n < 2, no statistics are reported.
^{a}n = 10

TABLE 80
Summary of ln-transformed pharmacokinetic parameters of buprenorphine
after administrations of buprenorphine sublingual sprays at 0.5 mg
0.25 mg and 0.125 mg, and buprenorphine sublingual tablet at 8 mg.

| Parameters | 0.5 mg Spray | 0.25 mg Spray | 0.125 mg Spray | 8 mg Tablet |
|---|---|---|---|---|
| Cₘₐₓ | 29.99 | 14.54 | 6.52 | 100 |
| | (23.00-39.10) | (11.15-18.95) | (5.00-8.50) | |
| AUC₀₋₂₄ | 51.60 | 26.16 | 11.90 | 100 |
| | (41.55-64.08) | (21.07-32.49) | (9.58-14.77) | |
| AUCₗₐₛₜ | 54.10 | 27.42 | 9.56 | 100 |
| | (42.25-69.26) | (21.41-35.10) | (7.47-12.24) | |
| AUC_{inf} | 53.98 | 27.56 | 11.84 | 100 |
| | (42.56-68.47) | (21.84-34.77) | (9.13-15.36) | |
| AUC_{inf}/D₀₋₂₄ | 287.90 | 293.97 | 252.66 | 100 |
| | (226.98-365.16) | (232.98-370.92) | (194.82-327.67) | |

Note:
Cmax and AUCs reported as Geometric Mean Ratio (90% CI Lower-90% CI Upper).

TABLE 81
Summary of ln-transformed pharmacokinetic parameters of norbuprenorphine
after administrations of buprenorphine sublingual sprays at 0.5 mg 0.25
mg and 0.125 mg, and norbuprenorphine sublingual tablet at 8 mg.

| Parameters | 0.5 mg Spray | 0.25 mg Spray | 0.125 mg Spray | 8 mg Tablet |
|---|---|---|---|---|
| Cₘₐₓ | 10.97 | 7.01 | 4.53 | 100 |
| | (8.38-14.36) | (5.35-9.17) | (3.31-6.20) | |
| AUC₀₋₂₄ | 8.81 | 5.97 | 2.89 | 100 |
| | (6.45-12.04) | (4.37-8.16) | (2.01-4.16) | |
| AUCₗₐₛₜ | 14.95 | 6.89 | 2.34 | 100 |
| | (9.63-23.21) | (4.44-10.70) | (1.40-3.90) | |
| AUC_{inf} | NC | NC | NC | 100 |
| AUC_{inf}/D₀₋₂₄ | NC | NC | NC | 100 |

Note:
Cmax and AUCs reported as Geometric Mean Ratio (90% CI Lower-90% CI Upper).
NC = Not calculated; Due to n < 2, no statistics are reported.

TABLE 82
Summary of ln-transformed pharmacokinetic parameters of buprenorphine
after administrations of buprenorphine sublingual sprays at 0.5 mg
0.25 mg and 0.125 mg, and Buprenex ® IV Injection at 0.3 mg.

| Parameters | 0.5 mg Spray | 0.25 mg Spray | 0.125 mg Spray | 0.3 mg IV Injection |
|---|---|---|---|---|
| Cₘₐₓ | 5.32 | 2.58 | 1.16 | 100 |
| | (3.93-7.21) | (1.90-3.49) | (0.85-1.57) | |
| AUC₀₋₂₄ | 49.24 | 24.97 | 11.35 | 100 |
| | (41.22-58.82) | (20.90-29.83) | (9.50-13.56) | |
| AUCₗₐₛₜ | 59.67 | 30.24 | 10.55 | 100 |
| | (48.39-73.59) | (24.52-37.29) | (8.55-13.01) | |
| AUC_{inf} | 60.18 | 30.73 | 13.20 | 100 |
| | (49.47-73.21) | (25.37-37.22) | (10.66-16.36) | |

Note:
Cmax and AUCs reported as Geometric Mean Ratio (90% CI Lower-90% CI Upper).

TABLE 83
Summary of ln-transformed pharmacokinetic parameters of norbuprenorphine
after administrations of buprenorphine sublingual sprays at 0.5 mg
0.25 mg and 0.125 mg, and Buprenex ® IV Injection at 0.3 mg.

| Parameters | 0.5 mg Spray | 0.25 mg Spray | 0.125 mg Spray | 0.3 mg IV Injection |
|---|---|---|---|---|
| Cₘₐₓ | 122.23 | 78.06 | 50.45 | 100 |
| | (92.47-161.57) | (59.06-103.19) | (36.45-69.82) | |
| AUC₀₋₂₄ | 115.15 | 78.02 | 37.81 | 100 |
| | (82.61-160.51) | (55.97-108.76) | (25.68-55.67) | |
| AUCₗₐₛₜ | 193.44 | 89.16 | 30.26 | 100 |
| | (118.77-315.06) | (54.75-145.22) | (17.15-53.42) | |
| AUC_{inf} | NC | NC | NC | 100 |

After administration of Buprenorphine Sublingual Spray 0.125 to 0.5 mg Q8 h, buprenorphine was detected in systemic circulation with a mean Tlag of 0.0694 to 0.0833 hour, which was earlier than the mean Tlag observed after administration of Buprenorphine Sublingual Tablet 8 mg QD (0.174 hour).

Exposure to buprenorphine and norbuprenorphine after administration of Buprenorphine Sublingual Spray between 0.125 and 0.5 mg Q8 h was lower than that after Buprenorphine Sublingual Tablet 8 mg QD.

Ratios of the geometric means (Buprenorphine Sublingual Spray/Buprenorphine Sublingual Tablet) for buprenorphine Cmax and AUCs ranged from 6.52% (Cmax, 0.125 mg Sublingual Spray) to 54.10% (AUClast, 0.5 mg Sublingual Spray). Ratios of the geometric means (Buprenorphine Sublingual Spray/Buprenorphine Sublingual Tablet) for norbuprenorphine ranged from 2.34% (AUClast, 0.125 mg Sublingual Spray) to 14.95% (AUClast, 0.5 mg Sublingual Spray).

The geometric mean ratios for AUC0-24 were as follows:
Buprenorphine AUC0-24: 52%, 26%, and 12% for 0.5, 0.25, and 0.125 mg Buprenorphine Sublingual Spray Q8 h, respectively, compared to Buprenorphine Sublingual Tablet 8 mg single dose; and
Norbuprenorphine AUC0-24: 9%, 6%, and 3% for 0.5, 0.25, and 0.125 mg Buprenorphine Sublingual Spray Q8 h, respectively, compared to Buprenorphine Sublingual Tablet 8 mg single dose.

The geometric mean ratios (Buprenorphine Sublingual Spray/Buprenorphine Sublingual Tablet) for dose-normalized buprenorphine parameters ranged from 139.14% (Cmax/D0-24, 0.125 mg Sublingual Spray) to 293.97% (AUCinf/D0-24, 0.25 mg Sublingual Spray). Based on dose-normalized AUCinf (AUCinf/D0-24) the bioavailability of Buprenorphine Sublingual Spray relative to Buprenorphine Sublingual Tablet was 288%, 294%, and 253% for 0.5, 0.25, and 0.125 mg, respectively.

Exposure to buprenorphine after Buprenorphine Sublingual Spray between 0.125 and 0.5 mg Q8 h was lower than that after Buprenex® IV 0.3 mg Q6 h. Ratios of the geometric means (Buprenorphine Sublingual Spray/Buprenex® IV) for the primary buprenorphine parameters (Cmax, AUC0-24, AUClast, and AUCinf) ranged from 1.16% (Cmax, 0.125 mg Sublingual Spray) to 60.18% (AUCinf, 0.5 mg Sublingual Spray). Due to the lack of first pass metabolism after IV administration, the geometric mean ratios (Buprenorphine Sublingual Spray/Buprenex® IV) for norbuprenorphine were higher than observed for the parent drug and ranged from 30.26% (AUClast, 0.125 mg Sublingual Spray) to 193.44% (AUClast, 0.5 mg Sublingual Spray). The geometric mean ratios for AUC0-24 were as follows:

Buprenorphine AUC0-24: 49%, 25%, and 11% for 0.5, 0.25, and 0.125 mg Buprenorphine Sublingual Spray Q8 h, respectively, compared to Buprenex® IV 0.3 mg Q6 h.
Norbuprenorphine AUC0-24: 115%, 78%, and 38% for 0.5, 0.25, and 0.125 mg Buprenorphine Sublingual Spray Q8 h, respectively, compared to Buprenex® IV 0.3 mg Q6 h.

The geometric mean ratios (Buprenorphine Sublingual Spray/Buprenex® IV) for the primary dosenormalized buprenorphine parameters ranged from 3.70% (Cmax/D0-24, 0.125 mg Sublingual Spray) to 49.16% (AUCinf/D0-24, 0.25 mg Sublingual Spray). Based on dose-normalized AUCinf (AUCinf/D0-24) the absolute bioavailability of Buprenorphine Sublingual Spray was 48%, 49%, and 42% for 0.5, 0.25, and 0.125 mg, respectively. Thus, relative to dosage buprenorphine sublingual sprays of the present invention have a higher bioavailability than both sublingual tablets and IV injections.

### Example 13. Pharmacokinetic Study of Buprenorphine Sublingual Sprays

The buprenorphine formulations described in Example 11, Table 77 of the instant specification were used. For formulations #27, #28 and #29 were administered as a 0.125 mg, a 0.25 mg and a 0.5 mg dose. Further, 8 mg buprenorphine tablet and 0.3 mg Buprenex® IV injections were administered.

Specifically, this was an open-label, randomized, parallel group study in healthy adult subjects. 60 subjects were divided evenly into 5 cohorts and the subject of each cohort received one of the following treatments:
Buprenorphine Sublingual Spray (1×0.5 mg sublingual dose administered every 8 hours; total of 3 doses) for 5 days plus 1 dose on Day 6;
Buprenorphine Sublingual Spray (1×0.25 mg sublingual dose administered every 8 hours for a total of 3 doses) for 5 days plus 1 dose on Day 6;
Buprenorphine Sublingual Spray (1×0.125 mg sublingual dose administered every 8 hours for total of 3 doses) for 5 days plus 1 dose on Day 6;
Buprenorphine Sublingual Tablet (1×8 mg sublingual dose; administered once) for 5 days plus 1 dose on Day 6; or
Buprenex® IV Injection (1×0.3 mg IV dose; administered every 6 hours; total of 4 doses) for 5 days plus 1 dose on Day 6.

### Pharmacokinetic and Bioavailability Analysis

### Method

Blood samples (1×6 mL) were collected for the quantitation of buprenorphine and norbuprenorphine at each pre-specified time point. The predose blood samples were collected within 60 minutes prior to the first dose of study drug. Plasma samples were analyzed using validated LC/MS/MS assays. The following pharmacokinetic parameters were estimated from the buprenorphine and norbuprenorphine plasma concentration-time data using noncompartmental methods: areas under the curve from time 0 to the last measured concentration (AUClast), to tau (AUC0-tau), to 24 hours (AUC0-24), and to infinity (AUCinf), maximum plasma concentration (Cmax), time to reach maximum plasma concentration (Tmax), the time prior to the first measurable (non-zero) concentration (Tlag), elimination rate constant (λz), elimination half-life (t½), dose-normalized AUCs and Cmax, and accumulation ratios with respect to trough concentration, Cmax, and AUC0-tau.

TABLE 84
Summary of pharmacokinetic parameters of buprenorphine after administrations of
buprenorphine sublingual sprays at 0.5 mg 0.25 mg and 0.125 mg, buprenorphine sublingual
tablet at 8 mg and and Buprenex ® IV Injection at 0.3 mg on Days 1 and 6.

| Study | | 0.5 mg | 0.25 mg | 0.125 mg | 0.3 mg IV | 8 mg |
|---|---|---|---|---|---|---|
| Day | Parameters | Spray | Spray | Spray | Injection | Tablet |
| Day 1 | Tₘₐₓ (h) | 1.50 | 1.75 | 2.00 | 0.05 | 2.00 |
| | | (1.00-3.00) | (1.00-3.00) | (1.00-3.00) | (0.05-0.667) | (1.00-4.00) |
| | Cmax (ng/mL) | 0.771 | 0.380 | 0.200 | 14.2 | 4.97 |
| | | (17.9%) | (32.4%) | (23.5%) | (63.7%) | (27.6%) |
| | AUC₀₋ₜₐᵤ | 3.19 | 1.70 | 0.882 | 6.04 | 29.1 |
| | (h*ng/mL) | (14.7%) | (32.9%) | (22.5%) | (30.2%) | (24.6%) |
| | AUC₀₋₂₄ | 9.58 | 5.11 | 2.64 | 24.2 | 29.1 |
| | (h*ng/mL) | (14.7%) | (32.9%) | (22.5%) | (30.2%) | (24.6%) |
| Day 6 | Tₘₐₓ (h) | 2.00 | 2.00 | 2.00 | 0.05 | 2.00 |
| | | (1.00-4.00) | (1.00-3.00) | (1.00-2.00) | (0.05-0.0833) | (0.500-2.00) |
| | Cₘₐₓ (ng/mL) | 1.09 | 0.543 | 0.257 | 5.62 | 4.43 |
| | | (20.2%) | (23.6%) | (22.1%) | (58.2%) | (44.6%) |
| | AUC₀₋₂₄ | 17.5 | 8.66 | 4.25 | 29.2 | 35.3 |
| | (h*ng/mL) | (17.4%) | (20.2%) | (21.0%) | (29.8%) | (34.5%) |
| | AUCₗₐₛₜ | 19.8 | 10.4 | 4.21 | 24.2 | 71.1 |
| | (h*ng/mL) | (27.9%) | (34.7%) | (40.6%) | (28.2%) | (34.5%) |
| | AUC_{inf} | 21.4 | 11.6 | 4.94 | 28.2 | 76.7 |
| | (h*ng/mL) | (30.3%) | (34.2%) | (40.0%) | (29.9%) | (35.2%) |

Note:
Tmax presented as Median (Range); Cmax and AUCs reported as Mean (CV %).

TABLE 85
Summary of pharmacokinetic parameters of norbuprenorphine after administrations of
buprenorphine sublingual sprays at 0.5 mg 0.25 mg and 0.125 mg, buprenorphine
sublingual tablet at 8 mg and and Buprenex ® IV Injection at 0.3 mg on Days 1 and 6.

| Study | | 0.5 mg | 0.25 mg | 0.125 mg | 0.3 mg IV | 8 mg |
|---|---|---|---|---|---|---|
| Day | Parameters | Spray | Spray | Spray | Injection | Tablet |
| Day 1 | Tₘₐₓ (h) | 5.00 | 4.46 | 1.50 | 0.333 | 1.50 |
| | | (1.50-7.92) | (1.00-7.92) | (1.50-4.00) | (0.167-5.92) | (1.00-4.00) |
| | Cmax | 0.0520 | 0.0327 | 0.0291 | 0.0686 | 1.23 |
| | (ng/mL) | (39.1%) | (7.8%) | (23.6%) | (68.5%) | (52.1%) |
| | AUC₀₋ₜₐᵤ | 0.303^{a} | NC | NC | 0.204 | 14.6 |
| | (h*ng/mL) | (38.3%) | | | (63.1%) | (53.0%) |
| | AUC₀₋₂₄ | 0.910^{a} | NC | NC | 0.814^{b} | 14.6 |
| | (h*ng/mL) | (38.3%) | | | (63.1%) | (53.0%) |
| Day 6 | Tₘₐₓ (h) | 1.75 | 2.00 | 2.00 | 0.383 | 1.00 |
| | | (0.500-8.00) | (0.250-8.00) | (0.00-6.00) | (0.217-5.92) | (0.500-2.00) |
| | Cₘₐₓ | 0.419 | 0.186 | 0.114 | 0.350 | 3.93 |
| | (ng/mL) | (25.9%) | (49.9%) | (48.0%) | (70.8%) | (47.2%) |
| | AUC₀₋ₜₐᵤ | 2.89 | 1.33 | 0.795 | 1.79 | 60.4 |
| | (h*ng/mL) | (25.8%) | (47.0%) | (49.5%) | (73.2%) | (52.5%) |
| | AUC₀₋₂₄ | 8.67 | 4.00 | 2.38 | 7.16 | 60.4 |
| | (h*ng/mL) | (25.8%) | (47.0%) | (49.5%) | (73.2%) | (52.5%) |
| | AUCₗₐₛₜ | 19.5 | 10.5 | 5.28 | 15.6 | 173 |
| | (h*ng/mL) | (33.2%) | (73.3%) | (76.7%) | (78.7%) | (43.1%) |
| | AUC_{inf} | 22.1 | 10.7^{b} | 10.8^{a} | 14.9^{c} | 188 |
| | (h*ng/mL) | (35.1%) | (56.4%) | (31.9%) | (41.8%) | (42.4%) |

Note:
Tmax presented as Median (Range); Cmax and AUCs reported as Mean (CV %).
NC = Not calculated; Due to n < 2, no statistics are reported.
^{a}n = 5;
^{b}n = 8;
^{c}n = 7

TABLE 86
Summary of ln-transformed pharmacokinetic parameters of buprenorphine
after administrations of buprenorphine sublingual sprays at 0.5 mg 0.25
mg and Buprenex ® IV Injection at 0.3 mg on Days 1 and 6.

| | ln-transformed | | | | |
|---|---|---|---|---|---|
| Study Day | Parameters | 0.5 mg Spray | 0.25 mg Spray | 0.125 mg Spray | 0.3 mg IV Injection |
| Day 1 | Cmax | 7.08 | 3.35 | 1.81 | 100 |
| | | (5.03-9.96) | (2.38-4.72) | (1.29-2.55) | |
| | AUC0-24 | 40.90 | 20.94 | 11.13 | 100 |
| | | (34.06-49.12) | (17.44-25.15) | (9.27-13.37) | |
| | Cmax/D | 4.25 | 4.03 | 4.35 | 100 |
| | | (3.02-5.98) | (2.86-5.66) | (3.09-6.13) | |
| | AUC_{0-24/D0-24} | 32.72 | 33.51 | 35.62 | 100 |
| | | (27.25-39.30) | (27.91-40.24) | (29.66-42.78) | |
| Day 6 | Cmax | 22.38 | 11.02 | 5.25 | 100 |
| | | (17.49-28.63) | (8.57-14.17) | (4.08-6.75) | |
| | AUC0-24 | 61.40 | 30.19 | 14.80 | 100 |
| | | (52.79-71.42) | (25.87-35.23) | (12.68-17.27) | |
| | AUClast | 81.94 | 42.13 | 16.53 | 100 |
| | | (65.02-103.25) | (33.27-53.36) | (13.06-20.94) | |
| | AUC_{inf} | 76.20 | 40.62 | 16.78 | 100 |
| | | (60.31-96.27) | (31.99-51.58) | (13.22-21.31) | |
| | Cmax/D | 13.43 | 13.22 | 12.60 | 100 |
| | | (10.49-17.18) | (10.28-17.01) | (9.80-16.20) | |
| | AUC0-24/D0-24 | 49.12 | 48.30 | 47.35 | 100 |
| | | (42.23-57.13) | (41.39-56.36) | (40.58-55.25) | |
| | AUClast/D | 49.16 | 50.56 | 39.68 | 100 |
| | | (39.01-61.95) | (39.92-64.03) | (31.33-50.26) | |
| | AUCinf/D | 45.72 | 48.75 | 40.28 | 100 |
| | | (36.19-57.76) | (38.39-61.90) | (31.72-51.14) | |

Note:
Cmax and AUCs reported as Geometric Mean Ratio (%) of Test/Reference (90% CI Lower-90% CI Upper).

TABLE 87
Summary of ln-transformed pharmacokinetic parameters of norbuprenorphine
after administrations of buprenorphine sublingual sprays at 0.5 mg 0.25
mg and 0.125 mg, and Buprenex ® IV Injection at 0.3 mg on Days 1 and 6.

| | ln-transformed | | | | |
|---|---|---|---|---|---|
| Study Day | Parameters | 0.5 mg Spray | 0.25 mg Spray | 0.125 mg Spray | 0.3 mg IV Injection |
| Day 1 | Cmax | 81.53 | 54.60 | 47.64 | 100 |
| | | (59.87-111.04) | (31.58-94.40) | (29.87-75.99) | |
| | AUC0-24 | 136.08 | 93.99 | 62.80 | 100 |
| | | (65.98-280.66) | (24.45-361.41) | (16.33-241.48) | |
| | Cmax/D | 48.92 | 65.52 | 114.34 | 100 |
| | | (35.92-66.62) | (37.89-113.28) | (71.68-182.37) | |
| | AUC_{0-24/D0-24} | 108.87 | 150.39 | 200.97 | 100 |
| | | (52.79-224.53) | (39.11-578.25) | (52.27-772.72) | |
| Day 6 | Cmax | 135.83 | 57.45 | 34.00 | 100 |
| | | (98.84-186.65) | (41.52-79.49) | (24.57-47.05) | |
| | AUC0-24 | 138.12 | 60.95 | 34.83 | 100 |
| | | (100.73-189.38) | (44.15-84.15) | (25.23-48.08) | |
| | AUClast | 142.64 | 67.78 | 28.86 | 100 |
| | | (90.73-224.25) | (42.69-107.60) | (18.18-45.81) | |
| | AUC_{inf} | 153.27 | 71.18 | 74.97 | 100 |
| | | (108.58-216.37) | (48.91-103.59) | (49.04-114.62) | |
| | Cmax/D | 81.50 | 68.94 | 81.60 | 100 |
| | | (59.31-111.99) | (49.82-95.38) | (58.98-112.91) | |
| | AUC0-24/D0-24 | 110.49 | 97.53 | 111.46 | 100 |
| | | (80.58-151.50) | (70.65-134.63) | (80.74-153.87) | |
| | AUClast/D | 85.58 | 81.34 | 69.25 | 100 |
| | | (54.44-134.55) | (51.23-129.13) | (43.62-109.95) | |
| | AUCinf/D | 91.96 | 85.42 | 179.93 | 100 |
| | | (65.15-129.82) | (58.70-124.30) | (117.70-275.08) | |

Note:
Cmax and AUCs reported as Geometric Mean Ratio (%) of Test/Reference (90% CI Lower-90% CI Upper).

TABLE 88
Summary of ln-transformed pharmacokinetic parameters of buprenorphine after
administrations of buprenorphine sublingual sprays at 0.5 mg 0.25 mg and
0.125 mg, and buprenorphine sublingual tablet at 8 mg on Days 1 and 6.

| | ln-transformed | | | | |
|---|---|---|---|---|---|
| Study Day | Parameters | 0.5 mg Spray | 0.25 mg Spray | 0.125 mg Spray | 8 mg Tablet |
| Day 1 | Cmax | 15.88 | 7.53 | 4.07 | 100 |
| | | (13.22-19.08) | (6.26-9.04) | (3.39-4.89) | |
| | AUC0-24 | 33.50 | 17.16 | 9.12 | 100 |
| | | (28.20-39.81) | (14.44-20.38) | (7.67-10.83) | |
| | Cmax/D | 254.14 | 240.83 | 260.46 | 100 |
| | | (211.51-305.35) | (200.44-289.36) | (216.78-312.94) | |
| | AUC_{0-24/D0-24} | 178.69 | 182.99 | 194.52 | 100 |
| | | (150.40-212.31) | (154.02-217.42) | (163.71-231.11) | |
| Day 6 | Cmax | 26.55 | 13.08 | 6.23 | 100 |
| | | (21.59-32.65) | (10.59-16.15) | (5.04-7.69) | |
| | AUC0-24 | 51.82 | 25.48 | 12.49 | 100 |
| | | (43.64-61.55) | (21.38-30.37) | (10.48-14.89) | |
| | AUClast | 28.73 | 14.77 | 5.80 | 100 |
| | | (22.38-36.88) | (11.45-19.07) | (4.49-7.48) | |
| | AUC_{inf} | 28.76 | 15.34 | 6.33 | 100 |
| | | (22.38-36.96) | (11.87-19.81) | (4.90-8.18) | |
| | Cmax/D | 424.87 | 418.40 | 398.66 | 100 |
| | | (345.50-522.47) | (338.73-516.81) | (322.74-492.42) | |
| | AUC0-24/D0-24 | 276.40 | 271.77 | 266.42 | 100 |
| | | (232.74-328.25) | (228.00-323.95) | (223.51-317.56) | |
| | AUClast/D | 459.67 | 472.74 | 371.04 | 100 |
| | | (358.10-590.05) | (366.31-610.09) | (287.51-478.85) | |
| | AUCinf/D | 460.23 | 490.72 | 405.44 | 100 |
| | | (358.14-591.42) | (379.82-634.02) | (313.80-523.83) | |

Note:
Cmax and AUCs reported as Geometric Mean Ratio (%) of Test/Reference (90% CI Lower-90% CI Upper).

TABLE 89
Summary of ln-transformed pharmacokinetic parameters of norbuprenorphine after
administrations of buprenorphine sublingual sprays at 0.5 mg 0.25 mg and 0.125
mg, and buprenorphine sublingual tablet at 8 mg and on Days 1 and 6.

| | ln-transformed | | | | |
|---|---|---|---|---|---|
| Study Day | Parameters | 0.5 mg Spray | 0.25 mg Spray | 0.125 mg Spray | 8 mg Tablet |
| Day 1 | Cmax | 5.04 | 3.37 | 2.94 | 100 |
| | | (3.20-7.94) | (1.44-7.89) | (1.44-6.04) | |
| | AUC0-24 | 7.50 | 5.18 | 3.46 | 100 |
| | | (3.64-15.46) | (1.26-21.30) | (0.84-14.23) | |
| | Cmax/D | 80.63 | 107.98 | 188.45 | 100 |
| | | (51.20-126.99) | (46.17-252.58) | (91.90-386.44) | |
| | AUC_{0-24/D0-24} | 40.00 | 55.25 | 73.84 | 100 |
| | | (19.41-82.43) | (13.44-227.21) | (17.96-303.62) | |
| Day 6 | Cmax | 11.34 | 4.80 | 2.84 | 100 |
| | | (8.43-15.26) | (3.54-6.50 ) | (2.10-3.85) | |
| | AUC0-24 | 15.33 | 6.77 | 3.87 | 100 |
| | | (11.43-20.57) | (5.01-9.13) | (2.86-5.22) | |
| | AUClast | 11.49 | 5.46 | 2.32 | 100 |
| | | (7.51-17.57) | (3.54-8.43) | (1.51-3.59) | |
| | AUC_{inf} | 11.89 | 5.52 | 5.81 | 100 |
| | | (8.96-15.78) | (4.03-7.57) | (4.03-8.38) | |
| | Cmax/D | 181.45 | 153.49 | 181.69 | 100 |
| | | (134.81-244.23) | (113.31-207.92) | (134.13-246.13) | |
| | AUC0-24/D0-24 | 81.76 | 72.17 | 82.48 | 100 |
| | | (60.95-109.69) | (53.46-97.43) | (61.09-111.35) | |
| | AUClast/D | 183.79 | 174.67 | 148.72 | 100 |
| | | (120.13-281.18) | (113.13-269.68) | (96.32-229.63) | |
| | AUCinf/D | 190.21 | 176.66 | 372.15 | 100 |
| | | (143.29-252.48) | (128.89-242.14) | (258.10-536.60) | |

Note:
Cmax and AUCs reported as Geometric Mean Ratio (%) of Test/Reference (90% CI Lower-90% CI Upper).

### Results and Conclusions

After administration of Buprenorphine Sublingual Spray, concentrations increased with dose and peak buprenorphine concentrations were observed at a median Tmax between 1.50 and 2.00 h, similar to the time to reach maximum buprenorphine concentration after administration of Buprenorphine Sublingual Tablet.

On Day 1 and Day 6, exposure to buprenorphine after administration of Buprenorphine Sublingual Spray between 0.125 and 0.5 mg Q8 h was lower than that after Buprenex IV 0.3 mg Q6 h. Exposure to norbuprenorphine was lower than that after Buprenex IV 0.3 mg Q6 h only for Buprenorphine Sublingual Spray 0.125 and 0.25 mg Q8 h. The geometric mean ratios for AUC0-24 were:
Buprenorphine AUC0-24 on Day 6: 61%, 30%, and 15% for 0.5, 0.25, and 0.125 mg;
Buprenorphine Sublingual Spray Q8 h, respectively, compared to Buprenex IV 0.3 mg Q6 h;
Norbuprenorphine AUC0-24 on Day 6: 138%, 61%, and 35% for 0.5, 0.25, and 0.125 mg; and
Buprenorphine Sublingual Spray Q8 h, respectively, compared to Buprenex IV 0.3 mg Q6 h.

Based on dose-normalized AUCinf on Day 6, the absolute bioavailability of Buprenorphine Sublingual Spray was 46%, 49%, and 40% for 0.5, 0.25, and 0.125 mg, respectively.

On Day 1 and Day 6, exposure to buprenorphine and norbuprenorphine after administration of Buprenorphine Sublingual Spray between 0.125 and 0.5 mg Q8 h was lower than that after Buprenorphine Sublingual Tablet 8 mg QD. The geometric mean ratios for AUC0-24 were:
Buprenorphine AUC0-24 on Day 6: 52%, 25%, and 12% for 0.5, 0.25, and 0.125 mg;
Buprenorphine Sublingual Spray Q8 h, respectively, compared to Buprenorphine Sublingual Tablet 8 mg QD;
Norbuprenorphine AUC0-24 on Day 6: 15%, 7%, and 4% for 0.5, 0.25, and 0.125 mg; and
Buprenorphine Sublingual Spray Q8 h, respectively, compared to Buprenorphine Sublingual Tablet 8 mg QD.

Based on dose-normalized AUCinf on Day 6, the bioavailability of Buprenorphine Sublingual Spray relative to Buprenorphine Sublingual Tablet was 460%, 491%, and 405% for 0.5, 0.25, and 0.125 mg, respectively.

During dosing of Buprenorphine Sublingual Spray Q8 h over 6 days, accumulation of buprenorphine in systemic circulation was slightly higher than the accumulation observed after Buprenex IV 0.3 mg Q6 h and Buprenorphine Sublingual Tablet 8 mg QD. The metabolite-to-parent ratios after Buprenorphine Sublingual Spray Q8 h were higher than observed after Buprenex IV 0.3 mg Q6 h but lower than observed after Buprenorphine Sublingual Tablet 8 mg QD. During dosing of Buprenorphine Sublingual Spray Q8 h, steady state buprenorphine concentrations were achieved between Day 3 and Day 5, approximately the same time as for Buprenex IV 0.3 mg Q6 h and Buprenorphine Sublingual Tablet 8 mg QD. Trough concentrations of norbuprenorphine on Day 5 and Day 6 were similar. Buprenorphine exposure is proportional to dose for Buprenorphine Sublingual Spray between 0.25 and 0.5 mg, for both single administration and after multiple dosing Q8 h.

### Example 14. Bioavailability Study of Buprenorphine Sublingual Sprays

The buprenorphine formulations described in Example 11, Table 77 of the instant specification were used. For formulations #26, #27, #28, #29 and #30 were administered as a 0.0625 mg, 0.125 mg, a 0.25 mg, a 0.5 and a 1.0 mg dose.

Specifically, a single dose, open-label, randomized, parallel group study to compare the bioavailability of five single doses of Buprenorphine Sublingual Spray. 30 subjects were divided evenly into 5 cohorts and the subjects of each cohort received one of the following treatments:

0.0625 mg, 1 spray, administered sublingually;

0.125 mg, 1 spray, administered sublingually;

0.25 mg, 1 spray, administered sublingually;

0.5 mg, 1 spray, administered sublingually; and

1.0 mg, 1 spray, administered sublingually.

### Pharmacokinetic and Bioavailability Analysis

### Method

Blood samples (1×6 mL) for buprenorphine and norbuprenorphine analyses were collected in Vacutainer tubes containing K2-EDTA at 0 hour (predose), 5 minutes, 10 minutes, 15 minutes, 30 minutes, and 1, 1.5, 2, 3, 4, 6, 8, 12, 24, 48, 72, 96, 120, and 144 hours post dose (19 time points) for quantification of buprenorphine and norbuprenorphine. Subjects who experienced emesis within 2 hours of administration of the study drug were not included in the pharmacokinetic and statistical analyses.

Single-dose pharmacokinetic parameters for buprenorphine and norbuprenorphine were calculated using non-compartmental techniques in Phoenix™ WinNonlin® (Version 6.3, Pharsight Corporation). Concentration-time data that were below the limit of quantification (BLQ) were treated as zero in the data summarization and descriptive statistics. In the pharmacokinetic analysis, BLQ concentrations were treated as zero from time-zero up to the time at which the first quantifiable concentration was observed; embedded and/or terminal BLQ concentrations were treated as “missing”. Actual sample times were used for all pharmacokinetic and statistical analyses. The linear log trapezoidal method was used to calculate the area under the curve (AUC).

The following pharmacokinetic parameters were calculated: peak concentration in plasma (Cmax), time to peak concentration (Tmax), elimination rate constant (λz), terminal half-life (t½), area under the concentration-time curve from time-zero to the time of the last quantifiable concentration (AUClast), area under the plasma concentration time curve from time-zero extrapolated to infinity (AUCinf), the percent of AUCinf based on extrapolation (AUCextrap), last quantifiable plasma concentration (Clast), and time of the last quantifiable plasma concentration (Tlast).

TABLE 90
Summary of pharmacokinetic parameters of buprenorphine after administrations of
buprenorphine sublingual sprays at 1.0 mg, 0.5 mg 0.25 mg, 0.125 mg and 0.0625 mg
buprenorphine.

| | 0.0625 mg | | | 0.125 mg | | |
|---|---|---|---|---|---|---|
| Parameter | Mean | SD | CV % | Mean | SD | CV % |
| Tₘₐₓ (h) | 1.50 (0.50-2.00) | | | 1.50 (1.00-2.00) | | |
| Cₘₐₓ (ng/mL) | 0.125 | 0.0203 | 16.22 | 0.215 | 0.0463 | 21.58 |
| Cₘₐₓ/Dose | 2.01 | 0.325 | 16.22 | 1.72 | 0.370 | 21.58 |
| ((ng/mL)/mg) | | | | | | |
| AUCₗₐₛₜ (h*ng/mL) | 0.5741 | 0.1721 | 29.98 | 1.046 | 0.3555 | 33.99 |
| AUCₗₐₛₜ/Dose | 9.186 | 2.753 | 29.98 | 8.368 | 2.844 | 33.99 |
| ((h*ng/mL)/mg) | | | | | | |
| AUC_{inf} (h*ng/mL) | 0.6387 | 0.1844 | 28.88 | 1.126 | 0.3674 | 32.62 |
| AUC_{inf}/Dose | 10.22 | 2.951 | 28.88 | 9.011 | 2.939 | 32.62 |
| ((h*ng/mL)/mg) | | | | | | |
| AUC_{Extrap} (%) | 10.44 | 2.66 | 25.49 | 7.41 | 1.42 | 19.15 |
| λ_{z} (h⁻¹) | 0.2840 | 0.0716 | 25.23 | 0.2058 | 0.0410 | 19.92 |
| T_{1/2} (h) | 2.57 | 0.61 | 23.75 | 3.52 | 0.93 | 26.41 |
| Tₗₐₛₜ (h) | 9.67 | 2.66 | 27.50 | 14.41 | 5.36 | 37.22 |
| Cₗₐₛₜ (ng/mL) | 0.0175 | 0.00385 | 22.07 | 0.0161 | 0.00176 | 10.96 |
| | 0.25 mg | | | | | |
| Parameter | Mean | | SD | | CV % | |
| Tₘₐₓ (h) | 1.25 (1.00-2.00) | | | | | |
| Cₘₐₓ (ng/mL) | 0.385 | | 0.126 | | 32.65 | |
| Cₘₐₓ/Dose | 1.54 | | 0.502 | | 32.65 | |
| ((ng/mL)/mg) | | | | | | |
| AUCₗₐₛₜ (h*ng/mL) | 2.215 | | 0.8010 | | 36.17 | |
| AUCₗₐₛₜ/Dose | | | | | | |
| ((h*ng/mL)/mg) | 8.859 | | 3.204 | | 36.17 | |
| AUC_{inf} (h*ng/mL) | 2.277 | | 0.9231 | | 40.55 | |
| AUC_{inf}/Dose | 9.106 | | 3.693 | | 40.55 | |
| ((h*ng/mL)/mg) | | | | | | |
| AUC_{Extrap} (%) | 6.40 | | 1.63 | | 25.46 | |
| λ_{z} (h⁻¹) | 0.1349 | | 0.0765 | | 56.74 | |
| T_{1/2} (h) | 6.89 | | 4.64 | | 67.38 | |
| Tₗₐₛₜ (h) | 30.00 | | 14.70 | | 48.99 | |
| Cₗₐₛₜ (ng/mL) | 0.0155 | | 0.00458 | | 29.52 | |
| | 0.5 mg | | | 1.0 mg | | |
| parameters | Mean | SD | CV % | Mean | SD | CV % |
| Tₘₐₓ (h) | 1.75 (1.00-2.00) | | | 1.50 (1.00-1.50) | | |
| Cₘₐₓ (ng/mL) | 0.865 | | 32.08 | 1.57 | 0.453 | 28.82 |
| Cₘₐₓ/Dose | 1.73 | 0.555 | 32.08 | 1.57 | 0.453 | 28.82 |
| (ng/mL)/mg) | | | | | | |
| AUCₗₐₛₜ (h*ng/mL) | 5.023 | 1.242 | 24.72 | 10.68 | 3.096 | 29.00 |
| AUCₗₐₛₜ/Dose | 10.05 | 2.483 | 24.72 | 10.68 | 3.096 | 29.00 |
| (h*ng/mL)/mg) | | | | | | |
| AUC_{inf} (h*ng/mL) | 5.366 | 1.724 | 32.13 | 11.36 | 3.153 | 27.76 |
| AUC_{inf}/Dose | 10.73 | 3.448 | 32.13 | 11.36 | 3.153 | 27.76 |
| (h*ng/mL)/mg) | | | | | | |
| AUC_{Extrap} (%) | 6.88 | 3.62 | 52.58 | 6.36 | 1.98 | 31.08 |
| λ_{z} (h⁻¹) | 0.0606 | 0.0416 | 68.74 | 0.0222 | 0.0042 | 19.05 |
| T_{1/2} (h) | 18.28 | 15.38 | 84.10 | 32.17 | 6.15 | 19.12 |
| Tₗₐₛₜ (h) | 52.01 | 23.62 | 45.42 | 100.00 | 18.07 | 18.07 |
| Cₗₐₛₜ (ng/mL) | 0.0158 | 0.00364 | 23.00 | 0.0148 | 0.00207 | 14.01 |

Note:
Tmax reported as Median (Range).
NC = Not calculated

TABLE 91
Summary of pharmacokinetic parameters of norbuprenorphine after
administrations of buprenorphine sublingual sprays at 1.0 mg, 0.5 mg
0.25 mg, 0.125 mg and 0.0625 mg buprenorphine.

| | 0.25 mg | | | 0.5 mg | | |
|---|---|---|---|---|---|---|
| Parameter | Mean | SD | CV % | Mean | SD | CV % |
| Tₘₐₓ (h) | 2.00 (1.00-8.00) | | | 2.00 (2.00-24.00 | | |
| Cₘₐₓ (ng/mL) | 0.0367 | 0.0121 | 32.84 | 0.0601 | 0.0331 | 55.07 |
| Cₘₐₓ/Dose | 0.147 | 0.0482 | 32.84 | 0.120 | 0.0662 | 55.07 |
| ((ng/mL)/mg) | | | | | | |
| AUCₗₐₛₜ (h*ng/mL) | 0.764 | 0.6291 | 82.73 | 1.944 | 1.891 | 97.31 |
| AUCₗₐₛₜ/Dose | 3.042 | 2.516 | 82.73 | 3.887 | 3.783 | 97.31 |
| ((h*ng/mL)/mg) | | | | | | |
| AUC_{inf} (n*ng/mL) | NC | | | 7.409 | NC | NC |
| AUC_{inf}/Dose | NC | | | 14.82 | NC | NC |
| ((h*ng/mL)/mg) | | | | | | |
| AUC_{Extrap} (%) | NC | | | 28.8 | NC | NC |
| λ_{z} (h⁻¹) | NC | | | 0.0120 | NC | NC |
| T_{1/2} (h) | NC | | | 57.77 | NC | NC |
| Tₗₐₛₜ (h) | 28.00 | 18.33 | 65.47 | 52.8 | 39.44 | 74.69 |
| Cₗₐₛₜ (ng/mL) | 0.0220 | 0.00196 | 8.90 | 0.0287 | 0.009458 | 33.41 |

| | 1.0 mg | | |
|---|---|---|---|
| Parameter | Mean | SD | CV % |
| Tₘₐₓ (h) | 3.75 (1.50-8.08) | | |
| Cₘₐₓ (ng/mL) | 0.0990 | 0.0512 | 51.68 |
| Cₘₐₓ/Dose | 0.0990 | 0.0512 | 51.68 |
| ((ng/mL)/mg) | | | |
| AUCₗₐₛₜ (h*ng/mL) | 5.345 | 4.283 | 80.12 |
| AUCₗₐₛₜ/Dose | 5.345 | 4.283 | 80.12 |
| ((h*ng/mL)/mg) | | | |
| AUC_{inf} (h*ng/mL) | 15.56 | NC | NC |
| AUC_{inf}/Dose | 15.56 | NC | NC |
| ((h*ng/mL)/mg) | | | |
| AUC_{Extrap} (%) | 16.78 | NC | NC |
| λ_{z} (h⁻¹) | 0.0126 | NC | NC |
| T_{1/2} (h) | 54.84 | NC | NC |
| Tₗₐₛₜ (h) | 96.00 | 42.93 | 44.72 |
| Cₗₐₛₜ (ng/mL) | 0.0250 | 0.00424 | 16.95 |

Note:
Tmax reported as Median (Range).
NC = Not calculated

### Results and Conclusions

### Buprenorphine

After single dose administrations of Buprenorphine Sublingual Spray, maximum buprenorphine exposure occurred between 1.25 h and 1.75 h postdose. Mean dose-normalized buprenorphine Cmax values were similar across treatments, ranging from 1.54 ng/mL/mg (Treatment C; 0.25 mg) to 2.01 ng/mL/mg (Treatment A; 0.0625 mg). Mean dose-normalized buprenorphine AUClast and AUCinf values were similar across treatments as well, ranging from 8.368 h*ng/mL/mg (Treatment B; 0.125 mg) to 10.68 h*ng/mL/mg (Treatment E; 1.0 mg) for AUClast/Dose and from 9.011 h*ng/mL/mg (Treatment B; 0.125 mg) to 11.36 h*ng/mL/mg (Treatment E; 1.0 mg) for AUCinf/Dose. From the analysis of dose proportionality for buprenorphine, the slopes ranged from 0.9154 (Cmax) to 1.0721 (AUClast), suggesting that the increase in maximum and total buprenorphine exposure was dose-proportional from 0.0625 mg to 1.0 mg.

### Norbuprenorphine

After single dose administrations of Buprenorphine Sublingual Spray, maximum norbuprenorphine exposure occurred between 2.00 h and 3.75 h postdose. Mean dose-normalized norbuprenorphine Cmax values decreased with an increase in dose, ranging from 0.147 ng/mL/mg (Treatment C; 0.25 mg) to 0.0990 ng/mL/mg (Treatment E; 1.0 mg). Mean dose-normalized norbuprenorphine AUClast values increased with an increase in dose, ranging from 3.042 h*ng/mL/mg (Treatment C; 0.25 mg) to 5.345 h*ng/mL/mg (Treatment E; 1.0 mg). From the analysis of dose proportionality for norbuprenorphine, the slope for Cmax was 0.6418, indicating that norbuprenorphine Cmax increased in a less than proportional manner with an increase in dose from 0.25 mg to 1.0 mg. The slope for AUClast was 1.8594, suggesting that norbuprenorphine AUClast increased in a greater than proportional manner with an increase in dose from 0.25 mg to 1.0 mg.

### Example 15. Study of the Effects of Oral Cavity Temperature and pH on the Bioavailability of Buprenorphine Sublingual Sprays

The buprenorphine formulations described in Example 11, Table 77 of the instant specification were used. For formulation #29 was administered as a 0.5 mg dose.

Specifically, a single dose, open-label, randomized, five-period, crossover study to compare the bioavailability of single doses of Buprenorphine Sublingual Spray under various conditions. Each dose of study treatment was separated by a washout period of 7 days. 15 subjects were divided evenly into 5 cohorts and the subjects of each cohort received treatments under the following conditions according to the order in Table 92:

A=Subject given cold water approximately 1 minute before study treatment;

B=Subject given hot water approximately 1 minute before study treatment;

C=Subject given no pretreatment beverage (the reference treatment);

D=Subject given low pH beverage approximately 1 minute before study treatment; and

E=Subject given high pH beverage approximately 1 minute before study treatment.

TABLE 92
Order of Treatment Conditions for Each Cohort.

| Cohort | Period 1 | Period 2 | Period 3 | Period 4 | Period 5 |
|---|---|---|---|---|---|
| 1 | A | B | E | C | D |
| 2 | B | C | A | D | E |
| 3 | C | D | B | E | A |
| 4 | D | E | C | A | B |
| 5 | E | A | D | B | C |

### Pharmacokinetic and Bioavailability Analysis

### Method

In each study period, blood samples were collected for the quantitation of buprenorphine and norbuprenorphine at 0 (predose), at 5 minutes, 10 minutes, 15 minutes, 30 minutes, and 1, 2, 4, 8, 12, 24, 48, 72, 96, 120, and 144 hours post dose (16 time points).

Single-dose pharmacokinetic parameters for buprenorphine and norbuprenorphine were calculated using non-compartmental techniques in Phoenix™ WinNonlin® (Version 6.4, Pharsight Corporation). Concentration-time data that were below the limit of quantification (BLQ) were treated as zero in the data summarization and descriptive statistics. In the pharmacokinetic analysis, BLQ concentrations were treated as zero from time-zero up to the time at which the first quantifiable concentration was observed; embedded and/or terminal BLQ concentrations were treated as “missing”. Actual sample times were used for all pharmacokinetic and statistical analyses. The linear log trapezoidal method was used to calculate the area under the curve (AUC).

The following pharmacokinetic parameters were calculated: peak concentration in plasma (Cmax), time to peak concentration (Tmax), elimination rate constant (λz), terminal half-life (t½), area under the concentration-time curve from time-zero to the time of the last quantifiable concentration (AUClast), area under the plasma concentration time curve from time-zero extrapolated to infinity (AUC0-inf), the percent of AUCinf based on extrapolation (AUCextrap), last quantifiable plasma concentration (Clast), and time of the last quantifiable plasma concentration (Tlast).

TABLE 93
Summary of pharmacokinetic parameters of buprenorphine after administrations
of buprenorphine under treatment conditions A, B, C, D and E.

| | A (Cold Water) | | | B (Hot Water) | | |
|---|---|---|---|---|---|---|
| Parameter | Mean | SD | CV % | Mean | SD | CV % |
| Tₘₐₓ (h) | 2.00 (0.500-2.07) | | | 2.00 (1.00-4.00) | | |
| Cₘₐₓ (ng/mL) | 0.730 | 0.243 | 33.31 | 0.839 | 0.259 | 30.88 |
| AUCₗₐₛₜ (h*ng/mL) | 5.00 | 1.67 | 33.42 | 5.84 | 1.78 | 30.45 |
| AUC_{inf} (h*ngimL) | 5.74 | 1.72 | 29.88 | 6.08 | 2.01 | 33.11 |
| AUC_{Extrap} (%) | 6.82 | 1.34 | 19.64 | 6.62 | 1.03 | 15.54 |
| λ_{z} (h⁻¹) | 0.0461 | 0.0232 | 50.23 | 0.0467 | 0.0261 | 55.86 |
| T_{1/2} (h) | 17.5 | 6.44 | 36.86 | 18.2 | 7.43 | 40.90 |
| Tₗₐₛₜ (h) | 49.6 | 19.2 | 38.65 | 57.6 | 21.8 | 37.93 |
| Cₗₐₛₜ (ng/mL) | 0.0170 | 0.00343 | 20.25 | 0.0166 | 0.00371 | 22.38 |
| | C (no pretreatment) | | | | | |
| Parameter | Mean | | SD | | CV % | |
| Tₘₐₓ (h) | 2.00 (0.500-4.00) | | | | | |
| Cₘₐₓ (ng/mL) | 0.766 | | 0.244 | | 31.85 | |
| AUCₗₐₛₜ (h*ng/mL) | 5.42 | | 1.59 | | 29.36 | |
| AUC_{inf} (h*ng/mL) | 5.79 | | 1.66 | | 28.61 | |
| AUC_{Extrap} (%) | 6.94 | | 1.41 | | 20.33 | |
| λ_{z} (h⁻¹) | 0.0525 | | 0.0283 | | 53.99 | |
| T_{1/2} (h) | 16.3 | | 6.96 | | 42.61 | |
| Tₗₐₛₜ (h) | 54.4 | | 23.1 | | 42.40 | |
| Cₗₐₛₜ (ng/mL) | 0.0181 | | 0.00554 | | 30.57 | |
| | D (Low pH) | | | E (High pH) | | |
| parameters | Mean | SD | CV % | Mean | SD | CV % |
| Tₘₐₓ (h) | 2.00 (0.50-4.00) | | | 2.00 (1.00-4.00) | | |
| Cₘₐₓ (ng/mL) | 0.705 | 0.196 | 27.78 | 0.764 | 0.246 | 32.14 |
| AUCₗₐₛₜ (h*ng/mL) | 5.16 | 1.68 | 32.55 | 5.65 | 2.06 | 36.50 |
| AUC_{inf} (h*hg/mL) | 5.70 | 1.45 | 25.37 | 5.87 | 2.12 | 36.21 |
| AUC_{Extrap} (%) | 7.22 | 1.05 | 14.56 | 6.87 | 1.46 | 21.30 |
| λ_{z} (h⁻¹) | 0.0448 | 0.0249 | 55.56 | 0.0559 | 0.0297 | 53.14 |
| T_{1/2} (h) | 19.0 | 8.57 | 45.19 | 16.0 | 8.59 | 53.80 |
| Tₗₐₛₜ (h) | 53.1 | 21.4 | 40.31 | 56.0 | 23.4 | 41.82 |
| Cₗₐₛₜ (ng/mL) | 0.0174 | 0.00370 | 21.22 | 0.0179 | 0.00514 | 28.69 |

TABLE 94
Summary of pharmacokinetic parameters of norbuprenorphine after administrations
of buprenorphine under treatment conditions A, B, C, D and E.

| | A (Cold Water) | | | B (Hot Water) | | |
|---|---|---|---|---|---|---|
| Parameter | Mean | SD | CV % | Mean | SD | CV % |
| Tₘₐₓ (h) | 4.00 (1.00-24.0) | | | .00 (0.500-8.00) | | |
| Cₘₐₓ (ng/mL) | 0.0686 | 0.0272 | 39.64 | 0.0722 | 0.259 | 30.88 |
| AUCₗₐₛₜ (h*ng/mL) | 2.25 | 1.39 | 61.62 | 2.78 | 1.78 | 30.45 |
| AUC_{inf} (h*ng/mL) | NC | NC | NC | NC | NC | NC |
| AUC_{Extrap} (%) | NC | NC | NC | NC | NC | NC |
| λ_{z} (h⁻¹) | NC | NC | NC | NC | NC | NC |
| T_{1/2} (h) | NC | NC | NC | NC | NC | NC |
| Tₗₐₛₜ (h) | 53.6 | 27.5 | 51.38 | 64.0 | 33.5 | 52.40 |
| Cₗₐₛₜ (ng/mL) | 0.0254 | 0.0380 | 14.95 | 0.0249 | 0.00362 | 14.54 |
| | C (no pretreatment) | | | | | |
| Parameter | Mean | | SD | | CV % | |
| Tₘₐₓ (h) | 4.00 (1.00-24.0) | | | | | |
| Cₘₐₓ (ng/mL) | 0.0664 | | 0.0245 | | 36.88 | |
| AUCₗₐₛₜ (h*ng/mL) | 2.34 | | 1.33 | | 56.63 | |
| AUC_{inf} (h*ng/mL) | NC | | NC | | NC | |
| AUC_{Extrap} (%) | NC | | NC | | NC | |
| λ_{z} (h⁻¹) | NC | | NC | | NC | |
| T_{1/2} (h) | NC | | NC | | NC | |
| Tₗₐₛₜ (h) | 55.2 | | 26.4 | | 47.77 | |
| Cₗₐₛₜ (ng/mL) | 0.0255 | | 0.00546 | | 21.38 | |
| | D (Low pH) | | | E (High pH) | | |
| parameters | Mean | SD | CV % | Mean | SD | CV % |
| Tₘₐₓ (h) | 2.00 (1.00-24.0) | | | 4.00 (1.00-24.0) | | |
| Cₘₐₓ (ng/mL) | 0.6906 | 0.0261 | 37.53 | 0.637 | 0.0250 | 39.24 |
| AUCₗₐₛₜ (h*ng/mL) | 2.30 | 1.51 | 65.56 | 2.04 | 1.19 | 58.21 |
| AUC_{inf} (h*ng/mL) | 5.58 | 0.00 | 0.00 | NC | NC | NC |
| AUC_{Extrap} (%) | 18.9 | 0.00 | 0.00 | NC | NC | NC |
| λ_{z} (h⁻¹) | 0.0193 | 0.00 | 0.00 | NC | NC | NC |
| T_{1/2} (h) | 36.0 | 0.00 | 0.00 | NC | NC | NC |
| Tₗₐₛₜ (h) | 58.2 | 33.2 | 57.04 | 53.6 | 29.0 | 54.09 |
| Cₗₐₛₜ (ng/mL) | 0.0243 | 0.00428 | 17.62 | 0.0262 | 0.00806 | 30.81 |

TABLE 95
Statistical Analysis of the Log-transformed Systemic Exposure Parameters of Buprenorphine.

| Dependent | Geometric Mean | | Ratio (%) | 90% CI^{c} | | | ANOVA |
|---|---|---|---|---|---|---|---|
| Variable | A | C | (A/C) | Lower | Upper | Power | CV % |
| ln(Cₘₐₓ) | 0.6867 | 0.7299 | 94.08 | 83.79 | 105.62 | 0.936 | 19.09 |
| ln(AUCₗₐₛₜ) | 4.6967 | 5.1933 | 90.44 | 81.42 | 100.46 | 0.967 | 17.30 |
| ln(AUCi_{nf}) | 5.0347 | 4.7971 | 104.95 | 93.43 | 117.89 | 0.935 | 15.64 |

Pre-Treated with Hot Water vs. Without Pre-Treatment

| Dependent | Geometric Mean^{a} | | Ratio (%)^{b} | 90% CI^{c} | | | ANOVA |
|---|---|---|---|---|---|---|---|
| Variable | B | C | (B/C) | Lower | Upper | Power | CV % |
| ln(Cₘₐₓ) | 0.8028 | 0.7299 | 109.99 | 97.96 | 123.48 | 0.936 | 19.09 |
| ln(AUCₗₐₛₜ) | 5.5598 | 5.1933 | 107.06 | 96.38 | 118.92 | 0.967 | 17.30 |
| ln(AUC_{inf}) | 5.5833 | 4.7971 | 116.39 | 104.06 | 130.18 | 0.948 | 15.64 |

Pre-Treated with Low pH Beverage vs. Without Pre-Treatment

| Dependent | Geometric Mean^{a} | | Ratio (%)^{b} | 90% CI^{c} | | | ANOVA |
|---|---|---|---|---|---|---|---|
| Variable | D | C | (D/C) | Lower | Upper | Power | CV % |
| ln(Cₘₐₓ) | 0.6555 | 0.7299 | 89.81 | 79.78 | 101.11 | 0.927 | 19.09 |
| ln(AUCₗₐₛₜ) | 4.7159 | 5.1933 | 90.81 | 81.55 | 101.12 | 0.961 | 17.30 |
| ln(AUC_{inf}) | 4.5199 | 4.7971 | 94.22 | 83.07 | 106.86 | 0.900 | 15.64 |

Pre-Treated with High pH Beverage vs. Without Pre-Treatment

| Dependent | Geometric Mean^{a} | | Ratio (%)^{b} | 90% CI^{c} | | | ANOVA |
|---|---|---|---|---|---|---|---|
| Variable | E | C | (E/C) | Lower | Upper | Power | CV % |
| ln(Cₘₐₓ) | 0.7274 | 0.7299 | 99.66 | 88.77 | 111.89 | 0.936 | 19.09 |
| ln(AUCₗₐₛₜ) | 5.2960 | 5.1933 | 101.98 | 91.81 | 113.27 | 0.967 | 17.30 |
| ln(AUC_{inf}) | 5.3450 | 4.7971 | 111.42 | 99.05 | 125.34 | 0.930 | 15.64 |

^{a}Geometric Mean for Buprenorphine Sublingual Spray, 0.5 mg, Pre-treated with Cold Water (A), Pre treated with Hot Water (B), without Pre-treatment (C), Pre-treated with a Low pH Beverage (D), and Pre-treated with a High pH Beverage (E) based on Least Squares Mean of log-transformed parameter values
^{b}Ratio(%) = Geometric Mean (Test)/Geometric Mean (Ref)
^{c}90% Confidence Interval

TABLE 96
Statistical Analysis of the Log-transformed Systemic Exposure Parameters of Norbuprenorphine.

| Dependent | Geometric Mean | | Ratio (%) | 90% CI^{c} | | | ANOVA |
|---|---|---|---|---|---|---|---|
| Variable | A | C | (A/C) | Lower | Upper | Power | CV % |
| ln(Cₘₐₓ) | 0.0635 | 0.0623 | 102.04 | 92.70 | 112.32 | 0.9845 | 15.78 |
| ln(AUCₗₐₛₜ) | 1.8370 | 1.8826 | 97.58 | 82.44 | 115.50 | 0.7052 | 28.08 |

Pre-Treated with Hot Water vs. Without Pre-Treatment

| Dependent | Geometric Mean^{a} | | Ratio (%)^{b} | 90% CI^{c} | | | ANOVA |
|---|---|---|---|---|---|---|---|
| Variable | B | C | (B/C) | Lower | Upper | Power | CV % |
| ln(Cₘₐₓ) | 0.0655 | 0.0623 | 105.26 | 95.62 | 115.86 | 0.9845 | 15.78 |
| ln(AUCₗₐₛₜ) | 2.1698 | 1.8826 | 115.26 | 97.38 | 136.42 | 0.7052 | 28.08 |

Pre-Treated with Low pH Beverage vs. Without Pre-Treatment

| Dependent | Geometric Mean^{a} | | Ratio (%)^{b} | 90% CI^{c} | | | ANOVA |
|---|---|---|---|---|---|---|---|
| Variable | D | C | (D/C) | Lower | Upper | Power | CV % |
| ln(Cₘₐₓ) | 0.0616 | 0.0623 | 98.99 | 89.45 | 109.54 | 0.9754 | 15.78 |
| ln(AUCₗₐₛₜ) | 1.7457 | 1.8826 | 92.73 | 77.62 | 110.79 | 0.6644 | 28.08 |

Pre-Treated with High pH Beverage vs Without Pre-Treatment

| Dependent | Geometric Mean^{a} | | Ratio (%)^{b} | 90% CI^{c} | | | ANOVA |
|---|---|---|---|---|---|---|---|
| Variable | E | C | (E/C) | Lower | Upper | Power | CV % |
| ln(Cₘₐₓ) | 0.0584 | 0.0623 | 93.73 | 85.15 | 103.17 | 0.9845 | 15.78 |
| ln(AUCₗₐₛₜ) | 1.6497 | 1.8826 | 87.63 | 74.04 | 103.72 | 0.7052 | 28.08 |

Note:
Due to lack of data, AUC_{inf} could not be analyzed.
^{a}Geometric Mean for Buprenorphine Sublingual Spray, 0.5 mg, Pre-treated with Cold Water (A), Pre treated with Hot Water (B), without Pre-treatment (C), Pre-treated with a Low pH Beverage (D), and Pre-treated with a High pH Beverage (E) based on Least Squares Mean of log-transformed parameter values
^{b}Ratio(%) = Geometric Mean (Test)/Geometric Mean (Ref)
^{c}90% Confidence Interval

### Results and Conclusions

### Buprenorphine Temperature Effect

Pretreatment with cold water (Treatment A) or hot water (Treatment B) did not alter maximum and total buprenorphine exposure after single dose administration of Buprenorphine Sublingual Spray (0.5 mg); the 90% confidence intervals for Cmax, AUClast, and AUCinf were contained entirely within the 80%-125% interval for both conditions.

### Buprenorphine pH Effect

Pretreatment with a low pH beverage (Treatment D) reduced maximum buprenorphine exposure by approximately 10% and the lower bound of the 90% confidence interval for Cmax fell slightly below the 80%-125% interval (79.78%). Pretreatment with a low pH beverage did not alter total buprenorphine exposure after single dose administration of Buprenorphine Sublingual Spray (0.5 mg); the 90% confidence intervals for AUClast and AUCinf were contained entirely within the 80%-125% interval for both conditions.

Pretreatment with a high pH beverage (Treatment E) did not alter maximum and total buprenorphine exposure after single dose administration of Buprenorphine Sublingual Spray (0.5 mg); the 90% confidence intervals for Cmax, AUClast, and AUCinf were contained entirely within the 80%-125% interval.

### Norbuprenorphine Temperature Effect

Pretreatment with cold water (Treatment A) did not alter maximum and total norbuprenorphine exposure after single dose administration of Buprenorphine Sublingual Spray (0.5 mg); the 90% confidence intervals for Cmax and AUClast, were contained entirely within the 80%-125% interval for both conditions.

Pretreatment with hot water (Treatment B) did not alter maximum norbuprenorphine exposure after single dose administration of Buprenorphine Sublingual Spray (0.5 mg); the 90% confidence intervals for Cmax were contained entirely within the 80%-125% interval for both conditions. Pretreatment with hot water (Treatment B) increased total norbuprenorphine exposure by approximately 15% and the upper bound of the 90% confidence interval for AUClast fell above the 80%-125% interval (136.42%).

### Norbuprenorphine pH Effect

Pretreatment with a low pH beverage (Treatment D) or a high pH beverage (Treatment E) did not alter maximum norbuprenorphine exposure after single dose administration of Buprenorphine Sublingual Spray (0.5 mg); the 90% confidence intervals for Cmax were contained entirely within the 80%-125% interval for both conditions.

Pretreatment with a low pH beverage (Treatment D) or a high pH beverage (Treatment E) reduced total norbuprenorphine exposure by approximately 7% and 12%, respectively (0.5 mg) and the lower bounds of the 90% confidence intervals for AUClast fell below the 80%-125% interval (77.62% and 74.04%, respectively).

### Example 16. Pharmacokinetic Study of Buprenorphine Sublingual Sprays

The buprenorphine formulations described in Example 11, Table 77 of the instant specification were used. For formulation #29 and #30 was administered as a 0.5 mg dose and 1.0 mg dose.

Specifically, a single dose, open-label, two-period, two-treatment, ascending study to compare the bioavailability of ascending doses of Buprenorphine Sublingual Spray. Each dose of study treatment was separated by a washout period of 14 days. 20 subjects received 0.5 mg spray in period 1 and a 1.0 mg spray in period 2.

### Pharmacokinetic and Bioavailability Analysis

### Method

Each dose of buprenorphine sublingual spray (0.5 mg and 1.0 mg) was delivered as a single 100-μL spray developed by Insys Therapeutics, Inc. USA for investigational use only.

Each dose was administered following a 10-hour overnight fast. No food was allowed until 4 hours after dose administration. No water was consumed from 1 hour prior through 1 hour after dose. Meals were the same and scheduled at approximately the same times relative to dose for each study period. For each period, subjects were confined before dosing to ensure adherence to the 10-hour fast, and remained confined through the end of procedures for each period. In each study period, blood samples were collected for the quantitation of buprenorphine and norbuprenorphine at 0 (predose), at 5 minutes, 10 minutes, 15 minutes, 30 minutes, and 1, 2, 4, 8, 12, 24, 48, 72, 96, 120, and 144 hours post dose (16 time points).

Plasma samples were analyzed by WCT using validated LC-MS-MS procedures. The methods were validated for ranges of 0.0125 to 2.50 ng/mL for buprenorphine and 0.0200 to 4.00 ng/mL for norbuprenorphine, based on the analysis of 0.500 mL of human EDTA plasma. Data were stored in Watson Laboratory Information Management System™ (LIMS; Version 7.2.0.03, Thermo Fisher Scientific).

The following pharmacokinetic parameters were calculated: peak concentration in plasma (Cmax), time to peak concentration (Tmax), elimination rate constant (λz), terminal half-life (t½), area under the concentration-time curve from time-zero to the time of the last quantifiable concentration (AUClast), area under the plasma concentration time curve from time-zero extrapolated to infinity (AUC0-inf), the percent of AUCinf based on extrapolation (AUCextrap), last quantifiable plasma concentration (Clast), and time of the last quantifiable plasma concentration (Tlast).

TABLE 97
Buprenorphine Concentration-Time Data after Administration of Buprenorphine 0.5 mg
Sublingual Spray (Treatment A) and Buprenorphine 1.0 mg Sublingual Spray (Treatment B)

| | Treatment A: | | | | Treatment B: | | | |
|---|---|---|---|---|---|---|---|---|
| | | Buprenorphine | | | | Buprenorphine | | |
| Time | | Sublingual | | CV | | Sublingual | | CV |
| (h) | n | 0.5 mg Spray | | (%) | n | 1 .0 mg Spray | | (%) |
| 0.00 | 20 | 0.00 | 0.00 | NC | 17 | 0.00 | 0.00 | NC |
| 0.08 | 20 | 0.00 | 0.00 | NC | 17 | 0.0061 | 0.021 | 342.9 |
| 0.17 | 19 | 0.0562 | 0.0334 | 59.34 | 17 | 0.0722 | 0.072 | 100.1 |
| 0.25 | 20 | 0.130 | 0.0646 | 49.61 | 17 | 0.204 | 0.158 | 77.47 |
| 0.50 | 20 | 0.406 | 0.224 | 55.16 | 17 | 0.596 | 0.279 | 46.78 |
| 1.00 | 20 | 0.571 | 0.192 | 33.59 | 17 | 0.924 | 0.292 | 31.60 |
| 2.00 | 20 | 0.612 | 0.159 | 25.92 | 17 | 1.14 | 0.338 | 29.62 |
| 4.00 | 20 | 0.379 | 0.0943 | 24.90 | 17 | 0.725 | 0.172 | 23.66 |
| 8.00 | 20 | 0.113 | 0.0255 | 22.60 | 17 | 0.234 | 0.081 | 34.91 |
| 12.00 | 20 | 0.0614 | 0.0101 | 16.39 | 17 | 0.122 | 0.026 | 22.05 |
| 24.00 | 20 | 0.0284 | 0.0072 | 25.35 | 17 | 0.0548 | 0.017 | 31.33 |
| 48.00 | 20 | 0.00806 | 0.0105 | 130.1 | 17 | 0.0267 | 0.010 | 40.27 |

Note:
Plasma samples analyzed using a bioanalytical method with a validated range 0.0125 to 2.50 ng/mL; concentrations reported in ng/mL to 3 significant figures; concentrations below limit of quantification set to zero in the data summarization
NC = Not calculated

TABLE 98
Norbuprenorphine Concentration-Time Data after Administration of Buprenorphine 0.5 mg
Sublingual Spray (Treatment A) and Buprenorphine 1.0 mg Sublingual Spray (Treatment B)

| | Treatment A: | | | | Treatment B: | | | |
|---|---|---|---|---|---|---|---|---|
| | | Buprenorphine | | | | Buprenorphine | | |
| Time | | Sublingual | | CV | | Sublingual | | CV |
| (h) | n | 0.5 mg Spray | | (%) | n | 1.0 mg Spray | | (%) |
| 0.00 | 19 | 0.00 | 0.00 | NC | 16 | 0.00 | 0.00 | NC |
| 0.08 | 19 | 0.00 | 0.00 | NC | 16 | 0.00 | 0.00 | NC |
| 0.17 | 18 | 0.00 | 0.00 | NC | 16 | 0.00 | 0.00 | NC |
| 0.25 | 19 | 0.00 | 0.00 | NC | 16 | 0.00179 | 0.0071 | 400.0 |
| 0.50 | 19 | 0.0063 | 0.013 | 204.1 | 16 | 0.0177 | 0.0235 | 133.0 |
| 1.00 | 19 | 0.0249 | 0.019 | 79.73 | 16 | 0.0546 | 0.0363 | 66.48 |
| 2.00 | 19 | 0.0547 | 0.035 | 64.63 | 16 | 0.0915 | 0.0561 | 61.27 |
| 4.00 | 19 | 0.0473 | 0.020 | 43.35 | 16 | 0.102 | 0.0556 | 54.61 |
| 8.00 | 19 | 0.0408 | 0.015 | 38.56 | 16 | 0.0836 | 0.0324 | 38.71 |
| 12.00 | 19 | 0.0388 | 0.016 | 43.56 | 16 | 0.0834 | 0.0325 | 39.05 |
| 24.00 | 19 | 0.0300 | 0.022 | 75.07 | 16 | 0.0812 | 0.0408 | 50.27 |
| 48.00 | 19 | 0.0077 | 0.014 | 183.9 | 16 | 0.0469 | 0.0342 | 72.90 |

Note:
Plasma samples analyzed using a bioanalytical method with a validated range 0.0200 to 4.00 ng/mL; concentrations reported in ng/mL to 3 significant figures; concentrations below limit of quantification set to zero in the data summarization
NC = not calculated

TABLE 99
Pharmacokinetic Parameters of Buprenorphine

| | Treatment A: Buprenorphine | | | | Treatment B: | | | |
|---|---|---|---|---|---|---|---|---|
| | Sublingual | | | | Buprenorphine Sublingual | | | |
| | 0.5 mg Spray | | | | 1.0 mg Spray | | | |
| Parameter | | Mean | SD | CV % | | Mean | SD | CV % |
| Tₘₐₓ (h) | 20 | 1.43 | 0.62 | 43.15 | 17 | 1.71 | 0.47 | 27.61 |
| Cₘₐₓ (ng/mL) | 20 | 0.660 | 0.200 | 30.35 | 17 | 1.17 | 0.329 | 28.06 |
| Cₘₐₓ/Dose | 20 | 1.32 | 0.401 | 30.35 | 17 | 1.17 | 0.329 | 28.06 |
| (ng/mL/mg) | | | | | | | | |
| AUCₗₐₛₜ | 20 | 4.043 | 0.8170 | 20.21 | 17 | 8.053 | 1.653 | 20.52 |
| (h*ng/mL) | | | | | | | | |
| AUCₗₐₛₜ/Dose | 20 | 8.086 | 1.634 | 20.21 | 17 | 8.053 | 1.653 | 20.52 |
| (h*ng/mL/mg) | | | | | | | | |
| AUC_{inf} | 20 | 4.521 | 1.233 | 27.26 | 17 | 8.715 | 1.861 | 21.35 |
| (h*ng/mL) | | | | | | | | |
| AUC_{inf}/Dose | 20 | 9.042 | 2.465 | 27.26 | 17 | 8.715 | 1.861 | 21.35 |
| (h*ng/mL/mg) | | | | | | | | |
| AUC_{Extrap} (%) | 20 | 9.55 | 6.46 | 67.68 | 17 | 7.41 | 2.81 | 37.87 |
| λ_{z} (h⁻¹) | 20 | 0.0659 | 0.0325 | 49.32 | 17 | 0.0484 | 0.0218 | 45.06 |
| T_{1/2} (h) | 20 | 14.07 | 10.06 | 71.46 | 17 | 16.00 | 4.34 | 27.12 |
| Tₗₐₛₜ (h) | 20 | 34.81 | 12.26 | 35.21 | 17 | 46.59 | 5.82 | 12.50 |
| Cₗₐₛₜ (ng/mL) | 20 | 0.0222 | 0.00793 | 35.78 | 17 | 0.0277 | 0.00879 | 31.77 |

TABLE 100
Pharmacokinetic Parameters of Buprenorphine

| | Treatment A: | | | | Treatment B: | | | |
|---|---|---|---|---|---|---|---|---|
| | Buprenorphine Sublingual | | | | Buprenorphine Sublingual | | | |
| | 0.5 mg Spray | | | | 1.0 mg Spray | | | |
| Parameter | | Mean | SD | CV % | | Mean | SD | CV % |
| Tₘₐₓ (h) | 19 | 6.48 | 10.61 | 163.75 | 16 | 6.08 | 5.73 | 94.22 |
| Cₘₐₓ (ng/mL) | 19 | 0.0599 | 0.0326 | 54.45 | 16 | 0.115 | 0.0553 | 48.20 |
| Cₘₐₓ/Dose | 19 | 0.120 | 0.0652 | 54.45 | 16 | 0.115 | 0.0553 | 48.20 |
| (ng/mL/mg) | | | | | | | | |
| AUCₗₐₛₜ | 19 | 1.113 | 0.7857 | 70.56 | 16 | 3.405 | 1.736 | 50.98 |
| (h*ng/mL) | | | | | | | | |
| AUCₗₐₛₜ/Dose | 19 | 2.227 | 1.571 | 70.56 | 16 | 3.405 | 1.736 | 50.98 |
| (h*ng/mL/mg) | | | | | | | | |
| AUC_{inf} | 12 | 4.374 | 3.709 | 84.81 | 12 | 7.877 | 4.725 | 59.99 |
| (h*ng/mL) | | | | | | | | |
| AUC_{inf}/Dose | 12 | 8.748 | 7.418 | 84.81 | 12 | 7.877 | 4.725 | 59.99 |
| (h*ng/mL/mg) | | | | | | | | |
| AUC_{Extrap} (%) | 12 | 61.21 | 25.09 | 40.99 | 12 | 53.03 | 19.94 | 37.61 |
| λ_{z} (h⁻¹) | 12 | 0.0242 | 0.0176 | 72.65 | 12 | 0.0193 | 0.0146 | 75.74 |
| T_{1/2} (h) | 12 | 61.29 | 63.56 | 103.70 | 12 | 56.65 | 36.73 | 64.84 |
| Tₗₐₛₜ (h) | 19 | 27.01 | 14.14 | 52.34 | 16 | 42.01 | 10.73 | 25.54 |
| Cₗₐₛₜ (ng/mL) | 19 | 0.0316 | 0.0110 | 34.83 | 16 | 0.0561 | 0.0230 | 41.04 |

TABLE 101
Statistical Analysis of the Log-Transformed Systemic Exposure Parameters of
Buprenorphine

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ/Dose) | 1.2638 | 1.1584 | 109.10 | 98.43 | 120.92 | 0.9710 | 17.30 |
| ln(AUCₗₐₛₜ/Dose | 7.9326 | 7.8709 | 100.78 | 91.03 | 111.58 | 0.9731 | 17.12 |
| ln(AUC_{inf}/Dose) | 8.7938 | 8.4938 | 103.53 | 92.92 | 115.35 | 0.9592 | 18.20 |

^{a}Geometric Mean for 0.5 mg Buprenorphine (Test) and 1.0 mg Buprenorphine (Ref) based on Least Squares Mean of log-transformed parameter values
^{b}Ratio(%) = Geometric Mean (Test)/Geometric Mean (Ref)
^{c}90% Confidence Interval

TABLE 102
Statistical Analysis of the Log-Transformed Systemic Exposure Parameters of
Norbuprenorphine

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ/Dose) | 0.107 | 0.109 | 98.68 | 87.20 | 111.6 | 0.9104 | 20.1 |
| ln(AUCₗₐₛₜ/Dose | 1.691 | 2.872 | 58.88 | 46.84 | 74.02 | 0.4849 | 38.2 |
| ln(AUC_{inf}/Dose | 6.329 | 5.162 | 122.62 | 46.82 | 321.1 | 0.1176 | 81.5 |

^{a}Geometric Mean for 0.5 mg Buprenorphine (Test) and 1.0 mg Buprenorphine (Ref) based on Least Squares Mean of log-transformed parameter values
^{b}c Mean (Test)/Geometric Mean (Ref)
^{c}e Interval

Results

The first quantifiable buprenorphine concentrations were observed at the 0.08-hour sample time for the 1.0 mg buprenorphine sublingual spray (Treatment B) and at 0.17 hour sample time for the 0.5 mg buprenorphine sublingual spray (Treatment A). The highest mean plasma concentrations were 0.612±0.159 ng/mL for the 0.5 mg buprenorphine sublingual spray (Treatment A) and 1.14±0.338 ng/mL for the 1.0 mg buprenorphine sublingual spray (Treatment B), both at 2.00 h. Quantifiable buprenorphine concentrations were observed throughout the 48.00 hour sampling interval for most subjects.

The first quantifiable norbuprenorphine concentrations were observed at the 0.25-hour sample time for the 1.0 mg buprenorphine sublingual spray (Treatment B) and at 0.50-hour sample time for the 0.5 mg buprenorphine sublingual spray (Treatment A). The highest mean plasma concentrations were 0.0547±0.0353 ng/mL at 2.00 h for the 0.5 mg buprenorphine sublingual spray (Treatment A) and 0.102±0.0556 ng/mL at 4.00 h for the 1.0 mg buprenorphine sublingual spray (Treatment B) h. Quantifiable norbuprenorphine concentrations were observed throughout the 48.00 hour sampling interval for some subjects.

What is claimed is:

1. A liquid formulation comprising from about 0.05% to about 10% w/w buprenorphine, a pharmaceutically acceptable salt thereof, or a derivative thereof, water as a solvent, and a mixture of an alcohol and a glycol as a cosolvent.

2. The liquid formulation of claim 1 further comprising naloxone, a pharmaceutically acceptable salt thereof, or a derivative thereof.

3. The liquid formulation of claim 1, wherein the formulation is a liquid spray formulation.

4. The liquid formulation of claim 1, further comprising an antioxidant.

5. The liquid formulation of claim 4, wherein the antioxidant is selected from the group consisting of butylated hydroxyanisole (BHA), butylated hydroxytoluene (BHT), methionine, sodium ascorbate, sodium thiosulfate, thioglycerol, cysteine hydrochloride monohydrate and a mixture thereof.

6. A sublingual spray formulation comprising:
buprenorphine, a pharmaceutically acceptable salt thereof or a derivative thereof in an amount from about 0.07% to about 1.3% w/w;
water as a solvent in an amount from about 38% to about 40% w/w;
a cosolvent consisting of a mixture of ethanol in an amount of about 55% w/w and propylene glycol in an amount of about 5% w/w; and
an antioxidant in an amount from about 0.0001% to about 0.5% w/w, wherein the % w/w is of the total formulation.

7. The formulation of claim 6 further comprising from about 0.005% to about 0.5% w/w menthol.

8. The formulation of claim 7, wherein:
the antioxidant consists of a mixture of butylated hydroxyanisole (BHA) in an amount of about 0.01% w/w and butylated hydroxytoluene (BHT) in an amount of about 0.005% w/w; and
menthol is at an amount of about 0.05% w/w.

9. The liquid formulation of claim 1 wherein the formulation is a sublingual spray formulation and is capable of producing a droplet size distribution wherein greater than 98% of the composition particles are greater than 10 microns in diameter during administration.

10. The liquid formulation of claim 1 wherein the formulation is a sublingual spray formulation and is capable of producing a droplet size distribution wherein:
the mean Dv(10) is from about 10 to about 40 microns during administration;
the mean Dv(50) is from about 30 to about 80 microns during administration; and
the mean Dv(90) is from about 80 to about 200 microns during administration.

11. The liquid formulation of claim 1 wherein the formulation is a sublingual spray formulation and is capable of producing a spray plume that has an ovality ratio of from about 1.1 to 2.4.

12. The liquid formulation of claim 1 wherein the formulation is capable of producing a spray plume width that is from about 25 to about 45 millimeters during administration and a spray plume angle that is from about 30 to about 55 degrees during administration.

13. The liquid formulation of claim 1 that is capable of producing a D(4,3) of 50 to 95 microns.

14. The liquid formulation of claim 1 wherein the formulation is a sublingual spray formulation that is capable of producing a droplet size distribution wherein the Cₘₐₓ (ng/mL) of buprenorphine is from 0.125±0.0203 to 1.57±0.453.

15. The liquid formulation of claim 1 wherein the formulation is a sublingual spray formulation that is capable of producing a droplet size distribution wherein the Tₘₐₓ of buprenorphine is from about 0.5 to about 2.0 hours following administration.

16. The liquid formulation of claim 1 wherein the formulation is a sublingual spray formulation that is capable of producing a droplet size distribution wherein the AUCinf of buprenorphine (h*ng/mL) is from 0.6387±0.1844 to 11.36±3.153.

17. The liquid formulation of claim 2 wherein the formulation is a sublingual spray formulation that is capable of producing a droplet size distribution wherein the Cmax (ng/mL) of buprenorphine is from 2470±850 to 5670±1590 and the Cmax (ng/mL) of naloxone is from 4.26±2.52 to 12.0±5.38.

18. The liquid formulation of claim 2 wherein the formulation is a sublingual spray formulation that is capable of producing a droplet size distribution wherein the Tmax of buprenorphine is from 1.63±0.5 to 1.68±0.73 hours and the Tmax of naloxone is from about 1.17 to about 1.40 hours following administration.

19. The liquid formulation of claim 2 wherein the formulation is a sublingual spray formulation that is capable of producing a droplet size distribution wherein the AUCinf (h*ng/mL) of buprenorphine is from 19320±6190 to 48970±13810 and the AUCinf (h*ng/mL) of naloxone is from 11.87±3.903 to 36.22±10.45.

20. A method of treating pain comprising administering the liquid formulation of claim 1 to a patient in need thereof.

21. A method of treating opioid dependence comprising administering the liquid formulation of claim 2 to a patient in need thereof.
